(19) United States
(12) Patent Application Publication
(10) Pub. No.: US 2006/0292135 A1
(43) Pub. Date: Dec. 28, 2006

(54) Use of bacterial phage-associated lysing proteins for preventing and treating bacterial infections in humans, animals and fowl

(76) Inventors: Lawrence Loomis, Columbia, MD (US); Vincent Fischetti, West Hempstead, NY (US)

Correspondence Address: Jonathan E. Grant, 2107 HOUNDS RUN PLACE, Silver Springs, MD, 20906, US

(21) Appl. No.: 11/186,018

(22) Filed: Jul. 18, 2005

Related U.S. Application Data
(63) Continuation of application No. PCT/US04/01077, filed on Jan. 16, 2004.
(63) Continuation-in-part of application No. 09/671,881, filed on Sep. 28, 2000, now Pat. No. 6,428,784.
Application No. 09/671,881 is a continuation-in-part of application No. 09/497,495, filed on Apr. 18, 2000, now Pat. No. 6,238,661.
Application No. 09/497,495 is a continuation of application No. 09/395,636, filed on Sep. 14, 1999, now Pat. No. 6,056,954.
Application No. 09/395,636 is a continuation-in-part of application No. 08/962,523, filed on Oct. 31, 1997, now Pat. No. 5,997,862.
(60) Provisional application No. 60/440,352, filed on Jan. 16, 2003.

(51) Int. Cl. A61K 38/43; A61K 9/00

(52) U.S. Cl. 424/94.1; 435/183; 424/400

(57) ABSTRACT

A composition and method for treating bacterial infections by the use of an effective amount of at least one lytic specific for the bacteria causing specific. The lytic enzyme is genetically coded for by a bacteriophage which may be specific for said bacteria. The enzyme may be at least one lytic protein or peptides in a natural or modified form.

This application claims priority to U.S. provisional application No. 60/440,352 filed on Jan. 16, 2003, the entirety of which is hereby incorporated by reference.

### BACKGROUND

1. Field of the Disclosure

The disclosure relates to methods and compositions for treating bacterial infections with bacteria-associated phage proteins, enzymes or peptides, and/or peptide fragments thereof. More specifically, the disclosure pertains to phage lytic and/or holin proteins, or peptides and peptide fragments thereof, blended with a carrier for the treatment and prophylaxis of bacterial infections.

2. Description of the Prior Art

A major problem in medicine has been the development of drug resistant bacteria as more antibiotics are used for a wide variety of illnesses and other conditions. The over utilization of antibiotics has increased the number of bacteria showing resistance. Furthermore, broadly reactive antibiotics can effect normal flora and can cause antibiotic resistance in these organisms because of the frequency of drug use. The number of people becoming hyper allogenic to antibiotics appears to be increasing because of antibiotic overutilization. Accordingly, there is a commercial need for new antibiotics (or bacterial killing substances), especially those that operate in new modalities or provide new means to kill pathogenic bacteria.

Attempts have been made to treat bacterial diseases through the use of bacteriophages.

U.S. Pat. No. 5,688,501 (Merril, et al.) discloses a method for treating an infectious disease caused by bacteria in an animal with lytic or non-lytic bacteriophages that are specific for particular bacteria.

U.S. Pat. No. 4,957,686 (Norris) discloses a procedure of improved dental hygiene which comprises introducing into the mouth bacteriophages parasitic to bacteria which possess the property of readily adhering to the salivary pellicle.

However, the direct introduction of bacteriophages to prevent or fight diseases has certain drawbacks. Specifically, both the bacteria and the phage have to be in the correct and synchronized growth cycles for the phage to attach to the bacteria. Additionally, there must be the right number of phages to attach to the bacteria; if there are too many or too few phages, there will be no attachment and no production of the lysing enzyme. The phage must also be sufficiently active. Phages are inhibited by bacterial debris from the invaded organism which block the phage's attachment site to its receptor. Further complicating the direct use of a bacteriophage to treat bacterial infections is the possibility of immunological reactions, rendering the phage non-functional. Most importantly, the targeted bacteria mutates its surface receptors for the bacteriophage, rendering the bacteria non-infective.

Consequently, others have explored the use of safer and more effective means to treat and prevent bacterial infections. In particular, the use of phage associated lytic enzymes has been explored.

Bacteriophage lysins are a class of bacteriolytic agents recently proposed for eradicating the nasopharyngeal carriage of pathogenic streptococci. (Loeffler, J. M., Nelson, D. & Fischetti, V. A. Rapid killing of Streptococcus pneumoniae with a bacteriophage cell wall hydrolase. Science 294, 2170-2 (2001); Nelson, D., Loomis, L. & Fischetti, V. A. Prevention and elimination of upper respiratory colonization of mice by group A streptococci by using a bacteriophage lytic enzyme. Proc Natl Acad Sci U S A 98, 4107-12 (2001)). Lysins are part of the lytic mechanism used by double stranded DNA (dsDNA) phage to coordinate host lysis with completion of viral assembly. Wang, I. N., Smith, D. L. & Young, R. Holins: the protein clocks of bacteriophage infections. Annu Rev Microbiol 54, 799-825 (2000). Late in infection, lysin translocates into the cell wall matrix where it rapidly hydrolyzes covalent bonds essential for peptidoglycan integrity, causing bacterial lysis and concomitant progeny phage release. Lysin family members exhibit a modular design in which a usually well conserved catalytic domain is fused to a more divergent specificity or binding domain. See, Lopez, R., Garcia, E., Garcia, P. & Garcia, J. L. The pneumococcal cell wall degrading enzymes: a modular design to create new lysins? Microb Drug Resist 3, 199-211 (1997); Loessner, M. J., Kramer, K., Ebel, F. & Scherer, S. C-terminal domains of Listeria monocytogenes bacteriophage murein hydrolases determine specific recognition and high-affinity binding to bacterial cell wall carbohydrates. Mol Microbiol 44, 335-49 (2002). In many cases a fragment having catalytic activity can be determined and is preferably linked to a binding site region. The linkage optionally may be made through a third joining piece. High affinity binding is directed towards species- or strain-specific cell wall carbohydrate ligands that are often essential for bacterial viability, thus implying that intrinsic lysin resistance will be rare or impossible.

U.S. Pat. No. 5,604,109 (Fischetti et al.) relates to the rapid detection of Group A streptococci in clinical specimens, through the enzymatic digestion of the bacterial cell wall by a semi-purified Group C streptococcal phage associated lysin enzyme. Embodiments of the disclosure are based upon the discovery that phage associated lytic enzymes specific for bacteria infected with a specific phage can effectively and efficiently break down the cell wall of the bacterium in question. At the same time, in most if not all cases, the semi purified enzyme is lacking mammalian cell receptors and therefore tends to be less destructive to mammalian proteins and tissues when present during the digestion of the bacterial cell wall.

U.S. Pat. No. 5,985,271 (Fischetti & Loomis), U.S. Pat. No. 5,997,862 (Fischetti & Loomis), and U.S. Pat. No. 6,017,528 (Fischetti & Loomis) disclose the compositions and their use in an oral delivery mode, such as a candy, chewing gum, lozenge, troche, tablet, a powder, an aerosol, a liquid or a liquid spray that contains a lysin enzyme produced by group C streptococcal bacteria infected with a C1 bacteriophage for the prophylactic and therapeutic treatment of Streptococcal A throat infections, commonly known as strep throat. This lysin enzyme is described in U.S. Pat. No. 5,604,109.

U.S. Pat. No. 6,056,954 (Fischetti & Loomis) discloses a method and composition for the prophylactic and/or therapeutic treatment of bacterial infections that comprises the administration of an effective amount of at least one lytic enzyme produced by a bacteria infected with a bacteriophage specific for the bacteria The lytic enzyme preferably comprises a carrier suitable for delivering the lytic enzyme to the site of the infection. This method and treatment may be used for treating and eliminating bacterial infestations anywhere, including upper respiratory infections, topical and systemic infections, vaginal infections, eye infections, ear infections, infections requiring parenteral treatment, as well as for the elimination of bacteria on any surface.

U.S. Pat. No. 6,056,955 (Fischetti and Loomis) discloses a method and composition for the topical treatment of streptococcal infections by the use of a lysin enzyme blended with a carrier suitable for topical application to dermal tissues. The method for the treatment of streptococcal infections describes the administration of a composition comprising an effective amount of a therapeutic agent, where the therapeutic agent is a lysin enzyme produced by group C streptococcal bacteria infected with a Cl bacteriophage. The therapeutic agent can be in a pharmaceutically acceptable carrier.

U.S. Pat. No. 6,248,324 (Fischetti and Loomis) discloses a method for treatment of bacterial infections of the digestive tract comprising the administration of a lytic enzyme specific for the infecting bacteria. The lytic enzyme is preferably in a carrier for delivering said lytic enzyme. The bacteria species to be treated is selected from the group consisting of Listeria, Salmonella, E. coli, Campylobacter, and combinations thereof. The carrier for delivering at least one lytic enzyme to the digestive tract is selected from the group consisting of suppositories, enemas, syrups, or enteric coated pills.

U.S. Pat. No. 6,254,866 (Fischetti and Loomis ) discloses a method for treating bacterial infections of the digestive tract comprising the administration of a lytic enzyme specific for the infecting bacteria. There is preferably a carrier for delivering the lytic enzyme to the site of the infection in the digestive tract. The bacteria to be treated is selected from the group consisting of Listeria, Salmonella, E. coli, Campylobacter, and combinations thereof. The carrier is selected from the group consisting of suppositories, enemas, syrups, or enteric coated pills.

U.S. Pat. No. 6,264,945 (Fischetti and Loomis) discloses a method and composition for the treatment of bacterial infections by the parenteral introduction of at least one phage associated lytic enzyme specific for the invasive bacteria and an appropriate carrier for delivering the lytic enzyme into a patient. The injection can be done intramuscularly, subcutaneously, or intravenously.

U.S. Pat. No. 6,238,661 (Fischetti and Loomis) discloses compositions and methods for the prophylactic and therapeutic treatment of bacterial infections which comprise administering to an individual an effective amount of a composition comprising an effective amount of lytic enzyme and a carrier for delivering the lytic enzyme. This method and composition can be used for the treatment of upper respiratory infections, skin infections, wounds, burns, vaginal infections, eye infections, intestinal disorders and dental problems.

Embodiments of the disclosure are based upon the discovery that phage associated lytic enzymes specific for bacteria infected with a specific phage can effectively and efficiently break down the cell wall of the bacterium in question. At the same time, in most if not all cases, the semi or fully purified enzyme is lacking in mammalian cell receptors and therefore tends to be less destructive to mammalian proteins and tissues when present during the digestion of the bacterial cell wall.

The same general technique used to produce and purify a lysin enzyme shown in U.S. Pat. No. 5,604,109 may be used to manufacture other lytic enzymes produced by bacteria infected with a bacteriophage specific for that bacteria. Depending on the bacteria, there may be variations in the growth media and conditions.

The use of phage associated lytic enzymes produced by the infection of a bacteria with a bacteria specific phage has numerous advantages for the treatment of diseases. The lytic enzymes are targeted against a specific bacteria and these do not interfere with normal flora. Also, lytic enzymes primarily attack cell wall structures, which are not affected by plasmid variation. The actions of the lytic enzymes are fast and do not depend on bacterial growth. Additionally, lytic enzymes can be directed to the mucosal lining, where, in residence, they can kill colonizing bacteria.

### SUMMARY OF THE DISCLOSURE

The present disclosure further discloses that bacteriophage associated lytic proteins and holin proteins may be used to treat and prevent bacterial diseases. The phage associated lytic and holin proteins include their isozymes, analogs, and variants thereof in a natural or modified form either alone or in combination with complementary agents. The disclosure also features compositions that are site-specific for the mucosal membranes and pharmaceutically acceptable carriers for the treatment and amelioration for infections of the mucus membrane.

Accordingly, the present disclosure provides a pharmaceutical composition containing at least one bacteria-associated phage protein and peptides and peptide fragments thereof, isolated from one or more bacteria species, wherein the phage proteins and peptide fragments thereof include phage lytic and/or holin proteins. In one embodiment of the disclosure, the lytic and/or holin proteins, including their isozymes, analogs, or variants, are used in an altered form. In another embodiment of the disclosure, the lytic and/or holin proteins, including their isozymes, analogs, or variants, are used in a modified form or a combination of natural and modified forms. Tie altered forms of lytic and lion proteins are -made synthetically by chemical synthesis and/or DNA recombinant techniques.

The disclosure features compositions containing at least one natural lytic protein, including isozymes, analogs, or variants thereof, isolated from the same or:<different bacteria, with optional additions of a complementary agent.

According to one embodiment of the disclosure, the pharmaceutical composition includes one or more altered lytic protein(s), including isozymes, analogs, or variants thereof, produced by chemical synthesis or DNA recombinant techniques. In particular, altered, lytic protein is produced by chimerization, shuffling, or both. Preferably, the pharmaceutical composition contains combination(s) of one or more natural lytic protein and one or more chimeric or shuffled lytic protein(s).

According to another embodiment of the disclosure,-;the pharmaceutical composition contains a peptide or a peptide fragment of at least one lytic protein derived from the same or different bacterial species, with an optional addition of one or more complementary agents, and a pharmaceutically acceptable carrier.

Also within the scope of the disclosure are compositions containing nucleic acid molecules that either alone or in combination with other nucleic acid molecules are capable of expressing an effective amount of lytic and/or holin proteins or a peptide fragment of the lytic and/or holin proteins in vivo. Also within the scope of this disclosure are cell cultures containing these nucleic acid molecules polynucleotides and vectors carrying and expressing these molecules in vitro or in vivo.

According to another embodiment of the disclosure, the pharmaceutical composition contains a complementary agent, including one or more conventional antibiotics.

According to another aspect of the disclosure, the pharmaceutical composition contains antibodies directed against a phage protein or peptide fragment of the disclosure.

In yet another embodiment, the disclosure provides for the prevention, amelioration, or treatment of a variety of illnesses caused by Gram negative and/or Gram positive bacteria.

The bacteriophage associated proteins of this disclosure may be administered to subjects via several means of application. Means of application include suitable carriers that assist in delivery of the composition to the site of the infection and subsequent adsorption of the composition. The compositions containing lytic and/or holin proteins or peptides and peptide fragments thereof are incorporated into pharmaceutically acceptable carriers and are placed into appropriate means of application and delivery. Preferable application means include suppositories, enemas, liquid means (for example, syrups, mouthwash, and eye drops in aqueous or nonaqueous form), solid means (for example, food stuff, confectionary, and toothpaste), bandages, tampons, topical creams, and inhalers, among others.

According to one embodiment of the disclosure, one or more phage proteins, or peptides and peptide fragments thereof, are placed in an inhaler to treat or prevent the spread of diseases localized in the mucus lining of the oral cavity and lungs. In a preferred embodiment, specific lytic proteins for tuberculosis are placed in a carrier and used to prevent or treat tuberculosis. In another embodiment of the disclosure, phage proteins are administered in the form of candy, chewing gum, lozenge, troche, tablet, a powder, aerosol, liquid spray, or toothpaste for the prevention or treatment of bacterial infections associated with upper respiratory tract illnesses.

According to another embodiment of disclosure, eye drops containing lytic phage proteins of Hemophilus, Pseudomonas, Staphylococcus and other bacteria are used to directly prevent and/or treat eye infections.

In yet another embodiment of the disclosure, specific lytic proteins are used in the prevention and/or treatment of bacterial infections associated with topical or dermatological infections, administered in the form of a topical ointment or cream. The disclosure also provides a composition and method to treat or prevent infections of burns and wounds by using one or more phage lytic proteins, including preferably phage lytic proteins associated with Staphylococcus or Pseudomonas, incorporated into bandages to prevent or treat infections of burns and wounds. According to another embodiment, lytic proteins, including those proteins or peptides and peptide fragments specific for group B Streptococcus, are incorporated into tampons to prevent infection of the neonate during birth without distributing normal vaginal flora so that women would not be overcome by yeast infection as a result of antibiotic therapy.

According to yet another embodiment of the disclosure, the pharmaceutical composition(s) contain phage polypeptides, peptide fragments, nucleic acid molecules encoding phage protein or protein peptides fragments, antibody and antibody fragments, having biological activity either alone or with combination of other molecules polypeptides, peptides. In particular, the phage polypeptides are selected from the group consisting of: a natural phage polypeptide, a naturally occurring allelic variant of said polypeptide, a modified polypeptide, and a polypeptide which is encoded by a nucleic acid molecule comprising a nucleotide sequence which is at least 65% identical to a nucleic acid encoding the said natural peptide. Additionally, the polypeptide of the disclosure is attached to heterologous amino acid sequences.

According, to another embodiment of the disclosure, phage peptides and peptide fragments thereof are antibodies that selectively bind to phage polypeptides.

Embodiments of the disclosure also feature nucleic acid molecules as phage peptides and peptide fragments thereof. The nucleic acid molecules of the disclosure are preferably attached to regulatory sequences and signal sequences, wherein the sequences: affect site specificity and trans-membrane movements of the nucleic acid molecules. The signal sequences affect transportation of the nucleic acid molecules to the mucous membranes.

According to another aspect of the disclosure, a method for detecting the presence of a phage protein or peptides and peptide fragments thereof of the disclosure in a sample comprises: contacting the sample with a compound which selectively binds to the phage: protein or peptides and peptide fragments thereof and determining whether the compound binds to the phage protein or peptides and peptide fragments thereof in said sample. In a preferred embodiment the compound is an antibody.

According to another embodiment of the disclosure, the disclosure discloses a drug screening method for identifying a compound which binds to a polypeptide of the disclosure comprising the steps of: contacting a polypeptide, or a cell expressing a polypeptide of the disclosure with a test compound; and determining whether the polypeptide binds to the test compound. The drug screening method also includes methods for modulating the activity of a polypeptide of the disclosure, as disclosed and described herein, comprising contacting a polypeptide or a cell expressing a polypeptide of the disclosure with a compound which binds to the polypeptide in a sufficient concentration to modulate the activity of the polypeptide.

In yet another embodiment of the disclosure, holin proteins are used in conjunction with phage associated lytic enzymes to prophylactically and therapeutically treat bacterial diseases. In another embodiment of the disclosure, holin proteins alone are used to prophylactically and therapeutically treat bacterial infections. The holin proteins may be shuffled holin proteins or chimeric holin proteins, in either combination with or independent of the lytic enzymes.

In yet another embodiment of the disclosure, a chimeric and/or shuffled lytic enzyme is administered parenterally, wherein the phage associated lytic enzyme is administered intramuscularly, intrathecally, subdermally, subcutaneously, or intravenously to treat infections.

It is another object of the disclosure to apply a phage associated shuffled and/or chimeric lytic enzyme intravenously, to treat septicemia and general infections.

In yet another embodiment, chimeric lytic enzymes, shuffled lytic enzymes, “unaltered” versions of the lytic enzymes, holin proteins, and combinations thereof are used to prophylactically and therapeutically treat exposure to bacteria. In another embodiment, chimeric lytic enzymes, shuffled lytic enzymes, “unaltered” versions of the lytic enzymes, holin proteins, and combinations thereof are used to detect and identify specific bacteria. In one embodiment, the phage associated lytic enzyme specific for specific bacteria may be used to identify specific bacteria in its vegetative state.

Some sequences that have been isolated from the phage are shown in this disclosure, however other lytic enzymes from other bacteriophage specific for the same bacteria may be used in place of die sequenced lytic enzyme. In one embodiment, the DNA encoding the lytic enzyme or holin protein, including their isozymes, analogs, or variants, has been genetically altered. In another embodiment, the lytic enzyme or holin protein, including their isozymes, analogs, or variants, has been chemically altered. In yet another embodiment, the lytic enzyme or holin protein, including their isozymes, analogs, or variants, are used in a combination of natural and modified (genetically or chemically altered) forms. The altered forms of lytic enzymes and holin proteins are made synthetically by chemical synthesis and/or DNA recombinant techniques. The enzymes are made synthetically by chimerization and/or shuffling.

It should be understood that bacteriophage lytic enzymes have specifically cleave bonds that are present in the peptidoglycan of bacterial cells. Since the bacterial cell wall peptidoglycan is highly conserved among all bacteria, there are only a few bonds to be cleaved to disrupt the cell wall. Enzymes that cleave these bonds are muramidases, glucosaminidases, endopeptidases, or N-acetyl-muramoyl-L-alanine amidases (hereinafter referred to as amidases). The majority of reported phage enzymes are either muramidases or amidases, and there have been no reports of bacteriophage glucosaminidases. Fischetti et al (1974) reported that the C1 streptococcal phage lysin enzyme was an amidase. Garcia et al (1987, 1990) reported that the Cp1 lysin from a S. pneumoniae from a Cp-1 phage was a lysozyme. Caldentey and Bamford (1992) reported that a lytic enzyme from the phi 6 Pseudomonas phage was an endopeptidase, splitting the peptide bridge formed by melo-diaminopimilic acid and D-alanine. The E. coli T1 and T6 phage lytic enzymes are amidases as is the lytic enzyme from Listeria phage (ply) (Loessner et al, 1996). There are also other enzymes which cleave the cell wall.

Another embodiment of the present invention also provides for chimeric proteins or peptides fragments which include fusion proteins for the aforesaid uses.

A definition of terms used and their applicability to the disclosure are provided as follows:

Phage enzymes or proteins, as disclosed herein, include phage polypeptides, peptide fragments, nucleic acid molecules encoding phage protein or protein peptide fragments, antibody and antibody fragments, having biological activity either alone or with combination of other molecules. When reference is made to lytic enzymes, the enzyme may include any form of the peptide that allows for the destruction of the cell wall under the specified conditions.

Nucleic acid molecules, as disclosed herein, include genes, gene fragments polynucleotides, oligonucleotides, DNA, RNA, DNA-RNA hybrids, EST, SNIPs, genomic DNA, cDNA, mRNA, antisense RNA, ribozyme vectors containing nucleic acid molecules, regulatory sequences, and signal sequences. Nucleic acid molecules of this disclosure include any nucleic acid-based molecule that either alone or in combination with other molecules produces an oligonucleotide molecule capable or incapable of translation into a peptide.

In this context of the embodiments, the term “lytic enzyme genetically coded for by a bacteriophage” means a polypeptide having at least some lytic activity against the host bacteria. The polypeptide has a sequence that encompasses a native sequence of a lytic enzyme and variants thereof. The polypeptide may be isolated from a variety of sources, such as from phage, or prepared by recombinant or synthetic methods, such as those by Garcia et al. Every polypeptide (lytic enzyme) has two domains. One domain is a cell wall binding portion at the carboxyl terminal side and the other domain is an amidase activity that acts upon amide bonds in the peptidoglycan at the amino terminal side. Generally speaking, a lytic enzyme according to the disclosure is between 25,000 and 35,000 daltons in molecular weight and comprises a single polypeptide chain; however, this can vary depending on the enzyme chain. The molecular weight is determined by assay using sodium dodecyl sulfate gel electrophoresis and comparison with molecular weight markers.

The term “isolated” means at least partially purified from a starting material. The term “purified” means that the biological material has been measurably increased in concentration by any purification process, including by not limited to, column chromatography, HPLC, precipitation, electrophoresis, etc., thereby partially, substantially or completely removing impurities such as precursors or other chemicals involved in preparing the material. Hence, material that is homogenous or substantially homogenous (e.g., yields a single protein bond in a separation procedure such as electrophoresis or chromatography) is included within the meanings of isolated and purified. The amount of purification necessary will depend upon the use of the material. For example, compositions intended for administration to humans ordinarily must be highly purified in accordance with regulatory standards.

“A native sequence phage associated lytic enzyme” is a polypeptide having the same amino acid sequence as an enzyme derived from nature. This enzyme can be isolated from nature or can be produced by recombinant or synthetic means. The term “native sequence enzyme” specifically encompasses naturally occurring forms (e.g., alternatively spliced or modified forms) and naturally-occurring variants of the enzyme. In one embodiment of the disclosure, the native sequence enzyme is a mature or full-length polypeptide that is genetically coded for by a gene from a bacteriophage specific for a specific bacteria. Of course, a number of variants are possible and known, as acknowledged in publications such as Lopez et al., Microbial Drug Resistance 3: 199-211 (1997); Garcia et al., Gene 86: 81-88 (1990); Garcia et al., Proc. Nat]. Acad. Sci. USA 85: 914-918 (1988); Garcia et al., Proc. Natl. Acad. Sci. USA 85: 914-918 (1988); Garcia et al., Streptococcal Genetics (J. J. Ferretti and Curtis eds., 1987); Lopez et al., FEMS Microbiol. Lett. 100: 439-448 (1992); Romero et al., J. Bacteriol. 172: 5064-5070 (1990); Ronda et al., Eur. J. Biochem. 164: 621-624 (1987) and Sanchez et al., Gene 61: 13-19 (1987). The contents of each of these references, particularly the sequence listings and associated text that compares the sequences, including statements about sequence homologies, are specifically incorporated by reference in their entireties.

“A variant sequence phage associated lytic enzyme” means a functionally active lytic enzyme genetically coded for by a bacteriophage specific for a specific bacteria, as defined below, having at least 50%, 55%, 60%, 65%, 70%, 75%, 80%, 85%, 90%, 95%, 97%, 98%, 99%, or even at least 99.5% amino acid sequence identity with the sequences shown: in some of the figures. Of course a skilled artisan readily will recognize portions of this sequence that are associated with functionalities such as binding, and catalyzing a reaction. Accordingly, polypeptide sequences and nucleic acids that encode these sequences are contempplated that comprise at least 75%, 80%, 85%, 90%, 95%, 97%, 98%, 99% or more of each functional domain of some of the sequences. Such portions of the total sequence are very useful for diagnostics as well as therapeutics/prophylaxis. In fact, sequences as short as 5 amino acids long have utility as epitopic markers for the phage. More desirable, larger fragments or regions of protein having a size of at least 8, 9, 10, 12, 15 or 20 amino acids, and homologous sequences to these, have epitopic features :and may be used either as small peptides or as sections of larger proteins according to embodiments. Nucleic acids corresponding to these sequences also are contemplated.

Such phage associated lytic enzyme variants include, for instance, lytic enzyme polypeptides wherein one or more amino acid residues are added, or deleted at the N or C terminus of the sequence shown. In one embodiment one or more amino acids are substituted, deleted, and/or added to any position(s) in the sequence, or sequence portion. Ordinarily, a phage associated lytic enzyme will have at least about (e.g. exactly) 50%, 55%, 60%, 65%, 70%, 75%, amino acid sequence identity with native phage associated lytic enzyme sequences, more preferably at least about (e.g. exactly) 80%, 85%, 90%, 95%, 97%, 98%, 99% or 99.5% amino acid sequence identity. In other embodiments a phage associated lytic enzyme variant will have at least about 50% (e.g. exactly 50%), 55%, 60%, 65%, 70%, 75%, 80%, 85%, 90%, 95%, 97%, 98%, 99%, or even at least 99.5% amino acid sequence identity. with the sequences shown.

“Percent amino acid sequence identity” with respect to the phage associated lytic enzyme sequences identified herein is defined as the percentage of amino acid residues in a candidate sequence that are identical with the amino acid residues in the phage associated lytic enzyme sequence, after aligning the sequences in the same reading frame and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, such as using publicly available computer software such as blast software. Those skilled in the art can determine appropriate parameters for measuring alignment, including any algorithms needed to achieve maximal alignment over the whole length of the sequences being compared.

In each case, of course conservative amino acid substitutions also may be made simultaneously in determining percent amino acid sequence identity. For example, a 15 amino acid long region of protein may have 50%,.55%, 60%, 65%, 70%, 75%, 80%, 85%, 90%, 95%, 97%, 98%, or 99% sequence homology with a region of the sequences shown. At the same time, the 15 amino acid long region of the protein may also have up to 0.5%, 1%, 2%, 5%, 10%, 15%, 20%, 30%, 40%, 50%, 65%, 75%, or more amino acids replaced with conservative substitutions. Preferably the region will have fewer than 30%, 20%, 10% or even less conservative substitutions. The “percent amino acid sequence identity” calculation in such cases will be higher than the actual percent sequence identity when conservative amino acid substitutions have been made.

“Percent nucleic acid sequence identity” with respect to the phage associated lytic enzyme sequences identified herein is defined as the percentage of nucleotides in a candidate sequence that are identical with the nucleotides in the phage associated lytic enzyme sequence, after aligning the sequences and introducing gaps, if necessary, to achieve the maximum percent sequence identity. Alignment for purposes of determining percent nucleic acid sequence identity can be achieved in various ways that are within the scope of those skilled in the art, including but not limited to the use of publicly available computer software. Those skilled in the art can determine appropriate parameters for measuring aligrunent, including any algorithms needed to achieve maximal alignment over the full length of the sequences being compared.

“Polypeptide” refers to a molecule comprised of amino acids which correspond to those encoded by a polynucleotide sequence which is naturally occurring. The polypeptide may include conservative substitutions wherein the naturally occurring amino acid is replaced by one having similar properties, where such conservative substitutions do not alter the function of the polypeptide (see, for example, Lewin “Genes V” Oxford University Press Chapter 1, pp. 9-13 1994).

A “chimeric protein” or “fusion protein” comprises all or (preferably a biologically active) part of a polypeptide of the disclosure operably linked to a heterologous polypeptide. Chimeric proteins or peptides are produced, for example, by combining twd or more proteins having two or more active sites. Chimeric protein and peptides can act independently on the same or different molecules, and hence have a potential to treat two or more different bacterial infections at the same time. Chimeric proteins and peptides also are used to treat a bacterial infection by cleaving the cell wall in more than one location.

The term “operably linked” means that the polypeptide of the disclosure and the heterologous polypeptide are fused in-frame. The heterologous polypeptide can be fused to the N-terminus or C-termninus of the polypeptide of the disclosure. Chimeric proteins are produced enzymatically by chemical synthesis, or by recombinant DNA technology. A number of chimeric lytic enzymes have been produced and studied. Gene E-L, a chimeric lysis constructed from bacteriophages phi X174 and MS2 lysis proteins E and L, respectively, was subjected to internal deletions to create a series of new E-L clones with altered lysis or killing properties. The lyric activities of the parental genes E, L, E-L, and the internal truncated forms of E-L were investigated in this study to characterize the different lysis mechanism, based on differences in the architecture of the different membranes spanning domains. Electron microscopy and r*lease of marker enzymes for the cytoplasmic and periplasmic spaces revealed that two different lysis mechanisms can be distinguished depending on penetration of the proteins of either the inner membrane or the inner and outer membranes of the E. coli. FEMS Microbiol. Lett. Jul. 1, 1998, 164(1); 159-67 (incorporated herein by reference).

In another experiment, an active chimeric cell wall lytic enzyme (TSL) was constructed by fusing the region coding for the N-terminal half of the lactococcal phage Tuc2009 lysin and the region coding for the C-terminal domain of the major pneumococcal autolysin. The chimeric enzyme exhibited a glycosidase activity capable of hydrolysing choline-containing pneumococcal cell walls. One example of a useful fusion protein is a GST fusion protein in which the polypeptide of the disclosure is fused to the C-terminus of a GST sequence. Such a chimeric protein can facilitate the purification of a recombinant polypeptide of the disclosure.

In another embodiment, the chimeric protein or peptide contains a heterologous signal sequence at its N-terminus. For example, the native signal sequence of a polypeptide of the disclosure can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992, incorporated herein by reference). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

Another embodiment discloses an immunoglobulin fusion protein in which all or part of a polypeptide of the disclosure is fused to sequences derived from a member of the immunoglobulin protein family. An immunoglobulin fusion protein can be incorporated into a pharmaceutical composition and administered to a subject to inhibit an interaction between a ligand (soluble or membrane-bound) and a protein on the surface of a cell (receptor), to thereby suppress signal transduction in vivo. The immunoglobulin fusion protein can alter bioavailability of a cognate ligand of a polypeptide of the disclosure. Inhibition of ligand/receptor interaction may be useful therapeutically, both for treating bacterial-associated diseases and disorders for modulating (i.e. promoting or inhibiting) cell survival. Moreover, an immunoglobulin fusion protein of the disclosure can be used as an immunogen to produce antibodies directed against a polypeptide of the disclosure in a subject, to purify ligands and in screening assays to identify molecules which inhibit the interaction of receptors with ligands. Chimeric and fusion proteins and peptides of the disclosure can be produced by standard recombinant DNA techniques.

In another embodiment, the fusion gene can be synthesized by conventional techniques, including automated DNA synthesizers. Alternatively, PCR amplification of gene fragments can be carried out using anchor primers which give rise to complementary overhangs between two consecutive gene fragments which subsequently can be annealed and reamplified to generate a chimeric gene sequence (see, i.e., Ausubel et al., supra). Moreover, many expression vectors are commercially available that already encode a fusion moiety (i.e., a GST polypeptide). A nucleic acid encoding a polypeptide of the disclosure can be cloned into such an expression vector such that the fusion moiety is linked in-frame to the polypeptide of the disclosure.

As used herein, shuffled proteins or peptides, gene products, or peptides for more than one related phage protein or protein peptide fragments have been randomly cleaved and reassembled into a more active or specific protein. Shuffled oligonucleotides, peptides or peptide fragment molecules are selected or screened to identify a molecule having a desired functional property. This method is described, for example, in Stemmer, U.S. Pat. No. 6,132,970.(Method of shuffling polynucleotides); Kauffman, U.S. Pat. No. 5, 976,862 (Evolution via Condon-based Synthesis) and Huse, U.S. Pat. No. 5,808,022 (Direct Codon Synthesis). The contents of these patents are incorporated herein by reference. Shuffling is used to create a protein that is 10 to 100 fold more active than the template protein. The template protein is selected among different varieties of lysin or holin proteins. The shuffled protein or peptides constitute, for example, one or more binding domains and one or more catalytic domains. Each binding or catalytic domain is derived from the same or a different phage or phage protein. The shuffled domains are either oligonucleotide based molecules, as gene or gene products, that either alone or in combination with other genes or gene products are translatable into a peptide fragment, or they are peptide based molecules. Gene fragments include any molecules of DNA, RNA, DNA-RNA hybrid, antisense RNA, Ribozymes, ESTs, SNIPs and other oligonucleotide-based molecules that either alone or in combination with other molecules produce an oligonucleotide molecule capable or incapable of translation into a peptide.

As noted above, the present disclosure discusses the use of holin proteins. Holin proteins produce holes in the cell membrane. More specifically, holins form lethal membrane lesions. Like the lytic proteins, holin proteins are coded for and carried by a phage. In fact, it is quite common for the genetic code of the holin protein to be next to or even within the code for the phage lytic protein. Most holin protein sequences are short, and overall, hydrophobic in nature, with a highly hydrophilic carboxy-terminal domain. In many cases, the putative holin protein is encoded on a different reading frame within the enzymatically active domain of the phage. In other cases, holin protein is encoded on the DNA next or close to the DNA coding for the cell wall lytic protein. Holin proteins are frequently synthesized during the late stage of phage infection and found in the cytoplasmic membrane where they cause membrane lesions.

Holins can be grouped into two general classes based on primary structure analysis. Class I holins are usually 95 residues or longer and may have three potential transmembrane domains. Class II holins are usually smaller, at approximately 65-95 residues, with the distribution of charged and hydrophobic residues indicating two TM domains (Young, et al. Trends in Microbiology v. 8, No. 4, March 2000). At least for the phages of gram-positive hosts, however, the dual-component lysis system may not be universal. Although the presence of holins has been shown or suggested for several phages, no genes have yet been found encoding putative holins for all phages. Holins have been shown to be present in several bacteria, including, for example, lactococcal bacteriophage Tuc2009, lactococcal NLC3, pneumococcal bacteriophage EJ-1, Lactobacillus gasseri bacteriophage Nadh, Staphylococcus aureus bacteriophage Twort, Listeria monocytogenes bacteriophages, pneumococcal phage Cp-1, Bacillus subtillis phage M29, Lactobacillus delbrueckki bacteriophage LL-H lysin, and bacteriophage N11 of Staphyloccous aureus. (Loessner, et al., Journal of Bacteriology, August 1999, p. 4452-4460).

The modified or altered form of the protein or peptides and peptide fragments, as disclosed herein, includes protein or peptides and peptide fragments that are chemically synthesized or prepared by recombinant DNA techniques, or both. These techniques include, for example, chimerization and shuffling. When the protein or peptide is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

In one embodiment of the disclosure, a signal sequence of a polypeptide can facilitate transmembrane movement of the protein and peptides and peptide fragments of the disclosure to and from mucous membranes, as well as by facilitating secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the disclosure can pertain to the described polypeptides having a signal sequence, as well as to the signal sequence itself and to the polypeptide in the absence of the signal sequence (i.e., the cleavage products). In one embodiment, a nucleic acid sequence encoding a signal sequence of the disclosure can be operably linked in an expression vector to a protein of interest, such as a protein which is ordinarily not secreted or is otherwise difficult to isolate. The signal sequence directs secretion of the protein, such as from an eukaryotic host into which the expression vector is transformed, and the signal sequence is subsequently or concurrently cleaved. The protein can then be readily purified from the extracellular medium by art recognized methods. Alternatively, the signal sequence can be linked to a protein of interest using a sequence which facilitates purification, such as with a GST domain.

In another embodiment, a signal sequence can be used to identify regulatory sequences, i.e., promoters, enhancers, repressors. Since signal sequences are the most amino-terminal sequences of a peptide, it is expected that the nucleic acids which flank the signal sequence on its amino-terminal side will be regulatory sequences that affect transcription. Thus, a nucleotide sequence which encodes all or a portion of a signal sequence can be used as a probe to identify and isolate the signal sequence and its flanking region, and this flanking region can be studied to identify regulatory elements therein. The present disclosure also pertains to other variants of the polypeptides of the disclosure. Such variants have an altered amino acid sequence which can function as either agonists (mimetics) or as antagonists. Variants can be generated by mutagenesis, i.e., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein. Variants of a protein of the disclosure which function as either agonists (mimetics) or as antagonists can be identified by screening combinatorial libraries of mutants, i.e., truncation mutants, of the protein of the disclosure for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (i.e., for phage display). There are a variety of methods which can be used to produce libraries of potential variants of the polypeptides of the disclosure from a degenerate oligonucleotide sequence. Methods for synthesizing degenerate oligonucleotides are known in the art (see, i.e., Narang (1983) Tetrahedron 39:3; Itakura et al. (1984) Annu. Rev. Biochem 53:323; Itakura et al. (1984) Science 198:1056; Ike et al. (1983) Nucleic Acid Res. 11:477, all herein incorporated by reference).

In addition, libraries of fragments of the coding sequence of a polypeptide of the disclosure can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with SI nuclease, and ligating the resulting fragment library into an expression vector. By this method, an expression library can be derived which encodes N-terminal and internal fragments of various sizes of the protein of interest. Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the disclosure (Arkin and Yourvan (1992) Proc. Natl. Acad. Sci. USA 89:7811-7815; Delgrave et al. (1993) Protein Engineering 6(3):327-331).

Immunologically active portions of a protein or peptide fragment include regions that bind to antibodies that recognize the phage enzyme. In this context, the smallest portion of a protein (or nucleic acid that encodes the protein) according to embodiments is an epitope that is recognizable as specific for the phage that makes the lysin protein. Accordingly, the smallest polypeptide (and associated nucleic acid that encodes the polypeptide) that can be expected to bind antibody and is useful for some embodiments may be 8, 9, 10, 11, 12, 13, 15, 20, 25,30, 35, 40,45, 50, 55, 60, 65, 75, 85, or 100 amino acids long. Although small sequences as short as 8, 9, 10, 11, 12 or 15 amino acids long reliably comprise enough structure to act as epitopes, shorter sequences of 5, 6, or 7 amino acids long can exhibit epitopic structure in some conditions and have value in an embodiment. Thus, the smallest portion of the protein or nucleic acid sequence described by specific sequences includes polypeptides as small as 5, 6, 7, 8, 9, or 10 amino acids long.

Homologous proteins and nucleic acids can be prepared that share functionality with such small proteins and/or nucleic acids (or protein and/or nucleic acid regions of larger molecules) as will be appreciated by a skilled artisan. Such small molecules and short regions of larger molecules, that may be homologous specifically are intended as embodiments. Preferably the homology of such valuable regions is at least 50%, 65%, 75%, 85%, and more preferably at least 90%, 95%, 97%, 98%, or at least 99% compared to the specific sequences. These percent homology values do not include alterations due to conservative amino acid substitutions.

Of course, an epitope as described herein may be used to generate an antibody and also can be used to detect binding to molecules that recognize the lysin protein. Another embodiment is a molecule such as an antibody or other specific binder that may be created through use of an epitope such as by regular immunization or by a phase display approach where an epitope can be used to screen a library of potential binders. Such molecules recognize one or more epitopes of lysin protein or a nucleic acid that encodes lysin protein. An antibody that recognizes an epitope may be a monoclonal antibody, a humanized antibody, or a portion of an antibody protein. Desirably the molecule that recognizes an epitope has a specific binding for that epitope which is at least 10 times as strong as the molecule has for serum albumin. Specific binding can be measured as affinity (Kin). More desirably the specific binding is at least 10², 10³, 10⁴, 10⁵, 10⁶, 10⁸, or even higher than that for serum albumin under the same conditions.

In a desirable embodiment the antibody or antibody fragment is in a form useful for detecting the presence of the lysin protein. A variety of forms and methods for their synthesis are kno The antibody may be conjugated (covalently complexed) with a reporter molecule or atom such as a fluor, an enzyme that creates an optical signal, a chemilumiphore, a microparticle, or a radioactive atom. The antibody or antibody fragment may be synthesized in vivo, after immunization of an animal, for example, The antibody or antibody fragment may be synthesized via cell culture after genetic recombination. The antibody or antibody fragment may be prepared by a combination of cell synthesis and chemical modification.

Biologically active portions of a protein or peptide fragment of the embodiments, as described herein, include polypeptides comprising amino acid sequences sufficiently identical to or derived from the amino acid sequence of the phage protein of the disclosure, which include fewer amino acids than the fill length protein of the phage protein and exhibit at least one activity of the corresponding full-length protein. Typically, biologically active portions comprise a domain or motif with at least one activity of the corresponding protein. A biologically active portion of a protein or protein fragment of the disclosure can be a polypeptide which is, for example, 10, 25, 50, 100 less or more amino acids in length. Moreover, other biologically active portions, in which other regions of the protein are deleted, or added can be prepared by recombinant techniques and evaluated for one or more of the functional activities of the native form of a polypeptide of the embodiments.

A large variety of isolated cDNA sequences that encode phage associated lysing enzymes and partial sequences that hybridize with such gene sequences are useful for recombinant production of the lysing enzyme. Representative nucleic acid sequences in this context are the sequences shown in the figures and sequences that hybridize, under stringent conditions, with complementary sequences of the DNA from those sequences. Still further variants of these sequences and sequences of nucleic acids that hybridize with those shown in the figures also are contemplated for use in production of lysing enzymes according to the disclosure, including natural variants that may be obtained.

Many of the contemplated variant DNA molecules include those created by standard DNA mutagenesis techniques, such as M13 primer mutagenesis. Details of these techniques are provided in Sambrook et al. (1989) In Molecular Cloning: A Laboratory Manual, Cold Spring Harbor, N.Y. (incorporated herein by reference). By the use of such techniques, variants may be created which differ in minor ways from those disclosed. DNA molecules and nucleotide sequences which are derivatives of those specifically disclosed herein and which differ from those disclosed by the deletion, addition or substitution of nucleotides while still encoding a protein which possesses the functional characteristic of the BSMR protein are contemplated by the disclosure. Also included are one small DNA molecules which are derived from the disclosed DNA molecules. Such small DNA molecules include oligonucleotides suitable for use as hybridization probes or polymerase chain reaction (PCR) primers. As such, these small DNA molecules will comprise at least a segment of a lytic enzyme genetically coded for by a bacteriophage specific for a specific bacteria and, for the purposes of PCR, will comprise at least a 10-15 nucleotide sequence and, more preferably, a 15-30 nucleotide sequence of the gene. DNA molecules and nucleotide sequences which are derived from the disclosed DNA molecules as described above may also be defined as DNA sequences which hybridize under stringent conditions to the DNA sequences disclosed, or fragments thereof.

Hybridization conditions corresponding to particular degrees of stringency vary depending upon the nature of the hybridization method of choice and the composition and length of the hybridizing DNA used. Generally, the temperature of hybridization and the ionic strength (especially the sodium ion concentration) of the hybridization buffer will determine the stringency of hybridization. Calculations regarding hybridization conditions required for attaining particular degrees of stringency are discussed by Sambrook et al. (1989), In Molecular Cloning: A Laboratory Manual, Cold Spring Harbor, N.Y., chapters 9 and 11, (herein incorporated by reference).

An example of such calculation is as follows. A hybridization experiment may be performed by hybridization of a DNA molecule (for example, a natural variation of the lytic enzyme genetically coded for by a bacteriophage specific for Bacillus anthracis) to a target DNA molecule. A target DNA may be, for example, the corresponding cDNA which has been electrophoresed in an agarose gel and transferred to a nitrocellulose membrane by Southern blotting (Southern (1975). J. Mol. Biol. 98:503), a technique well known in the art and described in Sambrook et al. (1989) In Molecular Cloning: A Laboratory Manual, Cold Spring Harbor, N.Y. (incorporated herein by reference). Hybridization with a target probe labeled with isotopic P (32) labeled-dCTP is carried out in a solution of high ionic strength such as 6 times SSC at a temperature that is 20-25 degrees Celsius below the melting temperature, Tm, (described infra). For such Southern hybridization experiments where the target DNA molecule on the Southern blot contains 10 ng of DNA or more, hybridization is carried out for 6-8 hours using 1-2 ng/ml radiolabeled probe (of specific activity equal to 109 CPM/mug or greater). Following hybridization, the nitrocellulose filter is washed to remove background hybridization. The washing conditions are as stringent as possible to remove background hybridization while retaining a specific hybridization signal. The term “Tm” represents the temperature above which, under the prevailing ionic conditions, the radiolabeled probe molecule will not hybridize to its target DNA molecule.

The Tm of such a hybrid molecule may be estimated from the following equation: Tm=81.5 degrees C−16.6(log10 of sodium ion concentration)+0.41(% G+C)−0.63(% fonnamide)-(600/I) where I=the length of the hybrid in base pairs. This equation is valid for concentrations of sodium ion in the range of 0.01M to 0.4M, and it is less accurate for calculations of Tm in solutions of higher sodium ion concentration (Bolton and McCarthy (1962). Proc. Natl. Acad. Sci. USA 48:1390) (incorporated herein by reference). The equation also is valid for DNA having G+C contents within 30% to 75%, and also applies to hybrids greater than 100 nucleotides in length. The behavior of oligonucleotide probes is described in detail in Ch. 11 of Sambrook et al. (1989), In Molecular Cloning: A Laboratory Manual, Cold Spring Harbor, N.Y. (incorporated herein by reference). The preferred exemplified conditions described here are particularly contemplated for use in selecting variations of the lytic gene.

Thus, by way of example, of a 150 base pair DNA probe derived from the first 150 base pairs of the open reading frame of a cDNA having a % GC=45%, a calculation of hybridization conditions required to give particular stringencies may be made as follows:

Assuming that the filter will be washed in 0.3×SSC solution following hybridization, sodium ion=0.045M; % GC=45%; Formamide concentration=0 1=150 base pairs (see equation in Sambrook et al.) and so Tm=74.4 degrees C. The Tm of double-stranded DNA decreases by 1-1.5 degrees C with every 1% decrease in homology (Bonner et al. (1973). J. Mol. Biol. 81:123). Therefore, for this given example, washing the filter in 0.3 times SSC at 59.4-64.4 degrees C. will produce a stringency of hybridization equivalent to 90%; DNA molecules with more than 10% sequence variation relative to the target BSMR cDNA will not hybridize. Alternatively, washing the hybridized filter in 0.3 times SSC at a temperature of 65.4-68.4 degrees C. will yield a hybridization stringency of 94%; DNA molecules with more than 6% sequence variation relative to the target BSMR cDNA molecule will not hybridize. The above example is given entirely by way of theoretical illustration. One skilled in the art will appreciate that other hybridization techniques may be utilized and that variations in experimental conditions will necessitate alternative calculations for stringency.

In preferred embodiments of the present disclosure, stringent conditions may be defined as those under which DNA molecules with more than 25% sequence variation (also termed “mismatch”) will not hybridize. In a more preferred embodiment, stringent conditions are those under which DNA molecules with more than 15% mismatch will not hybridize, and more preferably still, stringent conditions are those under which DNA sequences with more than 10% mismatch will not hybridize. Preferably, stringent conditions are those under which DNA sequences with more than 6% mismatch will not hybridize.

The degeneracy of the genetic code further widens the scope of the embodiments as it enables major variations in the nucleotide sequence of a DNA molecule while maintaining the amino acid sequence of the encoded protein. For example, a representative amino acid residue is alanine. This may be encoded in the cDNA by the nucleotide codon triplet GCT. Because of the degeneracy of the genetic code, three other nucleotide codon triplets—GCT, GCC and GCA—also code for alanine. Thus, the nucleotide sequence of the gene could be changed at this position to any of these three codons without affecting the amino acid composition of the encoded protein or the characteristics of the protein. Tie genetic code and variations in nucleotide codons for particular amino acids are well known to the skilled artisan. Based upon the degeneracy of the genetic code, variant DNA molecules may be derived from the cDNA molecules disclosed herein using standard DNA mutagenesis techniques as described above, or by synthesis of DNA sequences. DNA sequences which do not hybridize under stringent conditions to the cDNA sequences disclosed by virtue of sequence variation based on the degeneracy of the genetic code are herein comprehended by this disclosure.

One skilled in the art will recognize that the DNA mutagenesis techniques described here can produce a wide variety of DNA molecules that code for a bacteriophage lysin specific for a specific bacteria yet that maintain the essential characteristics of the lytic protein. Newly derived proteins may also be selected in order to obtain variations on the characteristic of the lytic protein, as will be more fully described below. Such derivatives include those with variations in amino acid sequence including minor deletions, additions and substitutions.

While the site for introducing an amino acid sequence variation is predetermined, the mutation per se does not need to be predetermined. For example, in order to optimize the performance of a mutation at a given site, random mutagenesis may be conducted at the target codon or region and the expressed protein variants screened for the optimal combination of desired activity. Techniques for making substitution mutations at predetermined sites in DNA having a known sequence as described above are well known.

Amino acid substitutions are typically or single residues; insertions usually will be on the order of about from 1 to 10 amino acid residues; and deletions will range about from 1 to 30 residues. Deletions or insertions may be in single form, but preferably are made in adjacent pairs, i.e., a deletion of 2 residues or insertion of 2 residues. Substitutions, deletions, insertions or any combination thereof may be combined to arrive at a final construct. Obviously, the mutations that are made in the DNA encoding the protein must not place the sequence out of reading frame and preferably will not create complementary regions that could produce secondary mRNA structure (EP 75,444A). Substitutional variants are those in which at least one residue in the amino acid sequence has been removed and a different residue inserted in its place. Such substitutions may be made in accordance with the following Table 1 when it is desired to finely modulate the characteristics of the protein. Table 1 shows amino acids which may be substituted for an original amino acid in a protein and which are regarded as conservative substitutions.

TABLE 1
Original Residue
Conservative Substitutions

| | |
|---|---|
| Ala | ser |
| Arg | lys |
| Asn | gln, his |
| Asp | glu |
| Cys | ser |
| Gln | asn |
| Glu | asp |
| Gly | pro |
| His | asn; gln |
| Ile | leu, val |
| Leu | ile; val |
| Lys | arg; gln; glu |
| Met | leu; ile |
| Phe | met; leu; tyr |
| Ser | thr |
| Thr | ser |
| Trp | tyr |
| Tyr | trp; phe |
| Val | ile; leu |

Substantial changes in function or immunological identity are made by selecting substitutions that are less conservative than in Table 1, i.e., selecting residues that differ more significantly in their effect on maintaining: (a) the structure of the polypeptide backbone in the area of the substitution, for example, as a sheet or helical conformation; (b) the charge or hydrophobicity of the molecule at the target site; or (c) the bulk of the side chain. The substitutions which in general are expected to produce the greatest changes in protein properties will be those in which: (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histadyl, is substituted for (or by) an electronegative residue, e.g., glutamyl or aspartyl; or (d) a residue having a bulky side chain, e.g., phenylalanine, is substituted for (or by) one not having a side chain, e.g., glycine.

The effects of these amino acid substitutions or deletions or additions may be assessed for derivatives of the lytic protein by analyzing the ability of the derivative proteins to complement the sensitivity to DNA cross-linking agents exhibited by phages in infected bacteria hosts. These assays may be performed by transfecting DNA molecules encoding the derivative proteins into the bacteria as described above.

Having herein provided nucleotide sequences that code for lytic enzyme genetically coded for by a bacteriophage specific for a specific bacteria and fragments of that enzyme, correspondingly provided are the complementary DNA strands of the cDNA molecule and DNA molecules which hybridize under stringent conditions to the lytic enzyme cDNA molecule or its complementary strand. Such hybridizing molecules include DNA molecules differing only by minor sequence changes, including nucleotide substitutions, deletions and additions. Also contemplated -by this disclosure are isolated oligonucleotides comprising at least a segment of the cDNA molecule or its complementary strand, such as oligonucleotides which may be employed as effective DNA hybridization probes or primers useful in the polymerase chain reaction. Hybridizing DNA molecules and variants on the lytic enzyme cDNA may readily be created by standard molecular biology techniques.

The detection of specific DNA mutations may be achieved by methods such as hybridization using specific oligonucleotides (Wallace et al. (1986). Cold Spring Harbor Symp. Quant. Biol. 51:257-261), direct DNA sequencing (Church and Gilbert (1988). Proc. Natl. Acad. Sci. USA 81:1991-1995), the use of restriction enzymes (Flavell et al. (1978). Cell 15:25), discrimination on the basis of electrophoretic mobility in gels with denaturing reagent (Myers and Maniatis (1986). Cold Spring Harbor Symp. Quant. Biol. 51:275-284), RNase protection (Myers et al. (1985). Science 230:1242), chemical cleavage (Cotton et al. (1985). Proc. Natl. Acad. Sci. USA 85:4397-4401) (incorporated herein by reference), and the ligase-mediated detection procedure (Landegren et al., 1988).

Oligonucleotides specific to normal or mutant sequences are chemically synthesized using commercially available machines, labeled radioactively with isotopes (such as .sup.32 P) or non-radioactively (with tags such as biotin (Ward and Langer et al. Proc. Natl. Acad. Sci. USA 78:6633-6657 1981) (incorporated herein by reference), and hybridized to individual DNA samples immobilized on membranes or other solid supports by dot-blot or transfer from gels after electrophoresis. The presence or absence of these specific sequences are visualized by methods such as autoradiography or fluorometric or colorimetric reactions (Gebeyehu et al. Nucleic Acids Res. 15:4513-4534 1987) (incorporated herein by reference).

Sequence differences between normal and mutant forms of the gene may also be revealed by the direct DNA sequencing method of Church and Gilbert (1988) (incorporated herein by reference). Cloned DNA segments may be used as probes to detect specific DNA segments. The sensitivity of this method is greatly enhanced when combined with PCR (Stoflet et al. Science 239:491-494, 1988) (incorporated herein by reference). In this approach, a sequencing primer which lies within the amplified sequence is used with double-stranded PCR product or single-stranded template generated by a modified PCR. The sequence determination is performed by conventional procedures with radiolabeled nucleotides or by automatic sequencing procedures with fluorescent tags. Such sequences are useful for production of lytic enzymes according to embodiments of the disclosure.

Additional objects and advantages embodiments found in the disclosure will be set forth in the description which follows, and in part will be obvious from the description, or may be learned by practice of the embodiments.! The objects and advantages of the disclosure may be realized and obtained by means of the instrumentalities and combinations particularly pointed out in the appended claims.

### BRIEF DESCRIPTION OF THE DRAWINGS

FIG. 1 is an electron micrograph of group A streptococci treated with lysin showing the collapse of the cell wall and the cell contents pouring out;

FIG. 2 is a graph for the killing of S. pneumoniae (#DCC 1490) serotype 14 with PAL at various dilutions;

FIG. 3 is a graph showing the decrease of bacterial titer within 30 seconds after addition of 100 U Pal phage enzyme;

FIG. 4 is a series of graphs showing the decrease of the Bacterial titer with 30 seconds after the addition of 100, 1,000, and 10,000 U Pal lytic Enzyme;

FIG. 5 is a series of graphs showing the decrease of bacterial titer within 30 seconds after addition of different amounts of U Pal.

FIG. 6 depicts a histogram showing Group A Streptococci, Group B to N Streptococci, and oral Streptococci, with die optical density of different strains of bacteria at OD650/min. measured against different concentration of Pal enzyme; and

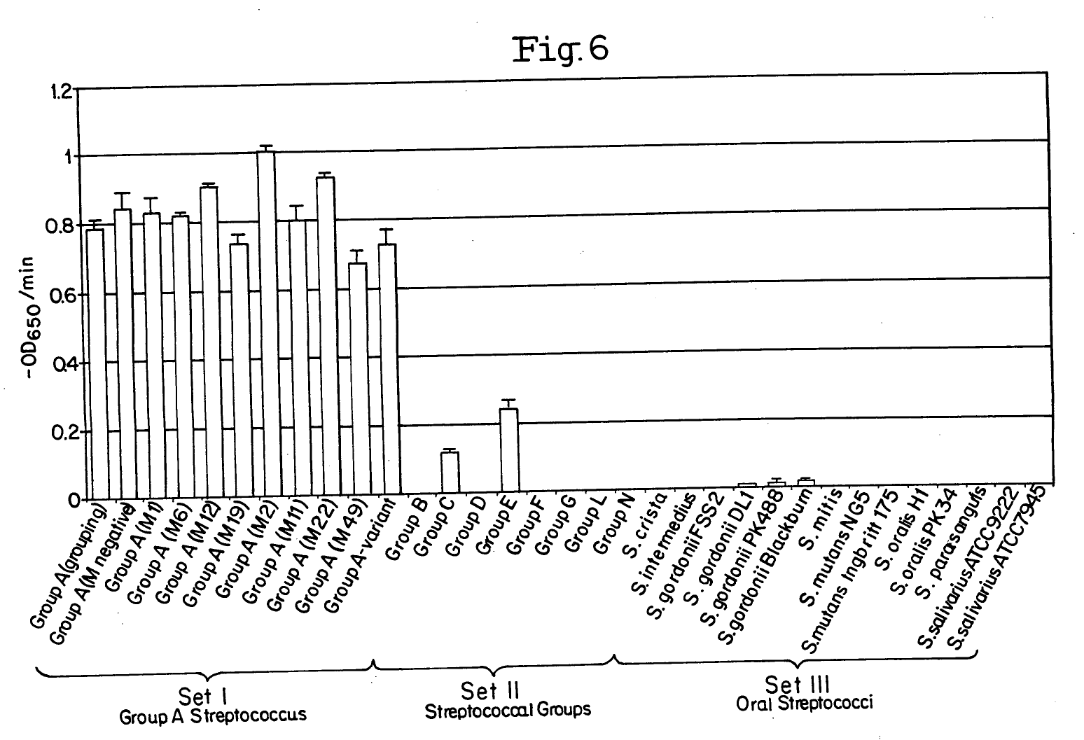

FIG. 7 shows polyacrylamide gel showing molecular weight of a lysin peptide;

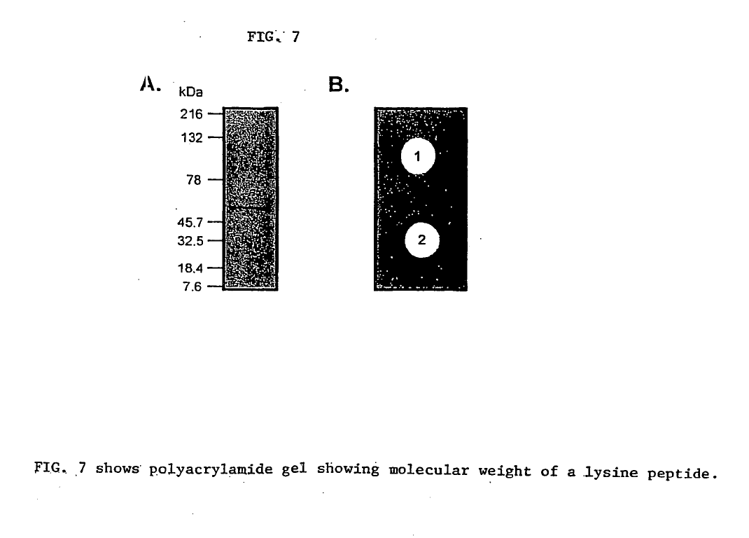

### DETAILED DESCRIPTION OF THE DISCLOSURE

This disclosure, discloses prophylactic and therapeutic compositions containing as an active ingredient, one or more bacteria-associated phage proteins or protein peptides fragments, including isozymes, analogs, or variants of phage enzymes or phage peptides and peptide fragments thereof in a natural or modified form as active drugs and the method in therapeutic, diagnostic, and drug screening of use of such compositions. The bacteria-associated phage proteins, include a variety of bacteria-specific phage lytic and holin proteins that are derived from one or several bacterial species.

Bacteriophage lytic proteins specifically cleave bonds that are present in the peptidoglycan of bacterial cells. Since the peptidoglycan is highly conserved among all bacteria, there are only a few bonds to be cleaved to disrupt the cell wall. Proteins having the ability to hydrolyze components of a bacterial peptidoglycan fall into one of four categories:

1. N-acetylmuramoyl-L-alanine amidases (E.C. 3.5.1.28)—These proteins hydrolyze the link between N-acetylmuramoyl residues and L-amino acid residues in certain bacterial cell-wall glycopeptides.

Streptococcal lysin belongs to this family of lytic proteins. Of the 27 sequenced amidases, only the five highlighted are of bacteriophage origin. The rest are autolysins of bacterial origin.

2. Lysozyme. (EC 3.2.1.17), also known as muramidases. This protein hydrolyses the 1,4-beta-linkages between N-acetyl-D-glucosamine and N-acetylmuramic acid in peptidoglycan heteropolymers of the prokaryotes cell walls.

Of the 94 known sequences, 15 are encoded by bacteriophages.

3. Beta 1,4 N-acetyl-D-glucosaminidase (EC 3.2.1.14), also known as chitinase or chitodextrinase. Hydrolysis of the 1 4-beta-linkages of -acetyl-D-glucosamine polymers of chitin. These proteins are found primarily in the plant kingdom, although some are found in bacteria. None of the 104 known proteins are encoded by bacteriophages. However, many of these proteins that are produced by bacteria also possess lysozyme activity, and are usually classified with the other lysozymes.

4. Endopeptidase that cleaves the cross bridge of the peptidoglycan. The only known endopeptidase to be characterized extensively which acts on the peptidoglycan is lysostaphin (EC 3.4.24.75). This is a metalloprotease that hydrolyses the -Gly-1-Glybond in the pentaglycine inter-peptide link joining staphylococcal cell wall peptidoglycans. This protein is found in several streptococcal species, but it is not encoded by bacteriophages. The only reported phage encoded endopeptidase that acts on the peptidoglycan is from a Pseudomonas phi 6 phage.

The majority of reported phage proteins are either muramidases or amidases. Fischetti et al (1974) reported that the C1 streptococcal phage lysine protein was an amidase. Garcia et al (1987, 1990) reported that the CP-1 lysin from an S. pheumoniae phage was a muramidase. Caldentey and Bamford (1992) reported that a lytic protein from the phi 6 Pseudomonas phage was an endopeptidase, splitting the peptide bridge formed by meso-diaminopimilic acid and D-alanine. The E. coli T1 and T6 phage lytic proteins are amidases as is the lytic protein from Listeria phage (ply) (Loessner et al 1996).

Infection of the Hemophilus bacteria by Bacteriophage HP1 (a member of the P2-like phage family with strong similarities to coliphages P2 and 186, and some similarity to the retrophage Ec67) produces a lytic protein capable of lysing the bacteria. The lytic protein for Streptococcus pneumoniae, previously identified as a -acetyl-muramoyl-L-alanine amidase, is produced by infecting Streptococcus pheumoniae with the Pal bacteriophage. The therapeutic composition contains either or both of the lytic proteins produced by these two bacteria, and also contains other lytic proteins from other bacteria.

These proteins are specifically effective in prophylactic and therapeutic treatment of bacterial infections of the upper respiratory tract. The infection can be prophylactically or therapeutically treated with a composition comprising an effective amount of at least one lytic protein genetically coded for by a bacteriophage specific for that bacteria, and an application means for delivering the lytic protein to the cite of the infection, as for example, tile mouth, throat, or nasal passage.

For example, Streptococcus group A which produces what-is commonly known as “strep” throat, is treated prophylactically and therapeutically by the application of lytic proteins specific for Streptococcus group A. Group C Streptococci infected with a C1 bacteriophage produce a lytic protein that is specific for the lysing of Streptococcus group A. The composition used for the prophylactic and therapeutic treatment of a strep infection includes, for example, one or more lytic proteins and a pharmaceutically acceptable carrier for the mucosal lining of the oral and nasal cavity, such that the protein reaches the mucosal lining.

Bacteriophages

There are a large number of phages which will attach to specific bacteria and produce enzymes which will lyse that particular bacteria. The following are a list of bacteriophages and bacteria for which they are specific and which can be treated when infecting a body:

```
Actinomycetes
  Actinomyces israelii
Agrobacterium
Alcaligenes
Bacillus
  Bacillus anthracis
Bacteroides
  Bacteroides fragilis
Bartonella
  Bartonella bacilliformis
  Bartonella henselae
Bdellovibrio
Bordetella
  Bordetella pertussis
Borrelia
  Borrelia burdorferi
  Borrelia recurrentis
Brucella
  Brucella abortus
  Brucella melitensis
  Brucella suis
Burkholderia
Calymmatobacterium
  Calymmatobacterium donovani
Campylobacter
  Campylobacter fetus
  Campylobacter jejuni
Caulobacter
Clostridium
  Clostridium botulinum
  Clostridium difficile
  Clostridium perfringens
  Clostridium septicum
  Clostridium tetani
Corynebacteria
  Corynebacterium diptheriae
Coryneforms
Cyanobacteria
Enterobacteria
  Enterobacter (Aerobacter) aerogenes
Escherschia coli
Francisella
  Francisella tularensis
Haemophilus
  Haemophilus ducreyi
  Haemophilus influenzae
Klebsiella
  Klebisiella ozaenae
  Klebsiella pneumoniae
  Klebsiella rhinoscleromatis
Lactobacillus
Lactoctococcus
Legionella
  Legionella pneumophila
Leptospira
Listeria
  Listeria monocytogenes
Micrococcus
Mollicutes
Mycobacteria
  Mycobacterium avium
  Mycobacterium bovis
  Mycobacterium intracellulare
  Mycobacterium kansasii
  Mycobacterium leprae
  Mycobacterium tuberculosis
  Mycobacterium ulcerans
Myxococcus
Neisseria
  Neisseria gonorrhoeae
  Neisseria meningitidis
Pasteurella
Pneumococci
Proteus
  Proteus mirabilis
  Proteus morgagni
Pseudomonas
  Pseudomonas aeruginosa
  Pseudomonas mallei
  Pseudomonas pseudomalli
Rhizobium
Salmonella
  Salmonella typhi
  Salmonella typhimurium
Serratia
  Serratia marcescens
Shigella
Spirillum
  Spirillum minus
Spirochete
Spiroplasma
Staphylococci
  Staphylococcus aureus
Streptobacillus
  Streptobacillus moniliformis
Streptococci
  Streptococcus pyogenes
  Streptococcus pneumoniae
Treponema
  Treponema carateum
  Treponema pallidum
  Treponema pertenue
Vibrio
  Vibrio cholerae
Xanthomonas
Yersinia
  Yersinia enterocolitica
  Yersinia pestis
```

Various phages which can be used to infect these bacteria and create the lytic enzyme include:

| BACTERIA | PHAGE(S) |
|---|---|
| Acholeplasma | BN1, MV-L3, (syn = MVL3), MVL51, MVL52, MV-L59, |
| | MV-L60, 03cl, 011clr, 10tur, 143tur, 179tur, 182tur, |
| | 1304clr, MV-L1 |
| Achromobacter | OXN-36P, NN-Achromobacter (1) |
| Acinetobacter | A31, A33, A34, A36, A37, BP1, B₉GP, P78, 56. 142, 205, |
| | E4, E5, HP1, 102, 106, 133, A1, A3/2, A9, A10/45, BS46, |
| | E1, E2, E7, E14, G4, HP2, HP3, HP4, 20, 59, 73, 103, 104, |
| | 108, 138, 141, 143, 196, 204, 206, E6, E8, E9, E13, E15, 1, |
| | 11, 66 |
| Actinobacillus | Aaφ23, Aaφ76, Aaφ97, Aaφ99, Aaφ247, PAA24, PAA84, |
| | φAa17, NN-Actinobacillus (1) PAA17, PAA23, NN- |
| | Actinobacillus (2) |
| Actinomycetes | Av-1, Av-2, Av-3, BF307, CT1, CT2, CT3, CT4, CT8, CT6, |
| | CT7, 1281 |
| Aeromonas | AA-1, 29, 37, 43, 51, 59.1, Aeh1, F, PM2, 1, 25, 31, |
| | 40RR2.8t, (syn = 44R), (syn = 44RR_{2.8}t), 65, Aeh2, N, PM1, |
| | TP446, 3, 4, 11, 13, 29, 31, 32, 37, 43, 43-10T, 51, 54, 55R. |
| | 1, 56, 56RR2, 57, 58, 59.1, 60, 63, PM3, PM4, PM5, PM6 |
| Altermonas | PM2 |
| Bacillus | 4 (B. megaterium), 4 (B. sphaericus) ale1, AR1, AR2, AR3, |
| | AR7, AR9, Bace-11, (syn = 11), Bastille, BL1, BL2, BL3, |
| | BL4, BL5, BL6, BL8, BL9, BP124, BS28, BS80, Ch, CP- |
| | 51, CP-54, D-5, dar1, den1, DP-7, ent2, FoS₁, FoS₂, FS₄, |
| | FS₆, FS₇, G, gal1, gamma, GE1, GF-2, GS₁, GT-1, GT-2, |
| | GT-3, GT-4, GT-5, GT-6, GT-7, GV-6, g15, I9, I10, IS₁, K, |
| | MP9, MP13, MP21, MP23, MP24, MP28, MP29, MP30, |
| | MP32, MP34, MP36, MP37, MP39, MP40, MP41, MP43, |
| | MP44, MP45, MP47, MP50, NLP-1, No. 1, N17, N19, PBS1, |
| | PK1, PMB1, PMB12, PMJ1, S, SPO1, SP3, SP5, SP6, SP7, |
| | SP8, SP9, SP10, SP-15, SP50, (syn = SP-50), SP82, SST, |
| | sub1, SW, Tg8, Tg12, Tg13, Tg14, thu1, thu4, thu5, Tin4, |
| | Tin23, TP-13, TP33, TP50, TSP-1, type V, type VI, V, Vx, |
| | β22, φe, φNR2, φ25, φ63, 1, 1, 2, 2C, 3NT, 4, 5, 6, 7, 8, 9, |
| | 10, 12, 12, 17, 18, 19, 21, 138, III, AR13, BPP-10, BS32, |
| | BS107, B1, B2, GA-1, GP-10, GV-3, GV-5, g8, MP20, |
| | MP27, MP49, Nf, PP5, PP6, SF5, Tg18, TP-1, Versailles, |
| | φ15, φ29, 1-97, 837/IV, NN-Bacillus (1) A, aiz1, Al-K-I, B, |
| | BCJA1, BC1, BC2, BLL1, BL1, BP142, BSL1, BSL2, BS1, |
| | BS3, BS8, BS15, BS18, BS22, BS26, BS28, BS31, BS104, |
| | BS105, BS106, BTB, B1715V1, C, CK-1, Co11, Cor1, CP- |
| | 53, CS-1, CS₁, D, D, D, D5, ent1, FP8, FP9, FS₁, FS₂, |
| | FS₃, FS₅, FS₈, FS₉, G, GH8, GT8, GV-1, GV-2, GT-4, |
| | g3, g12, g13, g14, g16, g17, g21, g23, g24, g29, H2, |
| | ken1, KK-88, Kum1, Kyu1, J7W-1, LP52, (syn = LP-52), |
| | L₇, Mex1, MJ-1, mor2, MP-7, MP10, MP12, MP14, |
| | MP15, Neol, N°2, N5, N6P, PBC1, PBLA, PBP1, P2, |
| | S-a, SF2, SF6, Sha1, Sil1, SPO2, (syn = ΦSPP1), SPβ, |
| | STI, ST₁, SU-11, t, Tb1, Tb2, Tb5, Tb10, Tb26, Tb51, |
| | Tb53, Tb55, Tb77, Tb97, Tb99, Tb560, Tb595, Td8, |
| | Td6, Td15, Tg1, Tg4, Tg6, Tg7, Tg9, Tg10, Tg11, |
| | Tg13, Tg15, Tg21, Tin1, Tin7, Tin8, Tin13, Tm3, Toc1, |
| | Tog1, tol1, TP-1, TP-10ᵥᵢᵣ, TP-15c, TP-16c, TP-17c, |
| | TP-19, TP35, TP51, TP-84, Tt4, Tt6, type A, type B, |
| | type C, type D, type E, Tφ3, VA-9, W, wx23, wx26, |
| | Yun1, α, γ, ρ11,, φmed-2, φT, φμ-4, φ3T, φ75, φ105, |
| | (syn = φ105), 1A, 1B, 1-97A, 1-97B, 2, 2, 3, 3, 3, 5, 12, |
| | 14, 20, 30, 35, 36, 37, 38, 41C, 51, 63, 64, 138D, I, |
| | II, IV, NN-Bacillus (13), Bat10, BSL10, BSL11, BS6, |
| | BS11, BS16, BS23, BS101, BS102, g18, mor1, PBL1, |
| | SN45, thu2, thu3, Tm1, Tm2, TP-20, TP21, TP52, type |
| | F, type G, type IV, NN-Bacillus (3) BLE, (syn = θc), BS2, |
| | BS4, BS5, BS7, B10, B12, BS20, BS21, F, MJ-4, |
| | PBA12, AP50, AP50-04, AP50-11, AP50-23, AP50-26, |
| | AP50-27, Bam35 |
| Bacteroides | Bf-41, ad1₂, Baf-44, Baf-48B, Baf-64, Bf-1, Bf-52, B40-8, |
| | F1, β1, φA1, φBr01, φBr02, 67.1, 67.3, 68.1, NN- |
| | Bacteroides (3) |
| Bdellovibrio | MAC-1, MAC-1′, MAC-2, MAC-4, MAC-4′, MAC-5, MAC- |
| | 7, MAC-1, MAC-1′, MAC-2, MAC-4, HDC-2, MAC-6, |
| | VL-1 |
| Bacteroides | Bf42, Bf71, NN-Bdellovibrio (1) |
| Borrelia | NN-Borrelia (2), NN-Borrelia (1) |
| Brucella | A422, Bk, (syn = Berkeley), BM₂₉, FO₁, (syn = FO1), |
| | (syn = FQ1), D, FP₂, (syn = FP2), (syn = FD2), Fz, (syn = Fz75/ |
| | 13), (syn = Firenze 75/13), (syn = Fi), F₁, (syn = F1), |
| | F₁m, (syn = F1m), (syn = Fim), F₁U, (syn = F1U), (syn = FiU), |
| | F₂, (syn = F2), F₃, (syn = F3), F₄, (syn = F4), F₅, |
| | (syn = F5), F₆, F₇, (syn = F7), F₂₅, (syn = F25), (syn = f25), |
| | F₂₅U, (syn = F₂₅u), (syn = F25U), (syn = F25V), F₄₄, (syn = F44), |
| | F₄₅, (syn = F45), F₄₈, (syn = F48), I, Im, M, MC/75, |
| | M51, (syn = M85), P, (syn = D), S708, R, Tb, (syn = TB), |
| | syn = Tbilisi), W, (syn = Wb), (syn = Weybridge), X, |
| | 3, 6, 7, 10/1, (syn = 10), (syn = F₈), (syn = F8), 12m, 24/II, (syn = 24), |
| | (syn = F₉), (syn = F9), 45/III, (syn = 45), 75, 84, |
| | 212/XV, (syn = 212), (syn = F₁₀), (syn = F10), 371/XXIX, |
| | (syn = 371), syn = F₁₁), (syn = F11), 513 |
| Burkholderia | CP75, NN-Burkholderia (1) |
| Campylobacter | C type, NTCC12669, NTCC12670NTCC12671, |
| | NTCC12672, NTCC12673NTCC12674, NTCC12675, |
| | NTCC12676NTCC12677, NTCC12678, |
| | NTCC12679NTCC12680, NTCC12681, |
| | NTCC12682, NTCC12683, NTCC12684, 32f, 111c, |
| | 191NN-Campylobacter (2), Vfi-6, (syn = V19), Vfv-3V2, |
| | V3, V8, V16, (syn = Vfi-1), V19, V20(V45), V45, (syn = V- |
| | 45) NN-Campylobacter (1) |
| Caulobacter | φCr24, φCr26, φCr30, φCr35, φCb5, φCb8r φCb12r, |
| | φCb23r, φCp2, φCp14, φCr14, φCr28 φCd1, φCr40, φCr41, |
| | φCr1, φCr22, φ101, φ102φ118, φ151, φ6, 76, φCbK, |
| | φCb3, φCb6φCb13, φCp34, φCr2, φCr4, φCr5, φCr6, |
| | φCr7, φCr8, φCr9, φCr10, φCr11, φCr12, φCr13, φCr15, |
| | φCr20, φCr21, φCr23, φCr25, φCr27, φCr29, φCr31, |
| | φCr32, φCr33, φCr34, φCr36, φCr37, φCr38, φCr39, |
| | φCr42, φCr43 |
| Citrobacter | FC3-9, FC3-8 |
| Clostridium | F1, HM7, HM3, CEB, CA5, Ca7, CEβ, (syn = 1C), CEγ, |
| | Cld1, c-n71, c-203 Tox−, DEβ(syn = 1D), (syn = 1D^{tox+}), |
| | HM3, KM1, KT, Ms, NA1, (syn = Na1^{tox+}), PA1350e, Pfo, |
| | PL73, PL78, PL81, P1, P50, P5771, P19402, 1C^{tox+}, |
| | 2C^{tox-}, 2D, (syn = 2D^{tox+}), 3C, (syn = 3C^{tox+}), 4C, (syn = 4C^{tox+}), |
| | 56, III-1, NN-Clostridium (61)CAK1, CA1, HMT, |
| | HM2, PF1, P-₂₃, P-₄₆, Q-₀₅, Q-₀₆, Q-₁₆, Q-₂₁, Q-₂₆, Q-₄₀, |
| | Q-₄₆, S₁₁₁, SA₀₂WA₀₁, WA₀₃, W₁₁₁, W₅₂₃, 80, C, CA2, |
| | CA3, CPT1, CPT4, c1, c4, c5, HM7, H₁₁/A₁, H₁₈/A₁ |
| | H₂₂/S₂₃, H₁₅₈/A₁, K₂/A₁, K₂₁/S₂₃, M_{L}, NA2^{tox-}, Pf2, Pf3, |
| | Pf4, S₉/S₃, S₄₁/A₁, S₄₄/S₂₃, α2, 41, 112/S₂₃, 214/S₂₃, |
| | 233/A₁, 234/S₂₃, 235/S₂₃, II-1, II-2, II-3 CA1, F1, K, S2, |
| | 1, 5, NN-Clostridium (8) NN-Clostridium (12) |
| Coliform | AE2, dA, Ec9, f1, fd, HR, M13, ZG/2, ZJ/2 |
| Coryneforms | Arp, BL3, CONX, MT, Beta, A8010, A19, A A2, A3, A101, |
| | A128, A133, A137, A139, A155, A182, B, BF, B17, |
| | B18, B51, B271, B275, B276, B277, B279, B282, C, |
| | cap₁, CC1, CG1, CG2, CG33, CL31, Cog, (syn = CG5), D, |
| | E, F, H, H-1, hq₁, hq₂, I₁/H₃₃, I₁/31, J, K, K, (syn = K^{tox-}), |
| | L, L, (syn = L^{tox+}), M, MC-1, MC-2, MC-3, MC-4, |
| | MLMa, N, O, ov₁, ov₂, ov₃, P, P, R, RP6, Rₛ29, S, T, |
| | U, UB₁, ub₂, UH₁, UH₃, uh₃, uh₅, uh₆, β, (syn = β^{tox+}), |
| | β^{hv64}, βvir, γ, (syn = γ^{tox-}), γ19, δ, (syn = δ^{tox+}), ρ, (syn = ρ^{tox-}), |
| | φ9, φ984, ω, 1A, 1/1180, 2, 2/1180, 5/1180, |
| | 5ad/9717, 7/4465, 8/4465, 8ad/10269, 0/9253, 13/9253, |
| | 15/3148, 21/9253, 28, 29, 55, 2747, 2893, 4498, 5848. |
| | CGK1 |
| Cyanobacteria | S-2L, S-4L, N1, AS-1, S-6(L) |
| Enterobacter | C3, WS-EO20, WS-EP26, WS-EP28, φmp 667/617, 886 |
| | C-2, If1, If2, Ike, I2-2, PR64FS, SF, tf-1, PRD1, H-19J, B6, |
| | B7, C-1, C2, Jersey, ZG/3A, T5, ViII, WS-EP57, 379/319 |
| | b4, chi, Beccles, tu, PRR1, 7s, C-1, c2, fcan, folac, Ialpha, |
| | M, pilhalpha, R23, R34, ZG/1, ZIK/1, ZJ/1, ZL/3, ZS/3, |
| | alpha15, f2, fr, FC3-9, K19, Mu, 01, P2, ViI, \hat{ }92, 121, 16-19, |
| | 9266, C16, DdVI, PST, SMB, SMP2, a1, 3, 3T+, 9/0, |
| | 11F, 50, 66F, 5845, 8893, M11, QB, ST, TW18, VK, FI, |
| | ID2, fr, f2, AE2, Ec9, C-2 f1, (syn = f-1), HR, IF1, IF2, |
| | IKe, I₂-2, M13, (syn = M-13), PR64FS, SF, tf-1, X, X-2, |
| | ZG/2, ZJ2, δA B6, B7, C-1, C2, FH5, Fₒlac, fr, f2, |
| | (syn = f₂), Hgal, Iα, M, MS2, M12, (syn = M-12), pilHα, |
| | R17, (syn = R-17), SR, t, ZG/1, ZIK/1, ZJ/1, ZL/3, ZS/3, |
| | α15, μ2, (syn = μ₂) BE/1, dφ3, dφ4, dφ5, G4, G6, G13, |
| | G14, 1φ1, 1φ3, 1φ7, 1φ9, M20, St-1, (syn = St/1), (syn = ST-1), |
| | S13, (syn = S-13), U3, WA/1, WF/1, WW/1, |
| | ZD13, α3, α10, δ1, η8, o6, φA, φR, (syn = φX), (syn = φX-174), |
| | (syn = ΦX174), ζ3, WS-EP13, WS-EP19 |
| Enterococcus | DF₇₈, F1, F2, 1, 2, 4, 14, 41, 867, C2, C2F E3, E62, |
| | DS96, H24, M35, P3, P9, SB101, S2, 2BII, 5, 182a, |
| | 705, 873, 881, 940, 1051, 1057, 21096C, F3, F4, VD13, |
| | 1, 200, 235, 341 |
| Erwinia | E15P, PEa7, Y46/(CE2), PEal(h), S1, φM1, Erh1, E16P, |
| | 59, 62, 843/60 |
| Escherichia | BW73, B278, D6, D108, E, E1, E24, E41, FI-2, FI-4, |
| | FI-5, HI8A, HI8B, i, MM, Mu, (syn = mu), (syn = Mu1), |
| | (syn = Mu-1), (syn = MU-1), (syn = MuI), (syn = μ), O25, |
| | PhI-5, Pk, PSP3, P1, P1D, P2, P4 (defective), S1, Wφ, |
| | φK13, φR73 (defective), φ1, φ2, φ7, φ92, ψ (defective), |
| | 7A, 8φ, 9φ, 15 (defective), 18, 28-1, 186, 299, NN- |
| | Escherichia (2) CFO103, HK620, J, K, K1F, m59, no. A, no. |
| | E, no. 3, no. 9, N4, sd, (syn = Sd), (syn = S_{D}), (syn = S_{d}), |
| | (syn = S_{d}), (syn = SD), (syn = CD), T3, (syn = T-3), (syn = T₃), |
| | T7 (syn = T-7), (syn = T₇), WPK, W31, Δ^{H}, φC3888, φK3, |
| | φK7, φK12, φV-1, Φ04-CF, Φ05, Φ06, Φ07, φ1, φ1.2, |
| | φ20, φ95, φ263, φ1092, φI, φII, (syn = φW), Ω8, 1, 3, 7, 8, 26, |
| | 27, 28-2, 29, 30, 31, 32, 38, 39, 42, 933W NN- |
| | Escherichia (1), Esc-7-11, AC30, CVX-5, C1, DDUP, EC1, |
| | EC2, E21, E29, F1, F26S, F27S, Hi, HK022, HK97, |
| | (syn = ΦHK97), HK139, HK253, HK256, K7, ND-1, no. D, |
| | PA-2, q, S2, T1, (syn = α), (syn = P28), (syn = T-1), |
| | (syn = T₁), T3C, T5, (syn = T-5), (syn = T₅), UC-1, w, β4, |
| | γ2, λ, (syn = Φλ), ΦD326, φγ, Φ06, Φ7, Φ10, φ80, χ, |
| | (syn = χ₁), (syn = φχ), (syn = φχ₁), 2, 4, 4A, 6, 8A 102, |
| | 150, 168, 174, 3000, AC6, AC7, AC28, AC43, AC50, |
| | AC57, AC81, AC95, HK243, K10, ZG/3A, 5, 5A, 21EL, |
| | H19-J, 933H |
| Haemophilus | HP1, S2 |
| Klebsiella | AIO-2, Kl₄B, Kl₆B, Kl₉, (syn = Kl₉), Kl₁₄, Kl₁₅, Kl₂₁, |
| | Kl₂₈, Kl₂₉, Kl₃₂, Kl₃₃, Kl₃₅, Kl₁₀₆B, Kl₁₇₁B, Kl₁₈₁B, Kl₈₃₂B, |
| | CI-1, Kl₄B, Kl₈, Kl₁₁, Kl₁₂, Kl₁₃, Kl₁₆, Kl₁₇, Kl₁₈, Kl₂₀, |
| | Kl₂₂, Kl₂₃, Kl₂₄, Kl₂₆, Kl₃₀, Kl₃₄, Kl₁₀₆B, Kl₁₆₅B, Kl₃₂₈B, |
| | KLXI, K328, P5046, 11, 380, III, IV, VII, VIII, FC3-11, |
| | Kl₂B, (syn = Kl2B), Kl₂₅, (syn = Kl25), Kl₄₂B, (syn = Kl42), |
| | (syn = Kl42B), Kl₁₈₁B, (syn = Kl181), (syn = Kl181B), |
| | Kl_{765/1}, (syn = Kl765/1), Kl₈₄₂B, (syn = Kl832B), Kl₉₃₇B, |
| | (syn = Kl937B), L1, φ28, 7, 231, 483, 490, 632 Listeria |
| | H387, 2389, 2671, 2685, 4211, A511, O1761, 4211, 4286, |
| | (syn = BO54), A005, A006, A020, A500, A502, A511, |
| | A118, A620, A640, B012, B021, B024, B025, B035, |
| | B051, B053, B054, B055, B056, B101, B110, B545, |
| | B604, B653, C707, D441, HSO47, H1OG, H8/73, H19, |
| | H21, H43, H46, H107, H108, H110, H163/84, H312, |
| | H340, H387, H391/73, H684/74, H924A, PSA, U153, |
| | φMLUP5, (syn = P35), 00241, 00611, 02971A, 02971C, |
| | 5/476, 5/911, 5/939, 5/11302, 5/11605, 5/11704, 184, 575, |
| | 633, 699/694, 744, 900, 1090, 1317, 1444, 1652, 1806, 1807, |
| | 1921/959, 1921/11367, 1921/11500, 1921/11566, |
| | 1921/12460, 1921/12582, 1967, 2389, 2425, 2671, 2685, |
| | 3274, 3550, 3551, 3552, 4276, 4277, 4292, 4477, 5337, |
| | 5348/11363, 5348/11646, 5348/12430, 5348/12434, 10072, |
| | 11355C, 11711A, 12029, 12981, 13441, 90666, 90816, |
| | 93253, 907515, 910716, NN-Listeria (15) |
| Micrococcus | N1, N5 |
| Mycobacterium | Lacticola, Leo, R1-Myb, 13, AG1, AL₁, ATCC 11759, A2, |
| | B.C₃, BG2, BK1, BK₅, butyricum, B-1, B5, B7, B30, |
| | B35, Clark, C1, C2, DNAIII, DSP₁, D4, D29, GS4E, |
| | (syn = GS₄E), GS7, (syn = GS-7), (syn = GS₇), IPα, lacticola, |
| | Legendre, Leo, L5, (syn = ΦL-5), MC-1, MC-3, MC-4, |
| | minetti, MTPH11, Mx4, MyF₃P/59a, phlei, (syn = phlei 1), |
| | phlei 4, Polonus II, rabinovitschi, smegmatis, TM4, TM9, |
| | TM10, TM20, Y7, Y10, φ630, 1B, 1F, 1H, 1/1, 67, 106, |
| | 1430, B1, (syn = Bo1), B₂₄, D, D29, F-K, F-S, HP, |
| | Polonus I, Roy, R1, (syn = R1-Myb), (syn = R₁), 11, 31, |
| | 40, 50, 103a, 103b 128, 3111-D, 3215-D, NN- |
| | Mycobacterium (1) |
| Mycoplasma | MV-G51, NN-Mycoplasma (1), Hr1, P1 |
| Pasteurella | C-2, 32, AU, VL, TX, φPhA1, 1, 2, 10, 3/10, 4/10, |
| | 115/10, 895, 3, 22, 55, 115, 896, 994, 995, B932a, C-2, |
| | φPhA1, 32, 53, 115, 967, 1075 |
| Proteus | Pm5, 13vir, 2/44, 4/545, 6/1004, 13/807, 20/826, 57, 67b, |
| | 78, 107/69, 121, Pm1, Pm3, Pm4, Pm6, Pm7, Pm9, |
| | Pm10, Pm11, Pv2, π1, φm, 7/549, 9B/2, 10A/31, 12/55, |
| | 14, 15, 16/789, 17/971, 19A/653, 23/532, 25/909, 26/219, |
| | 27/953, 32A/909, 33/971, 34/13, 65, 5006M, 7480b, VI, |
| | 13/3a Clichy 12, π2600, φχ7, 1/1004, 5/742, 9, 12, 4, 22, |
| | 24/860, 2600/D52, Pm8, 24/25 14 |
| Pseudomonas | Phi6, Pf1, Pf2, Pf3, D3, Kf1, M6, PS4, SD1, PB-1, PP8, |
| | PS17, nKZ, nW-14, n1, 12S, AI-1, AI-2, B17, B89, CB3, |
| | Col 2, Col 11, Col 18, Col 21, C154, C163, C167, C2121, |
| | E79, F8, ga, gb, H22, K₁, M4, N₂, Nu, PB-1, (syn = PB1), |
| | pf16, PMN17, PP1, PP8, Psa1, PsP1, PsP2, PsP3, |
| | PsP4, PsP5, PS3, PS17, PTB80, PX4, PX7, PYO1, |
| | PYO2, PYO5, PYO6, PYO9, PYO10, PYO13, PYO14, |
| | PYO16, YO18, PYO19, PYO20, PYO29, PYO32, PYO33, |
| | PYO35, PYO36, PYO37, PYO38, PYO39, PYO41, |
| | PYO42, PYO45, PYO47, PYO48, PYO64, PYO69, |
| | PYO103, P1K, SLP1, SL2, S₂, UNL-1, wy, Ya₁, Ya₄, |
| | Ya₁₁, φBE, φCTX, φC17, φKZ, (syn = φKZ), φ-LT, |
| | Φmu78, φNZ, φPLS-1, φST-1, φW-14, φ-2, 1/72, 2/79, |
| | 33/DO, 4/237, 5/406, 6C, 6/6660, 7, 7v, 7/184, 8/280, |
| | 9/95, 10/502, 11/DE, 12/100, 12S, 16, 21, 24, 25F, 27, 31, |
| | 44, 68, 71, 95, 109, 188, 337, 352, 1214, NN- |
| | Pseudomonas (23), φ6, PP7, PRR1, 7s, NN-Pseudomonas |
| | (1), A856, B26, CI-1, CI-2, C5, D, gh-1, F116, HF, H90, |
| | K₅, K₆, K104, K109, K166, K267, N₄, N₅, O6N-25P, |
| | PE69, Pf, PPN25, PPN35, PPN89, PPN91, PP2, PP3, |
| | PP4, PP6, PP7, PP8, PP56, PP87, PP114, PP206, PP207, |
| | PP306, PP651, Psp231a, Pssy401, Pssy9220, ps₁, PTB2, |
| | PTB20, PTB42, PX1, PX3, PX10, PX12, PX14, PYO70, |
| | PYO71, R, SH6, SH133, tf, Ya₅, Ya₇, φBS, ΦKf77, φ- |
| | MC, ΦmnF82, φPLS27, φPLS743, φS-1, 1, 2, 2, 3, 4, 5, |
| | 6, 7, 7, 8, 9, 10, 11, 12, 12B, 13, 14, 15, 14, 15, 16, |
| | 17, 18, 19, 20, 20, 21, 21, 22, 23, 23, 24, 25, 31, 53, |
| | 73, 119x, 145, 147, 170, 267, 284, 308, 525, NN- |
| | Pseudomonas (5), af, A7, B3, B33, B39, BI-1, C22, D3, |
| | D37, D40, D62, D3112, F7, F10, g, gd, ge, gf, Hw12, |
| | Jb19, KF1, L°, OXN-32P, O6N-52P, PCH-1, PC13-1, |
| | PC35-1, PH2, PH51, PH93, PH132, PMW, PM13, |
| | PM57, PM61, PM62, PM63, PM69, PM105, PM113, |
| | PM681, PM682, PO4, PP1, PP4, PP5, PP64, PP65, |
| | PP66, PP71, PP86, PP88, PP92, PP401, PP711, PP891, |
| | Pssy41, Pssy42, Pssy403, Pssy404, Pssy420, Pssy923, |
| | PS4, PS-10, Pz, SD1, SL1, SL3, SL5, SM, φC5, φC11, |
| | φC11-1, φC13, φC15, φMO, φX, φ04, φ11, φ240, 2, 2F, |
| | 5, 7m, 11, 13, 13/441, 14, 20, 24, 40, 45, 49, 61, 73, |
| | 148, 160, 198, 218, 222, 236, 242, 246, 249, 258, 269, |
| | 295, 297, 309, 318, 342, 350, 351, 357-1, 400-1, NN- |
| | Pseudomonas (6), G101, M6, M6a, L1, PB2, Pssy15, |
| | Pssy4210, Pssy4220, PYO12, PYO34, PYO49, PYO50, |
| | PYO51, PYO52, PYO53, PYO57, PYO59, PYO200, |
| | PX2, PX5, SL4, φ03, φ06, 1214 |
| Salmonella | B, Beccles, CT, d, Dundee, f, Fels 2, GI, GIII, GVI, |
| | GVIII, k, K, i, j, L, O1, (syn = O-1), (syn = O₁), (syn = O- |
| | I), (syn = 7), O2, O3, P3, P9a, P10, Sab3, Sab5, San15, |
| | San17, SI, Taunton, Vi1, (syn = ViI), 9, NN-Salmonella |
| | (1), a, B.A.O.R., e, G4, GIII, L, LP7, M, MG40, N-18, |
| | PSA68, P4, P9c, P22, (syn = P₂₂), (syn = PLT22), (syn = PLT₂₂), |
| | P22a1, P22-4, P22-7, P22-11, SNT-1, SNT-2, |
| | SP6, ViIII, ViIV, ViV, ViVI, ViVII, Worksop, ε₁₅, ε₃₄, |
| | 1, 37, 1(40), (syn = φ1[40]), 1, 42₂, 2, 2.5, 3b, 4, 5, |
| | 6, 14(18), 8, 14(6, 7), 10, 27, 28B, 30, 31, 32, 33, 34, |
| | 36, 37, 39, 1412, SNT-3, 7-11, 40.3, c, C236, C557, |
| | C625, C966N, g, GV, G5, G173, h, IRA, Jersey, MB78, |
| | P22-1, P22-3, P22-12, Sab1, Sab2, Sab2, Sab4, San1, |
| | San2, San3, San4, San6, San7, San8, San9, San13, |
| | San14, San16, San18, San19, San20, San21, San22, |
| | San23, San24, San25, San26, SasL1, SasL2, SasL3, |
| | SasL4, SasL5, S1BL, SII, ViII, φ1, 1, 2, 3a, 3aI, 1010, |
| | NN-Salmonella (1), N-4, SasL6, 27 |
| Serratio | A2P, PS20, SMB3, SMP, SMP5, SM2, V40, V56, κ, |
| | ΦCP-3, ΦCP-6, 3M, 10/1a, 20A, 34CC, 34H, 38T, |
| | 345G, 345P, 501B, E20, P8, Sa1, SM4, η, ΦCP6-4, 5E, |
| | 34D, 38B, 224D1, 224D2, 2847/10b, BC, BT, CW2, |
| | CW3, CW4, CW5, L₁232, L₂232, L34, L.228, SLP, |
| | SMPA, V.43, σ, φCW1, ΦCP6-1, ΦCP6-2, ΦCP6-5, 3T, |
| | 5, 8, 9F, 10/1, 20E, 32/6, 34B, 34CT, 34P, 37, 41, 56, |
| | 56D, 56P, 60P, 61/6, 74/6, 76/4, 101/8900, 226, 227, |
| | 228, 229F, 286, 289, 290F, 512, 764a, 2847/10, |
| | 2847/10a, L.359, SMB1 |
| Shigella | Fsa, a, FS_{D2d}, (syn = D2d), (syn = W₂d), FS_{D2E}, (syn = W₂e), |
| | Fv, F6, f7.8, H-Sh, PE5, P90, SfII, Sh, SH_{III}, SH_{IV}, |
| | (syn = HIV), SH_{VI}, (syn = HVI), SHV_{VIII}, (syn = HVIII), |
| | SKγ66, (syn = gamma 66), (syn = γ66), (syn = γ66b), SK_{III}, |
| | (syn = SIIIb), (syn = III), SK_{IV}, (syn = S_{IVa}), (syn = IV), |
| | SK_{IVa}, (syn = S_{IVAa}), (syn = IVA), SK_{VI}, (syn = KVI), (syn = S_{VI}), |
| | (syn = VI), SK_{VIII}, (syn = S_{VIII}), (syn = VIII), SK_{VIIIA}, |
| | (syn = S_{VIII}A), (syn = VIIIA), ST_{VI}, ST_{IX}, ST_{XI}, ST_{XII}, S66, |
| | W₂, (syn = D2c), (syn = D20), φI, fIV₁, 3-SO-R, 8368- |
| | SO-R, DD-2, Sf6, FS₁, (syn = F1), SF₆, (syn = F6), SG₄₂, |
| | (syn = SO-42/G), SG₃₂₀₃, (syn = SO-3203/G), SK_{F12}, (syn = SsF₁₂), |
| | (syn = F₁₂), (syn = F12), ST_{II}, (syn = 1881-SO-R), |
| | γ66, (syn = gamma 66a), (syn = Ssγ66), φ2 B11, DDVII, |
| | (syn = DD7), FS_{D2b}, (syn = W₂B), FS₂, (syn = F₂), (syn = F2), |
| | FS₄, (syn = F₄), (syn = F4), FS₅, (syn = F₅), (syn = F5), FS₉, |
| | (syn = F₉), (syn = F9), F11, P2-SO-S, SG₃₆, (syn = SO- |
| | 36/G), (syn = G36), SG₃₂₀₄, (syn = SO-3204/G), SG₃₂₄₄, |
| | (syn = SO-3244/G), SH₁, (syn = HI), SH_{VII}, (syn = HVII), |
| | SH_{IX}, (syn = HIX), SH_{XI}, SH_{XII}, (syn = HXII), SKI, KI, |
| | (syn = S_{I}), (syn = SsI), SKVII, (syn = KVII), (syn = S_{VII}), |
| | (syn = SsVII), SKIX, (syn = KIX), (syn = S_{IX}), (syn = SsIX), |
| | SKXII, (syn = KXII), (syn = S_{XII}), (syn = SsXII), ST_{I}, ST_{III}, |
| | ST_{IV}, ST_{VI}, ST_{VII}, S70, S206, U2-SO-S, 3210-SO-S, |
| | 3859-SO-S, 4020-SO-S, φ3, φ5, φ7, φ8, φ9, φ10, φ11, |
| | φ13, φ14, φ18, SH_{III}, (syn = HIII), SH_{XI}, (syn = HXI), |
| | SK_{XI}, (syn = KXI), (syn = S_{XI}), (syn = SsXI), (syn = XI) |
| Staphylococcus | 3A, B11-M15, 77, 107, 187, 2848A, Twort A, EW, K, Ph5, |
| | Ph9, Ph10, Ph13, P1, P2, P3, P4, P8, P9, P10, RG, |
| | S_{B-1}, (syn = Sb-1), S3K, φSK311, φ812, 06, 40, 58, 119, |
| | 130, 131, 200, 1623, STC1, (syn = stc1), STC2, (syn = stc2), |
| | 44AHJD, 68, AC1, AC2, A6“C”, A9“C”, b⁵⁸¹, CA-1, |
| | CA-2, CA-3, CA-4, CA-5, D11, L39 × 35, L54a, M42, |
| | N1, N2, N3, N4, N5, N7, N8, N10, N11, N12, N13, |
| | N14, N16, Ph6, Ph12, Ph14, UC-18, U4, U15, S1, S2, |
| | S3, S4, S5, X2, Zₜ, φB5-2, φD, ω, 11, (syn = φ11), |
| | (Syn = P11-M15), 15, 28, 28A, 29, 31, 31B, 37, 42D, |
| | (syn = P42D), 44A, 48, 51, 52, 52A, (syn = P52A), 52B, |
| | 53, 55, 69, 71, (syn = P71), 71A, 72, 75, 76 77, 79, 80, |
| | 80α, 82, 82A, 83A, 84, 85, 86, 88, 88A, 89, 90, 92, 95, |
| | 96, 102, 107, 108, 111, 129-26, 130, 130A, 155, 157, |
| | 157A, 165, 187, 275, 275A, 275B, 356, 456, 459, 471, |
| | 471A, 489, 581, 676, 898, 1139, 1154A, 1259, 1314, |
| | 1380, 1405, 1563, 2148, 2638A, 2638B, 2638C, 2731, |
| | 2792A, 2792B, 2818, 2835, 2848A, 3619, 5841, 12100, |
| | AC3, A8, A10, A13, b594n, D, HK2, N9, N15, P52, |
| | P87, S1, S6, Z₄, φRE, 3A, 3B, 3C, 6, 7, 16, 21, 42B, |
| | 42C, 42E, 44, 47, 47A, 47C, 51, 54, 54x1, 70, 73, 75, |
| | 78, 81, 82, 88, 93, 94, 101, 105, 110, 115, 129/16, 174, |
| | 594n, 1363/14, 2460, NN-Staphylococcus (1) |
| Streptococcus | A25, A25 PE1, A25 VD13, A25 omega8, EJ-1, NN- |
| | Streptococcus (1), a, C1, F_{LO}Ths, H39, Cp-1, Cp-5, Cp-7, |
| | Cp-9, Cp-10, AT298, A5, a10/J1, a10/J2, a10/J5, |
| | a10/J9, A25, BT11, b6, CA1, c20-1, c20-2, DP-1, Dp-4, |
| | DT1, ET42, e10, F_{A}101, F_{E}Ths, F_{K}, F_{KK}101, F_{KL}10, |
| | F_{KP}74, F_{K}11, F_{LO}Ths, F_{Y}101, f1, F₁₀, F₂₀140/76, g, GT- |
| | 234, HB3, (syn = HB-3), HB-623, HB-746, M102, O1205, |
| | φO1205, PST, P0, P1, P2, P3, P5, P6, P8, P9, P9, |
| | P12, P13, P14, P49, P50, P51, P52, P53, P54, P55, P56, |
| | P57, P58, P59, P64, P67, P69, P71, P73, P75, P76, |
| | P77, P82, P83, P88, sc, sch, sf, Sfi11, (syn = SFi11), |
| | (syn = φSFi11), (syn = ΦSfi11), (syn = φSfi11), sfi19, (syn = SFi19), |
| | (syn = φSFi19), (syn = φSfi19), Sfi21, (syn = SFi21), |
| | (syn = φSFi21), (syn = φSfi21), ST_{G}, STX, st2, ST₂, ST₄, |
| | S3, (syn = φS3), s265, Φ17, φ42, Φ57, φ80, φ81, φ82, |
| | φ83, φ84, φ85, φ86, φ87, φ88, φ89, φ90, φ91, φ92, φ93, |
| | φ94, φ95, φ96, φ97, φ98, φ99, φ100, φ101, φ102, φ227, |
| | Φ7201, ω1, ω2, ω3, ω4, ω5, ω6, ω8, ω10, 1, 6, 9, |
| | 10F, 12/12, 14, 17SR, 19S, 24, 50/33, 50/34, 55/14, |
| | 55/15, 70/35, 70/36, 71/ST15, 71/45, 71/46, 74F, 79/37, |
| | 79/38, 80/J4, 80/J9, 80/ST16, 80/15, 80/47, 80/48, 101, |
| | 103/39, 103/40, 121/41, 121/42, 123/43, 123/44, 124/44, |
| | 337/ST17, NN-Streptococcus (34) |
| Streptomyces | SK1, type IV, CRK, SLE111, Φ17, (syn = φ17), (syn = 2a), |
| | 1, 9, 14, 24 A, AP-3, AP-2863, Bα, B-I, B-II, CPC, |
| | CPT, CT, CTK, CWK, ES, FP22, FP43, K, MSP4, |
| | MSP7, MSP10, MSP11, MSP15, MSP16, MSP17, |
| | MSP18, MSP19, MVP4, MVP5, P8, P9, P13, P23, |
| | RP2, RP3, RP10, R₁, R4, SAP1, SAP2, SAP3, SAt1, |
| | SA6, SA7, SC1, SH10, SL1, SV2, TG1, type Ia, type II, |
| | type V, VC11, VP1, VP5, VP7, VP11, VWB, VW3, |
| | WSP3, φA1, φA2, φA3, φA4, φA5, φA6, φA7, φA8, |
| | φA9, φBP1, φBP2, φC31, (syn = φc31), (syn = φ31C), |
| | (syn = C31), φC43, φHAU3, φSF1, φSPK1, 4, 5a, 5b, 8, |
| | 10, 12b, 13, 17, 19, 22, 23, 25, 506, NN-Streptomyces |
| | (3), Mex, MLSP1, MLSP2, MLSP3, MSP1, MSP2, |
| | MSP3, MSP5, MSP8, MSP9, MSP12, MSP13, MSP14, |
| | R₂, SA1, SA2, SA3, SA4, SA5, type I, type Ia., (syn = 35/35), |
| | type III, type IV, WSP1, WSP4, WSP5, 2b, 4, |
| | 15, (syn = C), 26, 8238 |
| Vibrio | OXN-52P, VP-3, VP5, VP11, alpha3alpha, IV, kappa, 06N- |
| | 22-P, VP1, x29, II, nt-1, CP-T1, ET25, kappa, K139, |
| | Labol, OXN-69P, OXN-86, O6N-21P, PB-1, P147, rp-1, |
| | SE3, VA-1, (syn = VcA-1), VcA-2, VcA-3, VP1, VP2, |
| | VP4, VP7, VP8, VP9, VP10, VP17, VP18, VP19, X29, |
| | (syn = 29 d'Hérelle), β, ΦHAWI-1, ΦHAWI-2, ΦHAWI-3, |
| | ΦHAWI-4, ΦHAWI-5, ΦHAWI-6, ΦHAWI-7, |
| | ΦHAWI-8, ΦHAWI-9, ΦHAWI-10, ΦHC1-1, ΦHC1-2, |
| | ΦHC1-3, ΦHC1-4, ΦHC2-1, ΦHC2-2, ΦHC2-3, ΦHC2- |
| | 4, ΦHC3-1, ΦHC3-2, ΦHC3-3, ΦHD1S-1, ΦHD1S-2, |
| | ΦHD2S-1, ΦHD2S-2, ΦHD2S-3, ΦHD2S-4, ΦHD2S-5, |
| | ΦHDO-1, ΦHDO-2, ΦHDO-3, ΦHDO-4, ΦHDO-5, |
| | ΦHDO-6, KL-33, ΦKL-34, ΦKL-35, ΦKL-36, ΦKWH- |
| | 2, ΦKWH-3, ΦKWH-4, ΦMARQ-1, ΦMARQ-2, |
| | ΦMARQ-3, ΦMOAT-1, ΦO139, ΦPEL1A-1, |
| | ΦPEL1A-2, ΦPEL8A-1, ΦPEL8A-2, ΦPEL8A-3, |
| | ΦPEL8C-1, ΦPEL8C-2, ΦPEL13A-1, ΦPEL13B-1, |
| | ΦPEL13B-2, ΦPEL13B-3, ΦPEL13B-4, ΦPEL13B-5, |
| | ΦPEL13B-6, ΦPEL13B-7, ΦPEL13B-8, ΦPEL13B-9, |
| | ΦPEL13B-10, φVP143, φVP253, Φ16, φ138, 1-11, 5, 13, |
| | 14, 16, 24, 32 493, 6214, 7050, 7227, II, (syn = group II), |
| | (syn = φ2), V, VIII, NN-Vibrio (13), CTXΦ, fs, (syn = fs1), |
| | fs2, lvpf5, Vf12, Vf33, VPIΦ, VSK, v6, 493, e1, |
| | e2, e3, e4, e5, FK, G, J, K, nt-6, N1, N2, N3, N4, |
| | N5, O6N-34P, OXN-72P, OXN-85P, OXN-100P, P, Ph-1, |
| | PL163/10, Q, S, T, φ92, 1-9, 37, 51, 57, 70A-8, 72A- |
| | 4, 72A-10, 110A-4, 333, 4996, I, (syn = group I), III, |
| | (syn = group III), VI, (syn = A-Saratov), VII, IX, X, NN- |
| | Vibrio (6), pA1, 77-8, 70A-2, 71A-6, 72A-5, 72A-8, |
| | 108A-10, 109A-6, 109A-8, 110A-1, 110A-5, 110A-7, hv- |
| | 1, OXN-52P, P13, P38, P53, P65, P108, P111, TP1, |
| | VP3, VP6, VP12, VP13, 70A-3, 70A-4, 70A-10, 72A-1, |
| | 108A-3, 109-B1, 110A-2, 149, (syn = φ149), IV, (syn = group |
| | IV), NN-Vibrio (22), VP5, VP11, VP15, VP16, α1, |
| | α2, α3a, α3b, 353B, NN-Vibrio (7) |
| Xanthomonas | Cf, Cflt, Xf, Xf2, XP5, HP1, OX1, (syn = XO1), OX2, |
| | SBX-1, XCVP₁, XTP1, Cf, Cflt, Xf, (syn = xf) Xf2, φLf, |
| | φXo, φXv, RR68, A342, HXX, PG60, P1-3a, P6, XO3, |
| | XO4, XO5, 20, 22, NN-Xanthomonas (1), XP12, (syn = XP- |
| | 12), (syn = Xp12), φPS, φRS, φSD, φSL, φ56, φ112, 1 |
| Yersinia | H, H-1, H-2, H-3, H-4, Lucas 110, Lucas 404, Lucas 303, |
| | YerA3, YerA7, YerA20, YerA41, 3/M64-76, 5/G394-76, |
| | 6/C753-76, 8/C239-76, 9/F18167, 1701, 1710, |
| | D'Hérelle, EV, H, Kotljarova, PTB, R, Y, YerA41, |
| | φYerO3-12, 3, 4/C1324-76, 7/F783-76, 903, 1/M6176, |
| | Yer2AT, |

(Phage names courtesy of Hans-Wolfgang Ackermann & Stephen Tobias Abedon (2001) Bacteriophage Names, 2000. The Bacteriophage Ecology Group, www.phage.org/names.htm)

There are numerous other phages infecting these and other bacteria. The bacteriophages are normally grouped into family, genus and species, including but not limited to the following genera. Bdellomicrovirus, Spiromcrovirus, Microvirus, Microvirus, Levivirus, Allolevivirus. It should be noted that the compositions of embodiments of the disclosure contain phage peptides and peptide fragments thereof as well as, or instead of, phage proteins.

These lytic proteins can be useful for the detection or treatment of the bacteria for which they are specific.

Prophylactic Methods

In one embodiment, the disclosure provides a method for preventing in a subject, a disease or condition associated with an aberrant expression or activity of a protein or protein peptide fragments of the disclosure, by administering to the subject an agent which modulates expression or at least one activity of the protein or protein peptide fragments. Administration of a prophylactic agent can occur prior to the manifestation of symptoms characteristic of the aberrancy, such that a disease or disorder is prevented or, alternatively, delayed in its progression. Depending on the type of aberrancy, for example, an agonist or antagonist agent can be used for treating the subject. The appropriate agent can be determined based on screening assays described herein.

Pharmaceutical Usage of Phage Associated Lytic Proteins

The method for prophylactically or therapeutically treating bacterial infections comprises treating the infection with a therapeutic agent comprising an effective amount of at least one lytic enzyme genetically coded for a bacteriophage specific for digesting the cell wall of a specific bacteria. The lytic enzyme is preferably in an environment having a pH which allows for activity of said lytic enzyme. The lytic enzyme may be “unaltered,” chimeric, shuffled or any combination thereof Additionally, a holin protein may be included in a composition containing the lytic enzyme(s).

The lytic enzyme can be used for the treatment or prevention of Streptococcus fasciae, Actinomyces (Actinomyces israelii), Agrobacterium, Alcaligenes, Bacillus (Bacillus anthracis), Bacteroides (Bacteroides fragilis), Bartonella (Bartonella bacilliformis, Bartonella henselae), Bdellovibrio, Bordetella (Bordetella pertussis), Borrelia (Borrelia burgdorferi, Borrelia recurrentis) Brucella (Brucella abortus, Brucella melitensis, Brucella suis) Burkholderia, Calymmatobacterium (Calymmatobacterium donovani), Campylobacter (Campylobacter fetus, Campylobacter jejuni), Caulobacter, Clostridium (Clostridium botulinum, Clostiridium difficile, Clostridium perfringens, Clostridium septicum, Clostridium tetani) Corynebacteria (Corynebacterium diptheriae), Coryneforms, Cyanobacteria, Enterobacteria (Enterobacter (Aerobacter) aerogenes), Escherschia coli, Francisella (Francisella tularensis), Haemophilus (Haemophilus ducreyi, Haemophilus influenzae), Klebsiella (Klebsiella ozaenae, Klebsiella pneumoniae, Klebsiella rhinoscleromatis), Lactobacillus, Lactoctococcus, Legionella (Legionella pneumophila), Leptospira, Listeria (Listeria monocytogenes), Micrococcus, Mollicutes, Mycobacteria (Mycobacterium avium, Mycobacterium, Mycobacterium intracellulare, Mycobacterium kansasii, Mycobacterium leprae, Mycobacterium tuberculosis, Mycobacterium ulcerans), Myxococcus, Neisseria (Neisseria gonorrhoeae, Neisseria meningitidis) Pasteurella, Pneumococci, Proteus (Proteus mirabilis, Proteus morgagni), Pseudomonas (Pseudomonas aeruginosa, Pseudomonas mallei, Pseudomonas pseudomalli), Rhizobium, Salmonella (Salmonella typhi, Salmonella typhimurium), Serratia (Serratia marcescens), Shigella, Spirillum (Spirillum minus), Spirochete (Spiroplasma), Staphylococci (Staphylococcus aureus), Streptobacillus (Streptobacillus moniliformis), Streptococci (Streptococcus pyogenes, Streptococcus pneumoniae), Treponema (Treponema carateum, Treponema pallidum, Treponema pertenue), Vibrio (Vibrio cholerae), Xanthomonas, Yersinia (Yersinia enterocolitica, Yersinia pestis, other bacteria, and any combination thereof. These illnesses and infections can be treated in both humans, animals, fowl, fish, etc. Various infections in various systems and parts of the body may be treated by the following compositions and methods.

Respiratory Infections

Most bacteria that are inhaled each day are either trapped in the nasopharnyx or eventually eliminated in mucus. Many bacteria (both pathogens and normal flora) remain in residue on the mucosal lining in the nasopharnyx. These represent the normal carrier state that resides inmost people. When disturbances occur in the immune system, this can result in a breakdown of normal system controls (homeostasis), which can often result in infection. Thus, elimination of the carrier state by enzymes can lead to a reduction in disease (i.e., prophylactic prevention by pre-treatment) of targeted enzymes. Those bacteria that manage to reach the alveoli of die lung are typically attacked by alveolar macrophages or by neutrophils. However, there are occasions when, for one reason or another, the system fails and an infection of the respiratory tract occurs. There are many reasons for this failure including, but not limited to, autoimmune disease, damage due to smoking or smoke inhalation, trauma, temporarily impaired immune system, vomiting, or numerous other causes. If there is a bacterial infection of the upper respiratory tract, or if the upper respiratory tract has possibly been exposed to a bacterial agent, the infection can be treated with a composition comprising: a) an effective amount of at least one lytic enzyme genetically coded for by a bacteriophage specific for the suspect bacteria and b) a means for delivering the lytic enzyme to a mouth, throat, or nasal passage. It is preferred that the lytic enzyme is in an environment having a pH which allows for activity of the lytic enzyme.

Additionally, if an individual has been exposed to someone with the upper respiratory disorder, this composition may be used to deliver the composition so that lytic enzyme will reside in the mucosal lining and prevent any colonization of the infecting bacteria.

The lytic enzyme is preferably a chimeric and/or shuffled lytic enzyme that may be used in conjunction with a holin enzyme or modified or unmodified phage associated lytic enzyme.

Two examples of bacteria which infect the upper respiratory system and which can be readily treated by the proposed therapeutic composition are Streptococcus pneumoniae and Hemophilus influenzae.

H. influenza is a gram negative bacteria. Colonizing in the pharynx, the encapsulated form of the bacteria can cause a lower respiratory tract infection. The unencapsulated form can cover the surface of the upper respiratory tract. Otitis of the middle ear may result, as can sinusitis, and bronchial pneumoniae. In the worst case scenarios, meningitis can develop, particularly in children.

In recent years, there has been an increase in the number of people, particularly children and the elderly, that are infected with or are carriers of penicillin resistant Hemophilus. While these bacteria are normally harmless residents of the host, they are opportunistic organisms that are able to cause infections when the resistance of the host has been compromised. By eliminating or reducing the number of these organisms in the upper respiratory tract, there will be a commensurate reduction in the number of infections by these bacteria.

Exposure of the Hemophilus bacteria to, for example, the lytic enzyme encoded for by the Bacteriophage HP1 (a member of the P2-like phage family with strong similarities to coliphages P2 and 186, and some similarity to the retronphage Ec67) lyses the cell wall of the bacteria.

Pneumococci account for the majority of cases of acute otitis media (AOM), community acquired pneumoniae, and bacterial meningitis, and can cause lethal sepsis. In recent years, resistance of pneumococci to multiple antibiotics has increased worldwide. Many studies have shown that treatment with antibiotics in children, be it for AOM or eradication of group A streptococci, even with a single dose, is associated with an increase in the carriage of resistant pneumococcal strains (E. Melander, et al., Eur J Clin Microbiol Infect Dis 17, 834-8. (1998), T. Heikkinen, et al., Acta Paediatr 89, 1316-21. (2000), and J. Y. Morita, et al., Paediatr Infect Dis J 19, 41-6. (2000), all incorporated by reference). Treatment of pneumococcal disease is thus becoming more difficult than in the past. The number of annual cases of AOM in the United States is about 7 million, while invasive pneumococcal infection was recently estimated to be more than 60,000 with an overall mortality of 10%. Although most of these latter cases occurred in persons eligible for vaccination (K. A. Robinson, et al., JAMA 285, 1729-35. (2001), incorporated by reference), vaccination rates remain insufficient (C. G. Stevenson, M. A. McArthur, M. Naus, E. Abraham, A. J. McGeer, CMAJ 164, 1413-9. (2001), S. Gleich, et al., Infect Control Hosp Epidemiol 21, 711-7. (2000) incorporated by reference). Furthermore, despite the progress that has been made with the development of conjugate vaccines for children younger than 2 years, it remains doubtful that vaccination alone is sufficient to eliminate carriage of and disease caused by pneumococci. The new conjugate vaccines include a restricted number of pneumococcal serotypes and protect only incompletely against colonization with these same strains. About one third to one half of cases of AOM are caused by strains not included in a 9-valent vaccine (S. I. Pelton, Vaccine 19 Suppl 1, S96-9. (2000), incorporated by reference). Moreover, an increase in the carriage of non-vaccine serotypes has been reported (N. Mbelle, et al., J Infect Dis 180, 1171-6. (1999), incorporated by reference). Because of these problems, there is a need for an alternative preventive strategy for situations where vaccination is insufficient, impossible or inefficient.

Eradication or even reduction of nasopharyngeal carriage likely will impact on the transmission of S. pneumoniae and the incidence of infection. Antibiotic prophylaxis in controlled surroundings has shown limited success but carries the risk of selective pressure resulting in an increase of resistant strains (S. D. Putnam, G. C. Gray, D. J. Biedenbach, R. N. Jones, Clin Microbiol Infect 6, 2-8. (2000). incorporated by reference).

A lytic enzyme specific for lysing the cell wall of Streptococcus pneumoniae is produced from genetic material from a bacteriophage specific for Streptococcus pneumoniae. This lytic enzyme may be administered to an individual with a Streptococcus pneumoniae infection, or to someone who has possibly been exposed to someone who does already have the infection. The lytic enzyme can be produced in a number of ways. In a preferred embodiment, a gene for the lytic enzyme from the phage genome is put into a transfer or movable vector, preferably a plasmid, and the plasmid is cloned into an expression vector or expression system. The expression vector may be E. coli, Bacillus, or a number of other suitable bacteria. The vector system may also be a cell free expression system. All of these methods of expressing a gene or set of genes are known in the art. The lytic enzyme may also be created by infecting Streptococcus pneumoniae with a bacteriophage specific for Streptococcus pneumoniae, wherein at least one lytic enzyme exclusively lyses the cell wall of said Streptococcus pneumoniae having at most minimal effects on other bacterial flora present.

The lytic enzyme for Streptococcus pneumoniae, previously identified as a -acetyl-muramoyl-L-alanine amidase, is genetically coded by the Pal bacteriophage, and can be produced by normal fermentation or recombinant and engineered processes. There are a number of bacteriophages for S. pneumoniae, including but not limited to Dp-1, DP4, Cp-1, Cp-7; Cp-9, Cp-5, MM1, EJ-1, HB-3, HB-623, HB-746, W-1, and W-2.

It is to be noted that all lytic enzymes produced for all of genus and species of bacteria can be made by these methods, using the genetic material of phages specific for producing a lytic enzyme that can specifically lyse the target bacteria.

Another infection which can be treated prophylactically is Streptococcus group A, which can produce what is commonly known as “strep” throat. When group C Streptococci are infected with a C1 bacteriophage, a lysin enzyme is produced specific for the lysing of Streptococcus group A. It should be noted that, as with all bacteria, the C1 bacteriophage is not the only phage to code for an enzyme specific for the destruction of the cell wall for Streptococcus group A.

There are many other bacteria which can be treated, using phage associated lytic enzymes. Bacillus anthracis, while not normally found in developed or even most developing nations, has regrettably been cited as a possible bio terror weapon. Indeed, the dormant and durable spore form of Bacillus anthracis is an ideal biological weapon of mass destruction. (Mock, M. & Fouet, A. Anthrax. Annu Rev Microbiol 55, 647-71 (2001), Inglesby, T. V. et al. “Anthrax as a Biological Weapon, 2002: Updated Recommendations for Management.” Jama 287, 2236-52 (2002)). Bacillus anthracis spores are extremely virulent in humans and can remain viable in the environment almost indefinitely after release. Once inhaled, spores are transported by alveolar macrophages to mediastinal and peribronchial lymph nodes where they germinate; subsequent vegetative clonal expansion causes an overwhelming bacteremia and toxemia. Mortality rates associated with untreated inhalational anthrax can reach 99%, with antibiotic treatment being largely unsuccessful if initiated after the onset of non-specific febrile symptoms. Naturally occurring and genetically engineered antibiotic resistances amplify the threat of weaponized spores and accentuate needs for improved treatments and methods for spore detection following an intentional release. (See Annu. Rev. Microbiol, JAMA, supra.)

A Mycobacterium tuberculosis infection begins with inhalation of the mycobacteria. The disease is contagious, deadly, and needs to be controlled promptly.

Mycobacterium bovis, which is transmitted by milk from infected cows, can also cause tuberculosis. (See Robins Pathologic Basis of Infectious Diseases, p. 349).

Corynebacterium diptheriae and Bordetella pertussis are both diseases that have previously all but vanished from most developed countries. However, these diseases are still found in developing countries, and, to a much lesser extent due to immigration, in developed countries. Corynebacterium diptheriae is a particularly vile and painful disorder, as the bacteria accumulate in the mucous linings and surface of the throat, causing growth, inflammation, and ultimately strangulation.

Bordetella pertussis is also a painful, strength wrenching bacterial infection, and causes the disease commonly known as whooping cough. Bordetella pertussis is not invasive but attaches to tracheal cilia inhibiting their function of mucus removal. The subsequent mucus accumulation leads to violent coughing. This disease is most dangerous to young children and infants, who already have small windpipes.

Streptococcus pyogenes and B-hemolytic are bacteria responsible for upper respiratory tract infections, scarlet fever, septicemia, and erysipelas. Regrettably, many cases of Streptococcus pyogenes, B-hemolytic are still found around the world, particularly in die less developed countries. Acute Streptococcus pyogenes infections may present as pharyngitis (strep throat), scarlet fever (rash), impetigo (infection of the superficial layers of the skin) or cellulitis (infection of the deep layers of the skin). Invasive, toxigenic infections can result in necrotizing fasciitis, myositis and streptococcal toxic shock syndrome. Patients may also develop immune-mediated post-streptococcal sequelae, such as acute rheumatic fever and acute glomerulonephritis, following acute infections caused by Streptococcus pyogenes. (© 2002 Kenneth Todar University of Wisconsin-Madison Department of Bacteriology)

Other bacteria that can cause a respiratory ailment include, but are not limited to, Staphylococcus aureus, Staphylococcus epidermidis, Klebsiella pheumoniae, and Pseudomonas aeruginosa.

K. pneumoniae infections are common in hospitals where they cause pneumonia characterized by emission of bloody sputum and urinary tract infections in catheterized patients (CDC Reports) at www.buddycom.com).

Klebsiella rhinoscleromatis is the causative agent of rhinoscleroma, a slowly progressive (months to years) mucosal upper respiratory infection that causes necrosis and occasional obstruction of the nasal passages. Another strain or variety of Klebsiella, Klebsiella ozaenae, can cause chronic. atrophic rhinitis (See, Harrison's Principles of Internal Medicine, 15^{th} Edition, p. 957).

Similarly, Proteus mirabilis may also be responsible for pneumonia, typified by fevers, chest pains, rates, chills, dyspnea, cough, and cyanosis.

An organism that has only been identified during the last twenty five years, and apparently is found primarily in developed countries, is L. pneumophilia, or, as it is commonly called, Legionnaires disease. Often found in ventilation systems, the disease tends to be fatal in older individuals.

A variety of species of Acinetobacter, and in particular A. baumanni, A. calcoaceticus, and Acinetobacter genospecies 3 and 13TU may cause nosocomial pneumonia.

Serratia marcesens is another bacteria that is spread through poor hygiene and poor sterile technique particularly in institutional settings. Exposure can result in lower respiratory tract infections. Sometimes exposure comes about due to a patient using respiratory equipment that has not been properly cleaned or sterilized.

Burkholderia cepacia and Stenotrophomonas maltophilia can cause pneumoniae and chronic lower respiratory tract infections.

Burkholderia pseudomallei, a bacteria similar to Pseudomonas that is found primarily in Southeast Asia, can cause meliodosis, a disease that may resemble bronchitis and pneumonia, which can also have the symptoms of tuberculosis.

Coxiella burnetti produces Q fever, which produces a fever and pneumonia (see Robbins, Pathologic Basis of Disease, 6^{th} Edition, 1999, p. 384).

Additional flora include P. Melaninogenica, Fusobacterium spp.

Other bacteria that may cause “anaerobic” pneumoniae include Prevotella spp., F. nucleatum, Peptostreptococcus spp., and Bacteroides spp. These bacteria are most responsive to parenteral treatment (see below).

While many of the above listed bacteria no longer respond well to antibiotics, each of the bacteria has as enemies an almost infinite number of bacteriophage that genetically code for enzymes that destroy that specific bacteria being treated, such that these enzymes will destroy the cell walls of the targeted organism. Put in the appropriate mode for delivery, the therapeutic agent forms a rapid and effective method of killing the specific bacteria while causing no harm to the host organism or to the natural bacterial flora.

The therapeutic agent or composition can contain any or all of these lytic enzymes specifically genetically coded for by phages specific for each bacteria discussed above, and may contain other lytic enzymes for other pathogenic bacteria that are present.

The composition which may be used for the prophylactic and therapeutic treatment of, for example, a strep infection includes a pharmaceutically effective amount of the lysin enzyme and a means of application (such as a carrier system or an oral delivery mode) to the mucosal lining of the oral and nasal cavity, such that the enzyme is put in the carrier system or oral delivery mode to reach the mucosal lining.

While an “unmodified” or “unaltered” phage associated lytic enzyme may be used for treatment of bacteria that cause respiratory infections, it may be preferred that a shuffled or chimeric lytic enzyme be used, possibly with a holin protein.

The preferred methods of treating an upper respiratory infection is to apply the lytic enzyme orally, although for more serious infections and for some lower respiratory infections, a parental application (discussed below) of the enzyme will be necessary.

Prior to, or at the time the lysin enzyme is put in the carrier, system or oral delivery mode, it is preferred that the enzyme be in a stabilizing buffer environment for maintaining a pH range of between about 4.0 and about 9.0, more preferably between about 5.5 and about 7.5 and most preferably at about 6.1.

The stabilizing buffer should allow for the optimum activity of the lysin enzyme. The buffer may be a reducing reagent, such as dithiothreitol. The stabilizing buffer may also be or include a metal chelating reagent, such as ethylenediaminetetraacetic acid disodium salt, or it may also contain a phosphate or citrate-phosphate buffer. Other appropriate buffers may be used.

Means of application include, but are not limited to direct, indirect, carrier and special means or any combination of means. Direct application of the enzyme nay be by nasal sprays, nasal drops, nasal ointments, nasal washes, nasal injections, nasal packings, bronchial sprays and inhalers, or indirectly through use of throat lozenges, or through the use of mouthwashes or gargles, or through the use of ointments applied to the nasal nares, the bridge of the nose, or the face or any combination of these and similar methods of application. The forms in which the lysin enzyme may be administered include but are not limited to lozenges, troches, candies, injectables, chewing gums, tablets, powders, sprays, liquids, ointments, and aerosols. The mode of delivery may also be a form of carrier, where possible.

The lozenge, tablet, or gum into which the enzymes are added may contain sugar, corn syrup, a variety of dyes, non-sugar sweeteners, flavorings, any binders, or combinations thereof. Similarly, any gum based products may contain acacia, carnauba wax, citric acid, corn starch, food colorings, flavorings, non-sugar sweeteners, gelatin, glucose, glycerin, gum base, shellac, sodium saccharin, sugar, water, white wax, cellulose, other binders, and combinations thereof.

Lozenges may further contain sucrose, corn starch, acacia, gum tragacanth, anethole, linseed, oleoresin, mineral oil, and cellulose, other binders, and combinations thereof. In another embodiment of the disclosure, sugar substitutes are used in place of dextrose, sucrose, or other sugars.

The enzyme may also be placed in a nasal spray, wherein the nasal spray is or may be the carrier. The nasal spray can be a long acting or timed release spray, and can be manufactured by means well known in the art. A bronchial spray or inhalant may also be used, so that the phage enzyme may reach further down into the bronchial tract, including into the lungs. Any other device that can carry the enzyme into the lungs and bronchial tract is acceptable.

Any of the carriers for the lytic enzymes may be manufactured by conventional means. However, it is preferred that any mouthwash or similar type products not contain alcohol to prevent denaturing of the enzyme. Similarly, when the lytic enzymes are being placed in a cough drop, gum, candy or lozenge during the manufacturing process, such placement should be made prior to the hardening of the lozenge or candy but after the cough drop or candy has cooled somewhat, to avoid heat denaturation of the enzyme. The enzyme can also be used to coat the outside of the lozenge, but the dosage of the enzyme may or may not be enough in such a coating, depending on the active units/ml of coating.

The enzyme may be added to these substances in a liquid form or in a lyophilized state, whereupon it will be solubilized when it meets body fluids such as saliva. Similarly, during certain phases of manufacture, and in certain carriers, the enzymes will exist in a solubilized form The enzyme may also be in a micelle or liposome whereupon it is then put in a carrier or in a delivery system. Enzymes may also be placed onto solid applicators such as tampons with the appropriate pharmaceutical components to allow for the slow release of enzymes into the appropriate site. Enzymes may also be placed on bandages to aid in the treatment of wounds and burns. Enzymes can also be placed in creams with the appropriate pharmaceutical compound to treat local skin diseases. The effective dosage rates or amounts of the lytic enzyme(s) to treat the infection will depend in part on whether the lytic enzyme will be used therapeutically or prophylactically, the duration of exposure of the recipient to the infectious bacteria, the size and weight of the individual, etc. The duration for use of the composition containing the enzyme may also depend in part on whether the use is for prophylactic purposes, wherein the use may be hourly, daily or weekly, for a short time period, or whether the use will be for therapeutic purposes wherein a more intensive regimen of the use of the composition may be needed, such that usage may last for hours, days or weeks, and/or on a daily basis, or at timed intervals during the day. Any dosage form employed should provide for a minimum number of units for a minimum amount of time. The concentration of the active units of enzyme(s) believed to provide for an effective amount or dosage of enzyme may be in the range of about 100 units/ml to about 100,000 units/ml of fluid in the wet or damp environment of the nasal and oral passages, and possibly in the range of about 100 units/ml to about 10,000 units/ml. In some circumstances, the units/ml of enzyme may be as high as 500,000 units/ml, and possibly several million units/ml, but the latter is best administered parenterally. More specifically, time exposure to the active enzyme units may influence the desired concentration of active enzyme units per ml. It should be noted that carriers that are classified as “long” or “slow” release carriers (such as, for example, certain nasal sprays or lozenges) could possess or provide a lower concentration of active (enzyme) units per ml, but over a longer period of time, whereas a “short” or “fast” release carrier (such as, for example, a gargle) could possess or provide a high concentration of active (enzyme) units per ml, but over a shorter period of time. The amount of active units per ml and the duration of time of exposure depends on the nature of infection, whether treatment is to be prophylactic or therapeutic, and other variables.

While this treatment may be used in any mammalian species, the preferred use of this product is for a human. Carriers for animal uses (and even humans)) could be as a food additive, being added to hay, grasses, watermelons, pumpkins (an elephant's favorite food), water sources, pills, candies, and beef, pork, chicken and fish for meat eaters. These enzymes can be added to or incorporated into dog and cat foods, which are typically beef and beef byproducts, fish, horsemeat, and chicken. Doses may vary, depending on the animal, on the animal's size, and the severity of the illness. All of the uses of the enzymes for different diseases in this patent may be applied to animals as well as to humans.

While these enzymes in the respective delivery systems and carriers can be used for therapeutic treatment of illnesses, they can also be used as a prophylactic treatment, administered to people (and animals) who may or may not have or have the potential to come in contact with carriers of the bacteria.

For the most serious infections, the lytic enzyme, holin protein, chimeric enzyme, shuffled enzyme, and combinations thereof may be administered parenterally using an effective amount of a therapeutic agent, the therapeutic agent comprising at least one lytic enzyme genetically coded for by a bacteria infected with a bacteriophage specific for said bacteria, and a carrier for delivering the lytic enzyme to the site of the infection.

More than one lytic enzyme may be introduced into the infected body at a time.

A number of different methods may be used to introduce the lytic enzyme(s) parenterally. These methods include introducing the lytic enzyme intravenously, intramuscularly, subcutaneously, subdermally, and intrathecally. For treating upper and lower respiratory ailments, intravenous treatment may be best. The therapeutic agent should comprise the appropriate and effective amount of the lytic enzyme(s) (holin lytic enzyme, chimeric lytic enzyme and/or shuffled lytic enzyme) in combination with a carrier comprising distilled water, a saline solution, albumin, a serum, or any combination thereof. More specifically, solutions for infusion or injection may be prepared in a conventional manner, e.g. with the addition of preservatives such as p-hydroxybenzoates or stabilizers such as alkali metal salts of ethylene-diamine tetraacetic acid, which may then be transferred into fusion vessels, injection vials or ampules. Alternatively, the compound for injection may be lyophilized either with or without the other ingredients and be solubilized in a buffered solution or distilled water, as appropriate, at the time of use. Non-aqueous vehicles such as fixed oils and ethyl oleate are also useful herein, although are usually not recommended for intravenous use. A straight intravenous solution with the enzyme in the appropriate solutions and buffers is best.

The carrier suitably contains minor amounts of additives such as substances that enhance isotonicity and chemical stability. Such materials are nontoxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, or immunoglobulins; hydrophilic polymers such as polyvinylpyrrolidone; glycine; amino acids such as glutamic acid, aspartic acid, histidine, or arginine; monosaccharides, disaccharides, and other carbohydrates including cellulose or its derivatives, glucose, mannose, trehalose, or dextrins; chelating agents such as EDTA; sugar alcohols such as mannitol or sorbitol; counter-ions such as sodium; non-ionic surfactants such as polysorbates, poloxamers, or polyethylene glycol (PEG); and/or neutral salts, e.g., NaCl, KCl, MgCl.sub.2, CaCl.sub.2, etc.

Glycerin or glycerol (1,2,3-propanetriol) is commercially available for pharmaceutical use. It may be diluted in sterile water for injection, or sodium chloride injection, or other pharmaceutically acceptable aqueous injection fluid, and used in concentrations of 0.1 to 100% (v/v), preferably 1.0 to 50% and more, but preferably about 20%.

The carrier vehicle may also include Ringer's solution, a buffered solution, and dextrose. solution, particularly when an intravenous solution is prepared.

The chimeric and/or shuffled lytic enzymes may be used in combination with other chimeric and shuffled lytic enzymes, holin proteins, other lytic enzymes, and other phage associated lytic enzymes which have not been modified or which are not “recombinant.”

While the most serious infectious cases should be treated in the hospital, it is possible for the patient to receive an appropriate enzyme solution by a portable pump that may be either a mechanical or electro-mechanical pump. In some cases, the patients may receive daily or two or more injections a day of the enzyme solution. Additionally, while intravenous is recommended for many patients, there may be subcutaneous, intramuscular and other forms of parental administration in other cases where the bacteria has infected other specific parts of the body.

It should also be noted that the lytic enzyme can be administered parenterally by means of a continuous drip, or by one or more daily injections.

If there has been either exposure or potential exposure of the individual either animal or human, to a bacteria, prophylactic treatment should begin as soon as possible after exposure. Prophylactic treatment can be the same treatment as that of therapeutic treatment. This should help prevent the spread of the bacteria and help the exposed party from becoming ill.

For treatment of infections of the upper respiratory tract, lozenges, troches, candies, injectants, chewing gums, tablets, powders, sprays, liquids, ointments, and aerosols, among others, may be used to deliver the lytic enzymes for the treatment of Serratio marcesens, Burholderia cepacia, Staphylococcus aureus, Staphylococcus epidermidis, Klebsiella pneumoniae, Pseudomonas aeruginosa, K. rhinoscleromatis, K. ozaenae, Proteus, mirabilis, L. pneumophilia, Acinetobacter, A. baumanni, A. calcoaceticus, Acinetobacter, genospecies, Stenotrophomonas maltophilia, Burholderia pseudomalei, Streptococcus pneumoniae, Hemophilus influenzae, Streptococcus A, Bacillus anthracis, Mycobacterium tuberculosis, Corynebacterium tuberculosis, Corynebaterium diphtheria, Coxiella burnetti, Bordetella pertussis, Streptococcus pyogenes, and B-hemolytic,

However, for certain bacterial infections, caused, for instance, by Klebsiella pneumoniae, B. anthracis, Streptococcus pneumoniae, Prevotella spp., F. nucleatum, Peptostreptococcus spp., and Bacteroides spp. and other forms of tuberculosis and pneumonias, parenteral treatment and inhalants which will go down the bronchial tubes and at least partly into the lungs are further recommended, in addition to using any of the modes of delivery listed above.

Additionally, another group of bacteria can start as oral infections, but end up causing endocarditis. This group includes Kingella kingae, Eikenella corrodens, several Haemophilus species, Actinobacillus actinomycetemcomitans, and Cardiobacterium hominis. While these organisms can be treated in the mouth, any sign of involvement of the heart must be treated promptlytby parenteral methods. As many cases of endocarditis begin in the dentists' chair after oral surgery, preventative treatment of these bacteria may begin before or after each oral surgery. Administration of the lytic enzymes for these bacteria may be in any form that allows delivery to the mouth, gums, and throat.

The dentist may also give an intravenous administration or injection into those patients at risk.

Additionally, in order to accelerate treatment of the bacterial illness, the therapeutic agent may further include at least one complementary agent which can also potentiate the bactericidal activity of the lytic enzyme. The complementary agent can be penicillin, synthetic penicillins bacitracin, methicillin, cephalosporin, polymyxin, cefaclor. Cefadroxil, cefamandole nafate, cefazolin, cefixime, cefmetazole, cefonioid, cefoperazone, ceforanide, cefotanme, cefotaxime, cefotetan, cefoxitin, cefpodoxime proxetil, ceftazidime, ceftizoxime, ceftriaxone, cefriaxone moxalactam, cefuroxime, cephalexin, cephalosporin C, cephalosporin C sodium salt, cephalothin, cephalothin sodium salt, cephapirin, cephradine, cefuroximeaxetil, dihydratecephalothin, moxalactam, loracarbef. mafate, chelating agents, streptomycin, erythromycin, chloramphenicol, numerous other antibiotics, and any combinations thereof in amounts which are effective to synergistically enhance the therapeutic effect of the lytic enzyme. It should be noted that virtually any antibiotic may be used as complementary agents for or with any use of the recombinant lytic enzymes.

While many or even most of the bacteria listed above can infect animals, and some of the same carriers can be used for animals to provide them with the, lytic enzymes used to kill the bacteria, it may be advantageous to put these lytic enzymes in appropriate carriers. Any appropriate pharmaceutically acceptable carrier may be used to deliver phage associated lytic enzymes.

Actinobacillus lignieresii is an opportunisitic invader of tongue tissue in cattle, small ruminants, and horses.

Strangles is an infectious horse disease caused by Streptococcus equi, subsp. equi. Symptoms include suppurative rhinits and lymphadenitis (mandibular and retropharyngeal with occasional hematogenous dissemination to internal organs. (McGavin et al, Thomson's Special Veterinary Pathology, 3^{rd} Ed. p. 135). S. equi is transmitted by feed, sweat, phlegm, air, etc. This nasal infection can lead to, or be characterized by, conjunctivitis, swelling of the lymph nodes, and can lead to bronchopneumoniae, laryngeal hemiplegia, facial paralysis, cellulitis, etc (see Thomson's, p. 136).

Bordetella bronchiseptica, Pasteurella multocida, and Haemophilus parasuis can cause atrophic rhinitis in pigs. It is believed that the combined infection of specific strains of B. bronchiseptica and some toxigenic strains of P. multocida [types D and A] ((McGavin et al, Thomson's Special Veterinary Pathology, 3^{rd} Ed. p. 138). Hence, in treating this disorder, it is best that phage associated lytic enzymes for both bacteria be used.

Acute bacterial rhinitis in dogs is caused by is caused by Bordetella bronchiseptica, E. coli and Pasteurella multocida.

Acute bacterial supperative rhinitis in cats is caused by Pasteurella multocida, Bordetella bronchiseptica, Streptococcus sp., and Mycoplasma felis. Infections caused by Mycoplasma felis can lead to an upper respiratory infection. These bacteria are normally a secondary infection following respiratory disease caused by feline herpes virus.

There are, of course, a number of other bacteria which infect parts of the respiratory tract of various animals.

Fusobacterium necrophorum infects feedlot cattle and cause lesions of the tongue, gingiva, cheeks, palate, and pharynx (See Thomson's p. 144).

Bordetella bronchiseptica can cause canine infectious tracheobronchitis.

Supperative bronchopneumonia in domestic animals may be caused in part by Pasteurella multocida, Bordetella bronchiseptica, Arcanobacterium, (Actinomyces) pyogenes, Streptococcus spp., and E. coli, among other bacteria.

Fibrinous bronchopneumonia in domestic animals may be caused by Pasteurella haemolytica, Haemophilus somnus, Actinobacillus pleuropneumoniae, and Mycoplasma mycoides.

Granulomatous pneumonia in animals may be caused by Mycobacterium bovis in ail animals and by Rhodococcus equi in foals (Thomson's p. 165).

While these animals may be treated with similar lytic enzymes and carriers as those of humans, it may be advisable to adapt some of the carriers for use by pigs, horses and cattle.

Pneumoniae in horses can be caused, in some circumstances, by Streptococcus spp., E. coli, Klebsiella pneumoniae, Rhodococcus equi,

P. haemolytica and P. multocida are associated with “shipping fever” in cattle. Shipping fever is also known as pneumonic asteurellosis, respiratory hemophilosis, and several other names.

For example, the lytic enzyme may be added to food slurries for these animals, such as to the “slop” that is fed to pigs, the hay, fruits, meats, and other nutrients fed to horses, and the grasses, hay, corn, and other grains fed to cattle. The enzymes can be added to water, with the appropriate buffers added, or it may be sprayed on the dried foods eaten by the animals.

Digestive Tract

Another use of a lytic enzyme is for the treatment of bacterial infections of the digestive tract.

Most gastrointestinal infections are a direct result of poor sanitation, allowing for the contamination of water and food. This contamination is usually in the form of fecal bacteria contaminating the water and food sources, and can be representative of inadequate or nonexistent sewage treatment.

Alternatively, poor personal hygiene before eating or when preparing food or drink, and/or failing to thoroughly cook food, can result in gastrointestinal infections that end in severe illness or tragedy. Throughout the United States, a country well known for its hygienic culture, many individuals get extremely ill each year from eating contaminated ground beef that has not been thoroughly cooked, thereby allowing bacteria in the meat to survive and thrive.

The proposed method for treating a bacterial infection of the digestive tract comprises administering a composition comprising both an effective amount of at least one lytic enzyme genetically coded for by a bacteriophage specific for the specific bacteria, and a carrier for delivering the lytic enzyme to the infected section of the digestive tract, wherein the specific lytic enzyme digests the cell wall of the bacteria for which it is specific. There are numerous different bacteria infections that cause a variety of disorders of the gastrointestinal system, for which there are a number of bacteriophage encoding for a lytic enzyme which can digest the cell wall of a specific bacteria causing the infections.

A brief discussion of the conditions and bacteria causing digestive maladies, and proposed methods of treatment, is in order.

Sialoadenitis (inflammation of the salivary glands) is often caused by Staphylococcus aureus or Streptococcus viridans. The bacterial version of Sialoadenitis may be treated by using an oral wash containing the lytic enzyme, shuffled lytic enzyme, chimeric lytic enzyme, holin proteins and combinations thereof in a carrier as previously described for treatment of oral infections.

Bacterial esophagitis is unusual, but can be caused by Lactobacillus and B-hemolytic streptococci in an immunocompromised host. In patients with profound granulocytopenia and patients with cancer, bacterial esophagitis is often missed because it is commonly present with other organisms, including viruses and fungi. In patients with AIDS, infection with Cryptosporidium or Pneumocystis carinii may cause nonspecific inflammation, and Mycobacterium tuberculosis infection may cause deep ulcerations of the distal esophagus. (Harrison's Principles of Internal Medicine 15th Edition.)

To treat these bacterial infections of the esophagus, lytic enzymes specific for the specific bacteria causing the infection of the esophagus are administered to the area of the infection. This may be done either by parenteral administration or by oral administration of the lytic enzyme(s). The oral administration may be by a liquid which may also contain bismuth subsalicylate, metronidazole, tetracycline, or ranitidine bismuth citrate, with claritlromycin or metronizadole, omeprazole, plus Clarithromycin plus Metronidazole or Amoxicillin. A carrier, such as a mucoadhesive, or a Xylocaine® gel, or some other methyl cellulose which coats the esophagus is most helpful.

Gastric ulcers can be a result of infection by H. pylori. H. pylori may be treated by using a phage associated lytic enzyme, such as the M13 phage, which specifically cleaves the cell wall of H. pylori. The enzyme, which may be in a liquid coating solution such as a mucoadhesive, or a Xylocaine® gel, or some other methyl cellulose, may preferably also contain bismuth subsalicylate, metronidazole, tetracycline, or ranitidine bismuth citrate, with clarithromycin or metronizadole, omeprazole, plus Clarithromycin plus Metronidazole or Amoxicillin. The phage associated lytic enzyme may also be administered parenterally for treating the H. pylori infection. This bacteria may also be treated by putting the appropriate lytic enzymes in a pill Any other stomach bacteria may also be treated by this method, using the lytic enzyme specific for the specific infectious bacteria.

Moving down the digestive tract, the small and large intestines are fertile ground for bacterial infections. For example, chronic diarrhea in a tropical environment is most often caused by infectious agents including G. lambia, Yersinia enterocolitica, C. difficile, Cryptosporidium parvum, and Cyclospora cayetanensis, often associated with tropical sprue. Other bacteria associated with tropical sprue include Klebsiella pneumoniae, Enterobacterobacter cloacae, and E. coli.

In secretory diarrhea, not limited to tropical sprue, Vibrio cholerae, E. coli, Bacillus cereus, and Clostridium perfringens have been implicated. These bacteria work by multiplying inside the mucous overlying the gut epithelium and releasing exotoxins causing the gut epithelium to secrete excessive volumes of watery diarrhea. In exudative diseases, the bacteria damage the mucosal epithelium of the afflicted individual. Shigella, Salmonella, and Campylobacter are often to blame. Strains of Shigella that can be responsible include Shigella dysenteriae, Shigella flexneri, Shigella boydii, and Shigella sonnei. More specifically, in bacterial enterocolitis, diarrheal illness induced by bacteria may be caused by a variety of bacterial species and several pathogenic mechanisms, including the ingestion of preformed toxins, present in contaminated food (Staphylococcus aureus, Vibrios, and Clostridium perfringens). (See p. 807, Cotran, et al, Robbins Pathologic Basis of Disease, 6^{th} edition, 1999).

To infect the organism, the bacteria need to adhere to the mucosal epithelial cells, replicate, and produce enterotoxins, and invade. (See p. 807, Cotran, et al, Robbins Pathologic Basis of Disease, 6^{th} edition, 1999).

Both V. cholerae enterotoxins and certain strains of E. coli that produce heat-labile and heat stable toxins can cause diarrhea, particularly in developing nations. (Again, citing Cotran, et al, Robbins Pathologic Basis of Disease, 6^{th} edition, 1999) Enteroinvasive E. coli and Shigella, as well as Salmonella, can invade the epithelial cells. Yersinia enterocolitica is also extremely dangerous. C. difficile can cause pseudomembranous colitis.

Additionally, some strains of E. coli can interact with intestinal mucosa by a variety of pathogenic mechanisms to produce gastroenteritis.

It should be noted that strains of Klebsiella cause a variety of abdominal infections similar to those caused by E. coli, but are usually less a causative agent than E. coli.

The different bacteria mentioned also may invade different sections of the different digestive organs. For example, while Shigella primarily infects the distal colon, V. cholerae and Clostridium perfringens infects the small intestine. Salmonella (including strains typhimurium and paratyphi, as well as Y. enterocolittica and Y. pseudotuberculosis also infect the colon. The use of antibiotics can facilitate the growth of C. difficile in the large intestine, which in turn causes a bacterial form of colitis.

Campylobacter, and in particular Campylobacter jejuni, are often found in unpasteurized milk or contaminated water. An under appreciated organism, Campylobacter is more often the culprit in food contamination than Salmonella, and can cause diarrhea, dysentery and enterocolitis, and septicemia. When inhabiting the lymph nodes, arthritic symptoms may occur.

Yersinia enterocolitica and Yersinia pseudotuberculosis for whose contamination results from poor hygiene, are intracellular bacteria that cause lesions in the ileum and colon.

Salmonella, and in particular Salmonella enteritidis and Salmonella typhimurium, among others forms of Salmonella, are common bacteria found as causal agents in water and food poisoning. Salmonella is the bacteria most commonly associated in chicken manufacturing. During infection, both Salmonella enteridis and Salmonella typhimurium infect primarily the ileum and the colon.

Bacterial overgrowth syndrome comprises a group of disorders with diarrhea, steatorrhea, and macrocytic anemia whose common feature is the proliferation of colon type bacteria within the small intestine. This bacterial proliferation is due to stasis caused by impaired peristalsis (i.e. function stasis), changes in intestinal anatomy (i.e. anatomic stasis) or direct communication between the small and large intestine. The manifestations of bacterial overgrowth syndromes are a direct consequence of the presence of increased amounts of a colonic-type bacterial flora, E. coli and Bacteroides, in the small intestine. (Harrison's, p. 1676).

Other bacterial infections of the digestive system may be caused by bacteria selected from the groups consisting of Listeria and Staphylococcus.

Lysteria monocytogenes can contaminate dairy products, processed meats, and chicken.

Food contamination caused by Staphylococcus aureus can lead to severe vomiting and diarrhea, due in part to the enterotoxins of the bacteria.

Edwardsiella infections, and in particular E. tarda (which is the only member of the genus known to cause disease in humans) cause gastroenteritis. Symptoms may range from watery diarrhea to an extreme case of colitis.

Whipple's disease is a chronic multisystem disease associated with diarrhea, steatorrhea, weight loss, arthralogia and central nervous system and cardiac problems that is caused by the bacteria Tropheryma whippelii.

It has been hypothesized that inflammatory bowel disease also has an infectious etiology. Specific agents that have received the most attention include Mycobacterium paratuberculosis, Paramyxovirus, and Helicobacter species. Multiple pathogens (e.g. Salmonella, Shigella sp. Campylobacter sp.) may initiate IBD by triggering an inflammatory response that the mucosal immune system may fail to control. (See 15^{th} Ed., Harrison's Principles of Internal Medicine, p. 957).

Hence, it may be advisable that the first step for treatment of these digestive diseases caused by a bacterial infection of the intestinal tract is to treat the bacteria possibly infecting the bowel. A stool sample may optionally be first tested to determine which, if any, bacteria is causing the problem. Often, based on the symptoms, the digestive disorders, any ongoing endemics and epidemics, and the local culture, it is possible to determine the bacteria causing the digestive illnesses without a stool sample. Treatment of the illnesses may commence using the appropriate phage associated lytic enzymes which are specific for the specific bacterial pathogen causing the illness. Unaltered, chimeric and shuffled lytic enzymes, along with holin proteins, may be used to treat all of these digestive illnesses, alone or in combination with the “natural” or unaltered lytic enzymes. As with the unaltered lytic enzyme, the chimeric and shuffled lytic enzymes may be done either by parenteral administration, oral administration, or by rectal administration, depending on the site and degree of the infection.

The lytic enzyme may be produced by standard techniques, such as by incorporating the genetic coding for the enzyme in a vector, which is introduced into a bacteria which can produce the enzyme without itself being lysed. Any other technique may be used for production. This applies to any and all uses of the enzyme in treating any illness. Fermentation type factories may be used to produce the enzymes en mass. These techniques are well known in the art.

The means for delivering the enzyme(s) is selected from the group consisting of suppositories, enemas, syrups or other similar solutions, enteric coated pills, and in the most severe cases, by parenteral administration, preferably administering the enzyme intravenously.

The solutions containing the lytic enzyme(s) may also contain or have a methylcellulose base as the inactive ingredient. They can also have a bismuth based syrups or coating solution. The oral administration may be by a liquid which may (preferably) also contain bismuth subsalicylate, metronidazole, tetracycline, or ranitidine bismuth citrate, with clarithromycin or metronizadole, omeprazole, plus Claritlromycin plus Metronidazole or Amoxicillin.

The term pill referring to a pill, capsule, capsid, or any similar type delivery structure.

The rectal suppository may best be used for delivering the drug to the colon and rectum. The suppository is known in the art, and is made of glycerin, fatty acids, and similar type substances that dissolve at body temperature. As the suppository dissolves, the phage associated lytic enzyme(s) will be released.

As noted above, the lytic enzyme can be administered parenterally by means of daily injections, or they can be given by an IV drip. The decisions as to dosages, number of units/ml, carrier, whether a patient should be hospitalized or be treated as an outpatient, given the enzyme(s) parenterally, or a number of other variables are all dependent on the nature of the symptoms, the degree and type of exposure, the bacteria to which the patient was exposed, the general health of the patient, whether the patient is human (animals can be treated by these methods), or any number of factors.

These proposed modes of delivery can be made by conventional methods. However, the only difference in their manufacture is that the enzyme being placed in the carrier must not be allowed to permanently denature. The enzyme should be incorporated into a carrier which does not contain alcohol, and which has been cooled to a temperature that will not cause the permanent denaturing of or damage to the enzyme. The enzyme may be incorporated in a lyophilized state, or may be incorporated in an liposome before being placed in the suppository, syrup or enteric coasted pill. The enzyme placed in the composition or carrier should be in an environment having a pH which allows for activity of the lytic enzyme. To this end, the pH of the composition is preferably kept in a range of between about 2 and about 11. and even more preferably at a pH range of between 5.5 and 7.5. As described above with die other lytic enzyme, the pH can be moderated by the use of a buffer. The buffer may contain a reducing agent, and more specifically dithiothreitol. The buffer may also be a metal chelating reagent, such as ethylenediaminetetracetic disodium salt or the buffer may contain a citrate-phosphate buffer. As with all compositions described in this patent, the composition may further include a bactericidal or bacteriostatic agent as a preservative.

In all of the illnesses being treated discussed in this patent and in all illnesses to which this (these) enzyme(s) may be administered, and in all of the medications being given, more than one enzyme may be in the preparation being administered.

The lytic enzyme(s) preferably are present in a concentration of about 100 to about 500,000 active enzyme units per milliliter of fluid in the wet environment of the gastrointestinal tract, preferably about 100 to about 100,000 active enzyme units per milliliter of fluid, and preferably present in a concentration of about 100 to about 10,000 active enzyme units per milliliter of fluid in the wet environment of the gastrointestinal tract. However, the active enzyme units per milliliter may be as high as 5-10 million units in extreme or more severe cases.

Another extremely serious bacterial infection in addition to those mentioned above is bacterial peritonitis. Peritonitis is often caused by some external abdominal trauma, such as a puncture wound, or by an internal trauma, such as diverticulitis, ulcers; appendicitis, etc. While E. coli is the most common bacterial culprit in peritonitis, other bacterial agents include C. perfringens, enterococci, hemolytic streptococci and Staphylococcus aureus. It is critical that bacterial peritonitis be treated quickly, as gangrene or septicemia, may set in, followed by death.

To treat these bacterial infections, lytic enzymes specific for the specific bacteria causing the infections of the ileum, colon and small intestine may administered to the area of the infection. This may be done by parenteral administration, oral administration, or by the other means listed above. When treating peritonitis, parenteral administration is probably the best, although not die only means of treatment. Cleaning and surgically dressing the wound, as well as providing any oral administration of the enzyme(s), is advantageous.

When a parenteral administration of the lytic enzyme(s) is(are) needed, the administration may take place in a hospital, or, if the patient so desires and the doctors allow for it, a mechanical or mechanoelectrical pharmaceutical injection pack may be worn by the patient 24/7. The pack may be small and innocuous, or, the larger electromechanical pack may be worn around the waist. These packs may be used for most parenteral administration of enzymes, when the patient needs to be mobile, or when a hospital stay is not necessary. Home care providers can change the medication as required or the patient may be able to change the packs themselves. The patient can also have the intravenous administration of these drugs performed in their homes or other settings other than that of a hospital.

These enzymes can also be administered nutritionally. Each and every one of these lytic enzymes, chimeric lytic enzymes, shuffled lytic enzymes, and combinations of enzymes can be included in a food product, be it liquid or be it foodstuff. This is particularly helpful when treating animals, although humans may also be treated this way. In the case of those enzymes needed in the gut, the enzymes can be enterically coated or they can be included in a liposome. This applies to all of the enzymes, not just those for treating digestive disorders.

The enzymes may also be used prophylactically. When an individual has potentially been exposed to any of these or other pathogens in water or food, treatment may begin to prevent the growth and spread of the bacteria throughout the body, using any of the methods described above. Where it is known that pathogens are ubiquitous in the food or water of an area, or where sanitation is weak, it is best to give these enzymes prophylactically in all forms necessary (enteric pills, suppositories, solutions, etc.) to avoid digestive disorders. Prophylactic treatment is most important during epidemics, such as cholera epidemics, etc.

It is to be remembered that each bacteria is susceptible to numerous bacteriophages, each coding for a lytic enzyme that can lyse that specific bacteria. Any individual, variety, or combination of lytic enzymes, chimeric lytic enzymes, and/or shuffled lytic enzymes may be used.

Additionally, further techniques may be used to prevent the spread of the listed bacteria either into the food chain or onto the food directly.

Lytic enzymes and their modified forms can be used along the entire food processing chain in place of antibiotics to prevent dangerous infectious bacteria from; growing where antibiotics have not, or cannot, be used.

The method for treating food stuffs comprises treating the food stuffs with an anti-infection agent comprising an effective amount of at least one lytic enzyme produced by a bacteria infected with a bacteriophage specific for the bacteria, a holin protein, chimeric enzyme, shuffled enzyme, or combination thereof. More specifically, the lytic enzyme may be either supplemented by chimeric and/or shuffled lytic enzymes, or may be itself a chimeric and/or shuffled lytic enzyme. Similarly, a holin protein may be included, which may also be a chimeric and/or shuffled lytic enzyme.

Each and every one of the lytic enzymes, shuffled lytic enzymes, chimeric lytic enzymes, and combinations thereof may be put in any and all liquids and used in feeding humans and animals. Eggs may be dipped into or sprayed with solutions of the enzymes, as may chickens on the assembly line. Chicken and beef carcasses can similarly be dipped or sprayed with an enzymatic solution to kill die targeted bacteria. Juices, milk, water, wines, and other liquids may also be treated with the lytic enzyme.

Animal feeds can be either “dry” or “wet.” It is quite common that the animal feed is in the form of a thick slurry. In those instances, prior to feeding the animals, at least one lytic enzyme, a holin protein, chimeric enzyme, shuffled enzyme, or combinations thereof is added and mixed into the slurry. The enzyme(s) can be lyophilized or dehydrated. However, the lytic enzyme(s) added can also be in a carrier. Alternatively, during the processing of the feed stock, the feed can be bathed in a lytic enzyme bath, prior to packaging or prior to use. The feed can also be sprayed after it is placed in the feeding pen or trough.

The lytic enzyme or its modified form may also be dusted onto the carcass in a powder, as described above.

The carrier for the enzyme(s) may be water, an oil immersion, micelles, micelles in water or oil, liposomes, liposome in oil or water, combinations thereof, or any other convenient carrier. The enzyme(s) may be encapsulated in a carbohydrate or starch like structure, or the micelles or liposomes may be encapsulated by a starch or carbohydrate type structure. The carrier may also be in the form of a powder. The taste and texture of the carrier should be pleasing to the animal, so that the animal does not reject the food.

Sneeze guards, and salad bars may similarly be similarly sprayed with a lytic enzyme solution. If so desired, the individual vegetables and ingredients of the salad bar can be pre treated or pre dipped with the enzyme solution.

All containers, pots, pans, utensils (knives, forks, spoons, ladles, spatulas, etc), kitchen surfaces, milking machines, and food handling surfaces can be accordingly treated.

Furthermore, botulism, caused by Clostridium botulinum, may be prevented or limited by the addition of the lytic enzymes for C. botulinum, to products and containers to be sealed, although to be most effected, the C. botulinum must be in a vegetative state.

Additionally, appropriate antibiotics may be included with the lytic enzymes, although the antibiotics should be narrow in scope so as not to eliminate desirable bacteria, which would thus defeat one of the purposes of this disclosure.

Just as humans get bacterial infections and can be treated with them by the above methods, animals and fowl can also be treated using similar methods. In some cases, the lytic enzyme can be put in different carriers more suitable for the animal or fowl being treated, such as in the foods being eaten.

Salmonella typhisuis causes sialoadenitis (inflammation of the salivary glands) in pigs (See McGavin, Thomson's Special Veterinary Pathology, 3^{rd} Edition, 2001 p. 11). This may be treated by adding food supplements containing the lytic enzyme, spraying the enzyme on food, adding the enzyme to a food slurry, given in candies, sweet treats, included in both food slurries (i.e., “slop”), sprayed on dry foods, etc.

Streptococcus bovis and Lactobacillus spp., brought about by highly fermentable carbohydrate rich feed, results in the production of lactic acid which damages the ruminal mucosa in cattle (See Thomson's Special Veterinary Pathology, p. 18) Orally or parenterally administering the lytic enzymes by any of the delivery modes suggested for stomach ailments, along with a change in diet, will alleviate the spread of these infectious agents.

However, secondary infections may lead to liver damage caused by Actinomyces pyogenes and Fusobacterium necrophorum. These bacteria can best be treated by parenteral administration of lytic enzymes specific for these bacteria.

C. perfringens can cause acute gastric dilation in horses, often after feeding in the pasture. While this disease in horses is primarily limited to Scotland, England, and Sweden (Thomson's Special Veterinary Medicine, p. 22), C. perfingnes type A is found in captive monkeys. Where possible, the lytic enzymes may be administered by enteric coated pills and parenteral administration, along with stomach lining coating delivery modes, including bismuth based delivery modes, are among the best treatments of this illness. Additionally, the lytic enzymes can be put into the horse feed, or the monkey feed, and can be added to Purina's Horse Chow and Purina's Monkey Chow. It can be added to all other foods monkeys and horses eat, including meats, vegetables, fluids, fruits, hay (horses), etc. There are five types of Clostridium perfringens: type A, type B, type C, type D, and type E.

C. perfringens type C affects calves, and foals during the first few days of life, and piglets during the first eight hours of life Clostridium D affects fattening sheep, goats and calves.

C. piliformis causes what is known as Tyler's disease in many mammalian species. This bacteria affects the liver, after first infecting the ileum cecum or colon. (Thomson's Special Veterinary Pathology, 3^{rd} edition, p. 46).

Acute phlegomonous gastritis occurs in dogs and is the result of infection of the gastric wall by bacteria, including but not limited to streptococci, staphylococci, E. coli, Proteus vulgaris, or Clostridium perfringens. (Thomson's Special Veterinary Pathology, p. 24).

Hemorrhagic abomasitis with submucosal emphysema of sheep and cattle, a disease caused by Clostridium septicum is spread by contaminated food. Probably one of the easiest ways of controlling this disease is to treat the food source with phage associated lytic enzymes specific for the specific bacteria.

C. perfringens and pathogenic strains of E. coli may infect the intestinal tract of neonatal pigs, calves and lambs. C. Perfringens infects foals as well.

In some cases, newborn calves, lambs and occasionally foals that have not received sufficient colostral immunity suffer from septicemic colibacillosis. Symptoms of the disease include fibrous arthritis, ophthalmitis, serositis, meningitis, and white spouted kidneys. The bacteria gain entry to the body through the respiratory system, oral cavity or umbilicus and become septicemic (Thomson's Special Veterinary Pathology, p. 42,),

Additionally, postweaning colibacillosis (of pigs) is caused by a hemolytic E. coli, and enteroinvasive colibacillosis of human beings, lab animals and occasionally cattle and pigs are all caused by E. coli, and may result in hemorrhagic colitis and sometimes the hemolytic-uremic syndrome (see Thomson's Special Veterinary Pathology, p. 43).

These illnesses may be treated by a phage associated lytic enzyme specific for E. coli. For treatment of the bacterial infection, as with all treatments involving a phage associated lytic enzyme, a pharmaceutically acceptable carrier delivering an effective amount of the enzyme is to be used. In the treatment of E. coli infections, administration may be parenterally, by means of adding to the feed of the animal (including milk or formula being given newborns), enteric coated pills, liquid coatings, and similar type delivery means.

As in humans, Salmonella bacteria are another major cause of animal stomach and intestinal related illnesses. Contaminated food and water sources are most often to blame for this infection.

Enteric Salmonella infections caused by Salmonella typhimurium are often found in horses, cattle and pigs.

Salmonella cholerasuis can cause septicemia in calves, foals, and pigs.

Treatment is similar to that for E. coli infections, except that phage associated lytic enzymes specific for Salmonella strains are used.

Cattle, calves, pigs and dogs can get intestinal tuberculosis and can become infected with Mycobacterium tuberculosis and Mycobacterium bovis. These bacteria can enter through respiratory or gastrointestinal means. Horses also contract M. bovis.

Similarly, Mycobacterium avium infections can cause similar symptoms in the intestinal tract as can Mycobacterium tuberculosis, particularly in horses.

Cattle, sheep, goats, and other ruminants are subject to infection by Mycobacterium partuberculosis from feces contaminated soil.

As pigs and other ruminants may be infected by certain bacterial agents, horses can also be infected.

Swine dysentery, generally confined to the large intestine of pigs, is caused by Brachyspira hyodysenteriae, acting synergistically with anaerobic colonic flora such as Fusobacterium necrophorum or Bacteroides vulgatus. Weaning pigs 8 to 14 weeks old are usually affected, and the disease spreads rapidly through a herd (Thomson's Special Veterinary Pathology, p. 52).

Lawsonia enteritis in pigs is caused by a bacteria now called Lawsoniam and is typified by proliferative enteropathy, proliferative ileitis, intestinal adenomatosis, distal ileal hypertrophy, and terminal ileitis (see Thomson's Special Veterinary Pathology, p. 53).

While Rhodococcus equi is part of the normal bacterial flora of equine intestines, under certain condition, the bacteria can become pathogenic and can cause ulcerative enterocolitis.

As noted previously, these bacteria may be treated by unaltered phage associated lytic enzymes specific for the specific bacteria infecting the host, chimeric lytic enzymes, shuffled lytic enzymes, holin proteins, or combinations thereof.

The dosage range may be from about 10,000 units/ml to about 500,000 units/ml with dosages up to or greater than 10,000,000 units/ml.

Dosages for treatment of the illnesses may be administered by parenteral administration, oral administration, or by rectal administration. Depending on the site and degree of the infection, treatment may be made by pills, gel tabs, enteric coatings, and suppositories. For the most serious cases, the enzymes and/or holin proteins may be administered parenterally, including intravenous, intramuscular, or even intrathecally.

Alternatively, as noted previously, the appropriate unaltered, chimeric, and shuffled lytic enzymes and/or the holin protein may be added to food slurries, liquids, moist foods, dry foods (by adding lyophilized enzyme), etc. The enzyme(s) may be added in a lyophilized form or in a hydrated form. More than one enzyme may be added. Two or more enzymes genetically coded by different phages specific for the same bacterial species may be added. Similarly, two or more enzymes genetically coded by different phages specific for different bacterial species may be added.

Additionally, the appropriate holin protein may be added to the various carriers or foods.

Where necessary, the appropriate buffers, as described previously, can be used. As with human use, supplemental antibiotics, buffers, solutions, bismuth solutions, etc. may be used.

Dermatological Infections

Another composition and use of the lytic enzyme is for the therapeutic or prophylactic treatment of bacterial infections of burns and wounds of the skin.

Staphylococcus aureus, a very common bacteria, can cause skin infections. Typical symptoms of a Staphylococcus infection include carbuncles, impetigo, boils, etc. Staphylococcus infections tend to be opportunistic, and frequently infect the skin after a major trauma, such as severe burns, or deep cuts. Many Staphylococcus infections are acquired during hospital stays. In addition to Staphylococcus aureus, Staphylococcus epidermidis can also cause skin infections. In severe cases, cellulitis may result from Staphylococcus aureus infections, which, if untreated, can lead to death. Cellulitis may also be caused by Klebsiella, normally in a compromised tissue. Klebsiella is also the causative agent of many surgical site and other soft tissue infections.

Another bacteria which may cause an anaerobic form of cellulitis (or myonecrosis) is Clostridium, and specifically Clostridium perfringens (welchii) and Clostridium septicum. This form of skin infection and death is known as gas gangrene and is extremely dangerous.

Proteus mirabilis can infect wounds, causing pus, pain, inflammation, and erythema.

There are many other bacteria that can infect the skin.

Propionibacterium causes acne in adolescents.

Pseudomonas aeruginosa is a major cause of dermatological infections of burns and wounds in hospitals.

Certain strains of Streptococcus may also be responsible for cellulitis and certain skin infections. One, of the more severe strains of bacteria is Streptococcus fasciae. This strain of bacteria can rapidly destroy the tissue of the skin.

The various Enterobacter species (E. cloacae, E. aerogenes, E. agglomerans, E. sakazakii, and E. gergoviae) can also infect postoperative wounds, burns, skin ulcers and cuts.

Similarly, the various Acinetobacter species, including A. baumannii, A. calcoaceticus, and Acinetobacter genospecies 23 and 13Tu can infect the skin, particularly when the skin has been compromised by burns.

S. marcescens often infects surgical wounds.

Wounds can also become infected by E. tarda.

Similarly, B. cepaciae can cause surgical and burn wound infections.

Propionibacterium acne can also cause acne.

The most frequently isolated (anaerobic) organisms from skin and soft tissue infections include Bacteroides spp. Peptostreptococcus spp., enterococci, Clostridium spp. and Proteus spp. Anaerobic bacteria tend to cause a higher rate of fever, foul-smelling lesions, gas in the tissues, and visible foot ulcers.

Yaws is caused by Treponema pertenue and is characterized by topical lesions.

Peptostreptococcus and Bacteroides spp. can also cause necrotizing fasciitis. The various species of Peptostreptococcus include but are not limited to P. intermedius, P. micros, P. magnus, P. asaccharolyticus, P. anaerobius, and P. prevotii.

The compositions for treating bacterial infections of the skin comprise an effective amount of at least one lytic enzyme produced by a bacteria infected with a bacteriophage specific for the bacteria and a carrier for delivering at least one lytic enzyme to the wounded skin. The lytic enzyme(s) used for the topical treatment of burns may be either supplemented by chimeric and/or shuffled lytic enzymes, or may themselves be chimeric and/or shuffled lytic enzymes. Similarly, a holin protein may be included, which may also be a chimeric and/or shuffled lytic protein. The mode of application for the lytic enzyme includes a number of different types and combinations of carriers which include, but are not limited to an aqueous liquid, an alcohol base liquid, a water soluble gel, a lotion, an ointment, a nonaqueous liquid base, a mineral oil base, a blend of mineral oil and petrolatum, lanolin, liposomes, protein carriers such as serum albumin or gelatin, powdered cellulose carmel, and combinations thereof. A mode of delivery of the carrier containing the therapeutic agent includes but is not limited to a smear, spray, a time-release patch, a liquid absorbed wipe, and combinations thereof. The lytic enzyme may be applied to a bandage either directly or in one of the other carriers. The bandages may be sold damp or dry, wherein the enzyme is in a lyophilized form on the bandage. This method of application is most effective for the treatment of burns.

The carriers of the compositions of the present disclosure may comprise semisolid and gel-like vehicles that include a polymer thickener, water, preservatives, active surfactants or emulsifiers, antioxidants, sun screens, and a solvent or mixed solvent system. U.S. Pat. No. 5,863,560 (Osborne) discusses a number of different carrier combinations which can aid in the exposure of the skin to a medicament.

Polymer thickeners that may be used include those known to one skilled in the art, such as hydrophilic and hydroalcoholic gelling agents frequently used in the cosmetic and pharmaceutical industries. Preferably, the hydrophilic or hydroalcoholic gelling agent comprises “CARBOPOL.RTM.” (B. F. Goodrich, Cleveland, Ohio), “HYPAN.RTM.” (Kingston Technologies, Dayton, N.J.), “NATROSOL.RTM.” (Aqualon, Wilmington, Del.), “KLUCEL.RTM.” (Aqualon, Wilmington, Del.), or “STABILEZE.RTM.” (ISP Technologies, Wayne, N.J.). Preferably, the gelling agent comprises between about 0.2% to about 4% by weight of the composition. More particularly, the preferred compositional weight percent range for “CARBOPOL.RTM.” is between about 0.5% to about 2%, while the preferred weight percent range for “NATROSOL.RTM.” and “KLUCEL.RTM.” is between about 0.5% to about 4%. The preferred compositional -weight percent range for both “HYPAN.RTM.” and “STABILEZE.RTM.” is between about 0.5% to about 4%. CARBOPOL.RTM.” is one of numerous cross-linked acrylic acid polymers that are given the general adopted name carbomer. These polymers dissolve in water and form a clear or slightly hazy gel upon neutralization with a caustic material such as sodium hydroxide, potassium hydroxide, triethanolamine, or other amine bases. “KLUCEL.RTM.” is a cellulose polymer that is dispersed in water and forms a uniform gel upon complete hydration. Other preferred gelling polymers include hydroxyethylcellulose, cellulose gum, MVE/MA decadiene crosspolymer, PVM/MA copolymer, or a combination thereof.

Preservatives may also be used in this disclosure and preferably comprise about 0.05% to 0.5% by weight of the total composition. The use of preservatives assures that if the product is microbially contaminated, the formulation will prevent or diminish microorganism growth. Some preservatives useful in this disclosure include methylparaben, propylparaben, butylparaben, chloroxylenol, sodium benzoate, DMDM Hydantoin, 3-Iodo-2-Propylbutyl carbaniate, potassium sorbate, chlorhexidine digluconate, or a combination thereof.

Titanium dioxide may be used as a sunscreen to serve as prophylaxis against photosensitization. Alternative sun screens include methyl cinnamate. Moreover, BHA may be used as an antioxidant, as well as to protect ethoxydiglycol and/or dapsone from discoloration due to oxidation. An alternate antioxidant is BHT.

Pharmaceuticals for use in all embodiments of the disclosure include antimicrobial agents, anti-inflammatory agents, antiviral agents, local anesthetic agents, corticosteroids, destructive therapy agents, antifungals, and antiandrogens. In the treatment of acne, active pharmaceuticals that may be used include antimicrobial agents, especially those having anti-inflammatory properties such as dapsone, erythromycin, minocycline, tetracycline, clindamycin, and other antimicrobials. The preferred weight percentages for the antimicrobials are 0.5% to 10%. Local anesthetics include tetracaine, tetracaine hydrochloride, lidocaine, lidocaine hydrochloride, dyclonine, dyclonine hydrochloride, dimethisoquin hydrochloride, dibucaine, dibucaine hydrochloride, butambenpicrate, and pramoxine hydrochloride. A preferred concentration for local anesthetics is about 0.025% to 5% by weight of the total composition. Anesthetics such as benzocaine may also be used at a preferred concentration of about 2% to 25% by weight.

Corticosteroids that may be used include betamethasone dipropionate, fluocinolone actinide, betamethasone valerate, triamcinolone actinide, clobetasol propionate, desoximetasone, diflorasone diacetate, amcinonide, flurandrenolide, hydrocortisone valerate, hydrocortisone butyrate, and desonide at concentrations of about 0.01% to about 1.0% by weight. Preferred concentrations for corticosteroids such as hydrocortisone or methylprednisolone acetate are from about 0.2% to about 5.0% by weight.

Destructive therapy agents such as salicylic acid or lactic acid may also be used. A concentration of about 2% to about 40% by weight is preferred. Cantharidin is preferably utilized in a concentration of about 5% to about 30% by weight. Typical antifungals that may be used in this disclosure and their preferred weight concentrations include: oxiconazole nitrate (0.1% to 5.0%), ciclopirox olamiine (0.1% to 5.0%), ketoconazole (0.1% to 5.0%), miconazole nitrate (0.1% to 5.0%), and butoconazole nitrate (0.1% to 5.0%). For the topical treatment of seborrheic dermatitis, hirsutism, acne, and alopecia, the active pharmaceutical may include an antiandrogen such as flutamide or finasteride in preferred weight percentages of about 0.5% to 10%.

Typically, treatments using a combination of drugs include antibiotics in combination with local anesthetics such as polymycin B sulfate and neomycin sulfate in combination with tetracaine for topical antibiotic gels to provide prophylaxis against infection and relief of pain. Another example is the use of minoxidil in combination with a corticosteroid such as betainethasone diproprionate for the treatment of alopecia ereata. The combination of an anti-inflaminatory such as cortisone with an antifungal such as ketoconazole for the treatment of tinea infections is also an example.

In one embodiment, the disclosure comprises a dermatological composition having about 0.5% to 10% carbomer and about 0.5% to 10% of a pharmaceutical that exists in both a dissolved state, and a micro particulate state. The dissolved pharmaceutical has the capacity to cross the stratum corneuin, whereas the micro particulate pharmaceutical does not. Addition of an amine base, potassium, hydroxide solution, or sodium hydroxide solution completes the formation of the gel. More particularly, the pharmaceutical may include dapsone, an antimicrobial agent having anti-inflainmatory properties. A preferred ratio of micro particulate to dissolved dapsone is five or less.

In another embodiment, a composition comprises about 1% carbomer, about 80-90% water, about 10% ethoxydiglycol, about 0.2% methylparaben, about 0.3% to 3.0% dapsone including both micro particulate dapsone and dissolved dapsone, and about 2% caustic material. More particularly, the carbomer may include “CARBOPOL.RTM. 980” and the caustic material may include sodium hydroxide solution.

In a preferred embodiment, the composition comprises dapsone and ethoxydiglycol, which allows for an optimized ratio of micro particulate drug to dissolved drug. This ratio determines the amount of drug delivered, compared to the amount of drug retained in or above the stratum comeum to function in the supracomeum domain. The system of dapsone- and ethoxydiglycol may include purified water combined with “CARBOPOL.RTM.” gelling polymer, methylparaben, propylparaben, titanium dioxide, BHA, and a caustic material to neutralize the “CARBOPOL.RTM.”

Any of the carriers for the lytic enzyme may be manufactured by conventional means. However, if alcohol is used in the carrier, the enzyme should be in a micelle, liposome, or a “reverse” liposome, to prevent denaturing of the enzyme. Similarly, when the lytic enzyme is being placed in the carrier, and the carrier is, or has been heated, such placement should be made after the carrier has cooled somewhat, to avoid heat denaturation of the enzyme. In a preferred embodiment of the disclosure, the carrier is sterile.

The enzyme may be added to these substances in a liquid form or in a lyophilized state, whereupon it will be solubilized when it meets a liquid body.

The effective dosage rates or amounts of the lytic enzyme to treat the infection, and the duration of treatment will depend in part on the seriousness of the infection, the duration of exposure of the recipient to the infectious bacteria, the number of square centimeters of skin or tissue which are infected, the depth of the infection, the seriousness of the infection, and a variety of a number of other variables. The composition may be applied anywhere from once to several times a day, and may be applied for a short or long term period. The usage may last for days or weeks. Any dosage form employed should provide for a minimum number of units for a minimum amount of time. The concentration of the active units of enzyme believed to provide for an effective amount or dosage of enzyme may be in the range of about 100 units/ml to about 500,000 units/ml of composition, preferably in the range of about 1000 units/ml to about 100,000 units/ml, and most preferably from about 10,000 to 100,000 units/ml. The amount of active units per ml may, in some circumstances, be as high as 5-10 million units/ml, particularly when treating gangrenous or S. fasciae infections. The number of active units and the duration of time of exposure depends on the nature of the infection, and the amount of contact the carrier allows the lytic enzyme(s) to have. It is to be remembered that the enzyme works best when in a fluid environment. Hence, effectiveness of the enzyme(s) is in part related to the amount of moisture trapped by the carrier. In another preferred embodiment, a mild surfactant is present in an amount effective to potentiate the therapeutic effect of the lytic enzyme. Suitable mild surfactants include, inter alia, esters of polyoxyethylene sorbitan and fatty acids (Tween series), octylphenoxy polyethoxy ethanol (Triton-X series), n-Octyl-.beta.-D-glucopyranoside, n-Octyl-.beta.-D-thioglucopyranoside, n-Decyl-.beta.-D-glucopyranoside, n-Dodecyl-.beta.-D-glucopyranoside, and biologically occurring surfactants, e.g., fatty acids, glycerides, monoglycerides, deoxycholate and esters of deoxycholate.

In order to accelerate treatment of the infection, the therapeutic agent may further include at least one complementary agent which can also potentiate the bactericidal activity of the lytic enzyme. The complementary agent can be penicillin, synthetic penicillins bacitracin, methicillin, cephalosporin, polymyxin, cefaclor. Cefadroxil, cefamandole nafate, cefazolin, cefixime, cefinetazole, cefonioid, cefoperazone, ceforanide, cefotanine, cefotaxime, cefotetan, cefoxitin, cefpodoxime proxetil, celtazidime, ceftizoxime, ceftriaxone, cefriaxone moxalactam, cefuroxime, cephalexin, cephalosporin C, cephalosporin C sodium salt, cephalothin, cephalothin sodium salt, cephapirin, cephradine, cefuroximeaxetil, dihydratecephalothin, moxalactain, loracarbef, mafate, chelating agents, streptomycin, erythromycin, chloramphenicol, numerous other antibiotics, and any combinations thereof in amounts which are effective to synergistically enhance the therapeutic effect of the lytic enzyme. It should be noted that virtually any antibiotic may be used as a complementary agent with any of the recombinant lytic enzymes.

Additionally, the therapeutic agent may further comprise the enzyme lysostaphin for the treatment of any Staphylococcus aureus bacteria. Mucolytic peptides, such as lysostaphin, have been suggested to be efficacious in the treatment of S. aureus infections of humans (Schaffner et al., Yale J. Biol. & Med., 39:230 (1967) and bovine mastitis caused by S. aureus (Sears et al., J. Dairy Science, 71 (Suppl. 1): 244(1988)). Lysostaphin, a gene product of Staphylococcus simulans, exerts a bacteriostatic and bactericidal effect upon S. aureus by enzymatically degrading the polyglycine crosslinks of the cell wall (Browder et al., Res. Comm., 19: 393-400 (1965)). U.S. Pat. No. 3,278,378 describes fermentation methods for producing lysostaphin from culture media of S. staphylolyticus, later renamed S. simulans. Other methods for producing lysostaphin are further described in U.S. Pat. Nos. 3,398,056 and 3,594,284. The gene for lysostaphin has subsequently been cloned and sequenced (Recsei et al., Proc. Natl. Acad. Sci. USA, 84: 1127-1131 (1987)). The recombinant mucolytic bactericidal protein, such as r-lysostaphin, can potentially circumvent problems associated with current antibiotic therapy because of its targeted specificity, low toxicity and possible reduction of biologically active residues. Furthermore, lysostaphin is also active against non-dividing cells, while most antibiotics require actively dividing cells to mediate their effects (Dixon et al., Yale J. Biology and Medicine, 41: 62-68 (1968)). Lysostaphin, in combination with the lysin enzyme, can be used in the presence or absence of the listed antibiotics. There is a degree of added importance in using both lysostaphin and the lysin enzyme in the same therapeutic agent. Frequently, when a body has a bacterial infection, the infection by one genus of bacteria weakens the body or changes the bacterial flora of the body, allowing other potentially pathogenic bacteria to infect the body. One of the bacteria that sometimes co-infects a body is Staphylococcus aureus. Many strains of Staphylococcus aureus produce penicillinase, such that Staphylococcus, Streptococcus, and other gram positive bacterial strains will not be killed by standard antibiotics. Consequently, the use of the lysin and lysostaphin, possibly in combination with antibiotics, can serve as the most rapid and effective treatment of Staph infections. In yet another preferred embodiment, the disclosure may include mutanolysin, and lysozyme.

In preferred embodiments of the disclosure, the chimeric and/or shuffled lytic enzymes for Pseudomonas, Staphylococcus, Streptococcus, and the other dermatogically oriented bacteria, either individually or in combinations thereof, may be incorporated into the carrier, or into a bandage to be used on burn patients, or in a solution or cream carrier. These enzymes may be used in combination with holin proteins and other lytic enzymes. As with all uses of the lytic enzymes in the entire disclosure, holin proteins may also be used with the “unaltered” lytic enzyme(s) or with the chimeric and/or shuffled lytic enzymes.

These enzymes may find their greatest usefulness or effectiveness as a prophylactic, after surgery or after there has been a skin or tissue wound. Any wound dressing should include any or all of the enzymes and optionally the holins. Sprays and gels containing the enzymes and holin protein may be applied to a wound before stitches or bandages are applied. Similarly, bandages and time release bandages may include these enzymes.

For more serious dermatological and deep tissue infections, such as those of Peptostreptococcus and other anaerobic bacteria which can cause tissue necrosis, parenteral treatment of the bacteria will be necessary, as well as topical treatment.

Veneral Diseases

There are a few primary bacteria that cause bacterial venereal diseases.

Neisseria gonorrhoeae, a gram negative diplococcus, causes gonorrheal urethritis, also known as gonorrhea. In men, the infection can cause itching, and discomfort in the urethra, mainly resulting from restrictions in the urethra. In women, the infected fallopian tubes can scar, often resulting in sterility or in increases changes of ectopic pregnancies. Secondary infections can result.

Treponema palladium, a spirochete, causes syphilis. There are three very well known stages of syphilis. A chancre (red sore) appears in the first stage. In males, the chancre is normally on or near the penis or anus (depending on one's sexual orientation). In females, the chancre appears near the anus, on the vaginal wall, or cervix.

In the second stage of syphilis, rashes develop on the extremities, along with oral lesions in the mouth and on the lips, joint pain, fevers, and general illness. The first and second stages of syphilis occur in the first several weeks of infections.

The third or tertiary stage of syphilis occurs years later, often with no symptoms between the second and third stages of the illnesses. However, the tertiary stage is extremely dangerous. Lesions of the heart, liver, skin, bones and most of all the central nervous systems result. Congenital syphilis finally results, with lesions on the brain.

Another bacteria which may cause venereal disease or at least cause ulceration of the genitalia is Haemophilus ducreyi.

Venereal diseases are extremely serious. Thus rapid treatment and cure are essential. Any barrier of product that could be used prophylactically would reduce the spread of these diseases.

In women, N. gonorrhoeae can cause symptomatic urethritis, which is characterized by “internal” dysuria and pyuria. (See Harrison's 15^{th} Edition of “Principles of Internal Medicine,” page 840-841.

Today, both Treponema pallidum and Neisseria gonorrhoeae have grown increasingly resistant to most antibiotics. Every year, new strains that are more and more resistant to traditional antibiotics develop. In some cases, patients are now being given some of the strongest antibiotics currently available.

Neisseria gonorrhoeae has grown increasingly resistant to most antibiotics. Every year, new strains that are more and more resistant to traditional antibiotics develop. In some cases, patients are now being given some of the strongest antibiotics currently available.

The use of phage associated lytic enzymes to treat bacteria causing venereal disease will help stem and eliminate the disease. There are a number of different ways of administering the lytic enzyme, which may be, of course, in the form of a lytic enzyme, shuffled lytic enzyme, chimeric lytic enzyme, or combination thereof. While administration of the enzyme may be in the form of a pill, it is most advisable that the enzyme be administered parenterally. The enzyme may be administered intravenously or intramuscularly. In some cases where Treponema pallidum has remained untreated and reaches the most severe forms of the tertiary stage, it may be necessary to additionally administer the enzyme intrathecally.

However, for those cases in which intravenous and intramuscular treatment are appropriate, treatment may be done hypodermically (b)y means of a syringe) from one to several times a day, or treatment may be done on a continuous basis, 24 hours a day for several days until all signs of the bacteria have vanished. The administration of the enzymes on a continuous basis may be done by means of a drip feed in a hospital or care facility, or the patient may have a mechanical or electromechanical pump for administering the enzymes while the patient maintains his normal activities (excepting sexual activities). It is highly recommended that intrathecal administration of any drug only be done in a hospital or medical care facility.

The carrier for the enzyme(s) being administered parenterally may be comprised of the substances described above.

In cases where intramuscular injection is the chosen mode of administration, an isotonic formulation is preferably used again using the same substances described previously.

It is also important to the effective dosage rates or amounts of the lytic enzyme(s) to be administered parenterally, and the duration of treatment will depend in part on the seriousness of the infection, the weight of the patient, the duration of exposure of the recipient to the infectious bacteria, the number of square centimeters of skin or tissue which are infected, the depth of the infection, the seriousness of the infection, and a variety of a number of other variables. The composition may be applied anywhere from once to several times a day, and may be applied for a short term or long term period. The usage may last for days or weeks. Any dosage form employed should provide for a minimum number of units for a minimum amount of time. The concentration of the active units of enzyme believed to provide for an effective amount or dosage of enzyme may be in the range of about 100 units/ml to about 10,000,000 units/ml of composition, preferably in the range of about 1000 units/ml to about 10,000,000 units/mil, and most preferably from about 10,000 to 10,000,000 units/ml. The amount of active units per mi and the duration of time of exposure depends on the nature of infection, and the amount of contact the carrier allows the lytic enzyme to have. It is to be remembered that the enzyme works best when in a fluid environment. Hence, effectiveness of the enzyme is in part related to the amount of moisture trapped by the carrier.

In order to accelerate treatment of the infection(s), the therapeutic agent may further include at least one complementary agent which can also potentiate the bactericidal activity of the lytic enzyme. The complementary agent can be any antibiotic effective, or at least partially effective, against the venereal bacteria Similarly, other lytic enzymes may be included to treat other bacterial infections.

Additionally, as the venereal infections are concentrated in (but usually not limited to) the urinary tract, it is important to have a carrier, preferably liquid or even pill form, that will ensure that the bladder and urethra receive a relatively high concentration of the lytic enzyme for treating the infection.

Not all individuals practice abstinence while they have a venereal disease. Indeed, many individuals are often not even aware they are carrying a bacterial venereal disease. Consequently, to reduce the spread of the disease, and as a courtesy to the partner who may have the disease, the surface of condoms, in the lubrication coating, may be coated with the lytic enzymes for Neisseria gonorrhoeae and/or Treponema pallidum. This will help kill any bacteria in the vagina which the enzyme comes in contact with.

These lytic enzymes may also be incorporated into a tampon. Used properly, this will help to kill the appropriate venereal bacteria. The vagina has a great mucosal surface area. This area is a vast breeding ground for bacteria, and provide a great surface area -for harboring and spreading the disease. These enzymes may be used prophylactically to prevent the spread of bacterial venereal disease. Besides being incorporated into tampons and lubricants, the enzymes and optionally the holin protein may be included in vaginal lubricants, and diaphragm creams and lubricants. The enzymes and holin protein may be applied even to man's genitalia before sex. Besides containing the enzymes, the creams may include nonoxynol-9, and may be flavored.

Vaginal Infections

Yet another use for the lytic enzymes is for the prophylactic or therapeutic treatment of vaginal infections, other than those infections which are venereal in nature.

For example, bacterial vaginosis may be caused by Haemophilus vaginitis, G. vaginalis, Mycoplasma hominis, and several anaerobic bacteria (Mobiluncus spp. Prevotella spp. (Formerly Bacteroides spp.), and Peptostreptococcus spp.] More specifically, vaginosis may be caused by Gardnerella vaginalis, Prevotella spp., Mobiluncus spp. Peptostreptococci (including the species enumerated earlier), and genital mycoplasmas (see Braunwald et al.), Harrison's Principles of Internal Medicine, 2001, p. 1014).

Additionally, Enterobacter species infections may also cause gynecologic infections, including endometriosis and pelvic cellulites.

The treatment for these infections comprises treating the vaginal infection with an effective amount of at least one lytic enzyme produced by a bacteria being infected with a bacteriophage specific for that bacteria, wherein that lytic enzyme is incorporated in a carrier or mode of delivery to be placed in a vaginal canal The lytic enzyme(s) used to treat bacterial infections of the vagina may be either supplemented by chimeric and/or shuffled lytic enzymes, or may be itself a chimeric and/or shuffled lytic enzyme. Similarly, a holin protein may be included, which may also be a chimeric and/or shuffled lytic enzyme. The preferred carrier is a tampon, vaginal douche, or vaginal suppository. A pad may also be used as a carrier, although it is not as effective.

While any number of bacteria could be treated using this composition and method, it is believed that the most optimum use of this treatment composition and method would be for the treatment of an E. coli and Streptococcus B infection. Vaginal infections caused by Group B Streptococcus can cause neonatal meningitis resulting in brain damage and premature death of the infant. Lytic enzymes incorporated into a tampon specific for group B Streptococcus would eliminate the group B organisms without disturbing normal flora so that woman would not be overcome by possible yeast infections post antibiotic therapy. The use of these enzymes to treat or prevent group B streptococcus would also reduce or eliminate the risk of neonates getting infected. The use of the lytic enzymes in the vagina would provide a prophylactic effect, although therapeutic use would also be advisable.

To produce a pad or tampon containing the enzyme, the lytic enzymes can be applied in a solution to the tampon, and allowed to dry. The lytic enzyme may be incorporated into the pad or tampon by any other means known in the art, including lyophilization, spraying, etc. The tampons and pads may also be kept slightly moist, and in a sealed wrapper until ready for use. In that case, bacteriacide and bacteriastatic compounds and inhibitors should be present in the tampons and pads. The method to be used for incorporating the lytic enzyme into the tampon or pad can be one of die methods known in the art for incorporating a pharmaceutical product. In another embodiment of the disclosure, the lytic enzyme is incorporated into a vaginal suppository. The vaginal suppository into which the lytic enzyme is being incorporated may be a standard vaginal suppository, comprised of glyceride, alginate, starch, other standard binders and any combinations thereof. A vaginal suppository may also be used to treat or prevent venereal diseases.

When using a tampon as the carrier, it is best to insert the tampon in the vagina and leave it in for up to 12 hours to distribute the enzyme vaginally.

As with other lytic enzymes, it is preferable that the pH be kept in a range of about 4.0 and about 9.0 even more preferably at a pH range of between about 5.5 and about 7.5. As described above with the other lytic enzyme, the pH can be moderated by the use of a buffer. The buffer may contain a reducing agent, and more specifically dithiothreitol. The buffer may also contain a metal chelating reagent, such as ethylenediaminetetracetic disodium salt and the buffer may be a citrate-phosphate buffer. As with all compositions described in this patent, the composition may, further include a bacteriacidal or bacteriastatic agent as a preservative.

The lytic enzyme(s) are preferably present in a concentration of about 100 to about 500,000 active enzyme units per milliliter of fluid in the wet environment of the vaginal tract, preferably about 100 to about 100,000 active enzyme units per milliliter of fluid, and preferably present in a concentration of about 100 to about 10,000 active enzyme units per milliliter of fluid in the wet environment of the vaginal tract. However, greater concentrations of the enzyme up to 5,000,000 -10,000,000 units/ml, may be used.

Eye Infections

Another use of the disclosure is for the prophylactic and therapeutic treatment of eye infections.

There are a number of different bacteria which may cause infections of the eye. In a preferred embodiment of the disclosure, the bacteria being treated is/are Hemophilus, (in particular Haemophilus influenzae), and/or Staphylococcus. Haemophilus influenzae can be a major cause of “pink eye” or conjunctivitis. Additionally, Pseudomonas, in particular, can also cause eye infections.

Mycobacterium tuberculosis, Mycobacterium leprae, Treponema pallidum, and Francisella tularensis may infect the uvea, retina, vitreous and sclera.

Pseudomonas aeruginosa may cause corneal keratitis as a result of prolonged use of contact lenses.

Cryptococcus and Aspergillus can cause posterior uveitis.

Other agents which can cause eye infections are V. alginolyticus and Enterobacteriaceae.

The method of treatment comprises administering eye drops which comprise an effective amount of at least one lytic enzyme produced by the bacteria being infected with a bacteriophage specific for the bacteria and a carrier capable of being safely applied to an,eye, with the carrier containing the lytic enzyme. The eye drops are in the form of an isotonic solution. The pH of the solution should be adjusted so that there is no irritation of the eye, which in turn would lead to possible infection by other organisms, and possible damage to the eye. While the pH range should be in the same range as for other lytic enzymes, the most optimal pH will be in the range of from 6.0 to 7.5. Similarly, buffers of the sort described above for the other lytic enzymes should also be used. Other antibiotics which are suitable for use in eye drops may be added to the composition containing the lytic enzymes. Bactericides and bacteriostatic compounds may also be added. As stated above, this lytic enzyme may be either supplemented by chimeric and/or shuffled lytic enzymes, or may be itself a chimeric and/or shuffled lytic enzyme. Similarly, a holin protein may be included which may also be a chimeric and/or shuffled lytic enzyme.

Additionally, a contact leans cleaning solution or an overnight lens solution may include any or all of these lytic enzymes.

It is to be remembered that all of the enzymes can be used for prophylactic and therapeutic treatments of the bacteria for which the enzymes are specific.

Additionally, a carrier may have more than one lytic enzyme. For instance, a throat lozenge may comprise just a lysin enzyme (which lyses the Streptococcus A strain causing “strep” throat) or it may also include the lytic enzymes for Hemophilosis. Similarly, the carrier for treating burns and wounds, or infections of the skin, may contain just one lytic enzyme, or a combination of lytic enzymes, for the treatment of Pseudomonas, Streptococcus, Staphylococcus, or any other of a number of bacteria. The carrier may include any combination of lytic enzymes, shuffled lytic enzymes, chimeric lytic enzymes, and holin proteins.

Non-human animals can also suffer from these same bacterial diseases of the eye, and may be treated according.

Additionally, other bacteria can cause eye infections. For instance, Mycoplasma felis can cause conjunctivitis in cats.

Bacterial Dental Infections

Lytic enzymes can also be used to fight dental caries. For example, a lytic enzyme specific for Streptococcus mutans may be incorporated in a toothpaste or oral wash. Similarly, this lytic enzyme may also be incorporated into a chewing gum or lozenge. Any other carrier can be used that allows for the exposure of the mouth, gums, and teeth to the lytic enzyme. Other target cariogenic or periodonto-pathogenic bacteria which may be treated come from the genera Actinobacillus, Actinomyces, Bacteroides, Capnocytophaga, Eikenella, Eubacterium, Fusobacterium, Haemophilus, Lactobacillus, Peptostreptococcus, Porphyromonas, Prevotella, Rothia, Selenomonas, Streptococcus, Treponema, and Wolinella. More specific gram positive, caries-related species include but are not limited to: Actinomyces viscosus, A. naeslundii, and Streptococcus mutants, S. sobrinus, aid Lactobacillus casei.

The lytic enzyme may also be incorporated in a lyophilized or dried form in tooth powder. If the lytic enzyme is to be used in an oral wash, it is preferred that the oral wash not contain any alcohol, so as to not denature the enzyme. The enzyme can also be in a liposome when nixed in with the toothpaste or oral wash. The concentrations of the enzyme units per ml of toothpaste, oral wash, chewing gum, candy or lozenge can be in the range of from about 100 units/ml to about 500,000 units/ml of composition, preferably in the range of about 1000 units/ml to about 100,000 units/ml, and most preferably from about 10,000 to 100,000 units/ml. In some circumstances, the amounts of enzyme may range up to over 1,000,000 units/ml, and possibly much higher. The pH of the toothpaste or oral wash should be in a range that allows for the optimum performance of the enzyme, while not causing any discomfort to the user of the toothpaste or oral wash. Again, as with the other uses of lytic enzymes, the lytic enzyme used to treat dental caries may be either supplemented by chimeric and/or shuffled lytic enzymes, or may be itself a chimeric and/or shuffled lytic enzyme. Similarly, a holin protein may be included, which may also be a chimeric and/or shuffled lytic enzyme. The toothpastes, lozenges, gums, mouth wash, candy, and toothpowders may include any of their normal substances, as long as they do not interfere with the actions and viability of the enzyme(s). Similarly, there may be as many different specific enzymes as desired. In a candy, it is preferred that an artificial sweetener be used, although this is not always necessary.

Otitis Bacterial Infections

A number of different bacteria are responsible for bacterial infections of the ear. Most ear infections occur in early childhood. The bacteria most likely to cause these childhood ear infections are Streptococcus pneumoniae, H. influenzae and B-hemolytic Streptococci. Additionally, E. coli, as well as Klebsiella, may also infect the middle ear, mastoids and/or paranasal sinuses.

Chronic infections may be caused by V. alginolyticus, Pseudomonas aeruginosa and Streptococcus aureus which, if unchecked, can cause permanent damage as a result of destructive necrotizing otitis media. Untreated, the chronic infection has the potential to perforate the eardrum, encroaching on the ossicles or labyrinth, spreading into the mastoid spaces and even penetrating into the cranial vault to produce a temporal cerebritis or abscess ( p. 767, Cotran, et al, Robbins Pathologic Basis of Disease, 6^{th} edition, 1999).

The unaltered, shuffled and/or chimeric lytic enzyme (along with the holin protein), may be applied to an infected ear by delivering the enzyme(s) in an appropriate carrier to the canal of the ear. The carrier may comprise sterile aqueous or oily solutions or suspensions. An isotonic solution may be used. The lytic enzyme(s) may be added to the carrier, which may also contain suitable preservatives, and preferably a surface active agent. Bactericidal and fungicidal agents preferably included in the drops are phenylmercuric nitrate or acetate (0.002%), benzalk-onium chloride (0.01%) and chlorhexidine acetate (0.01%). Suitable solvents for the preparation of an oily solution include glycerol, diluted alcohol and propylene glycol. Additionally, any number of other ear drop carriers may be used.

The concentrations and preservatives used for the treatment of otitis media and other similar ear infections are the same as discussed for eye infections, and the carrier into which the enzyme goes is similar or identical to the carriers for treatment of eye infections. Additionally, the carrier may typically include vitamins, minerals, carbohydrates, sugars, amino acids, proteinaceous materials, fatty acids, phospholipids, antioxidants, phenolic compounds, isotonic solutions, oil based solutions, oil based suspensions, and combinations thereof.

As with the other treatments, various phage associated lytic enzymes specific for the specific bacteria may be used, and more than one bacteria may be treated at a time.

General Illnesses

A number of the bacteria discussed above, as well as other bacteria, cause a number of illnesses which may best be treated by parenteral use of specific phage associated lytic enzymes. H. influenzae can cause septicemia, endocarditis and suppurative arthritis. These illnesses can be treated by the parenteral use of a phage associated lytic enzyme specific for H. influenzae.

Similarly, H. influenzae can also cause meningitis. In such cases, there should be an intrathecal administration of the lytic enzymes in all of their forms, as well as the holin proteins. It should be noted that Klebsiella may also cause both neonatal and surgically related meningitis.

Neisseria meningitis causes cerebrospinal meningitis and bacteremia, which can lead to intravascular coagulation.

E. coli (Escherichia coli), probably one of the most prevalent bacteria in existence, and is normally found in the large intestine, can, in its pathogenic form, be one of the more dangerous forms of bacteria known to man.

Among the many problems or illnesses that E. coli may cause are bacteremia, biliary infection, brain abscesses (normally due to ear infections), diarrhea, emphysematous cholecystitis (inflammation of the gall bladder and bile ducts), fever, hemolysis, hepatic abscesses, neonatal infection, peritoneal infection, proctocolitis, urethritis, and urinary tract infections.

Bacteremia is one of the most serious manifestations of E. coli infections. It is most common in patients with urinary tract infection and biliary or intraperitoneal sepsis, and can cause septic shock. (See Harrisons Principles of Internal Medicine, 12^{th} edition, p. 600).

E. coli also accounts for 75% of urinary tract infections. Urethritis and cystitis are the usual forms of the infection.

K. pneumoniae is second only to E. coli as a urinary tract pathogen. Klebsiella infections are encountered far more often now than in the past. This is probably due to the bacterium's antibiotic resistance properties.

Bacteremia may also be caused by Klebsiella, with infections of the urinary tract, respiratory tract and abdomen each accounting for 15-30% of Klebsiella bacteremias (Hanisons, 15^{th} Edition, p. 957).

Bacteria that belong to the family Enterobacteriaceae include Escherichia, Klebsiella, Proteus, Enterobacter, Serratia, Citrobacter, Morganella, Providencia and Edwardsiella) as well as die genus Acinetobacter from the family Neisseriaceae. Theoretically, these gram negative bacteria can infect almost every organ of the body.

Proteus mirabilis often infects those in long term care facilities, hospitals, schools, and other public facilities. P. mirabilis infections may result in bladder infections and septicemia, which includes the symptoms of fever, dyspnea, acute confusion, chills, tachycardia, bacteremia, headache, and tachypnea.

Enterobacter infections, as noted above, can infect many parts of the body, causing many illnesses. The most common of the Enterobacter bacteria are E. cloacae and E. aerogenes, with E. agglomerans, E. sakazakii and E. gergoviae appearing less frequently.

Enterobacter often infect substantially compromised patients such as the very young, the very old, and, especially, those with severe underlying disease, such as neoplasms, or immune suppression including human immunodeficiency virus infection, usually in the later stages of the disease. (See J Chemother April 2001;13(2):195-201)

Enterobacter sakazakii may cause neonatal meningitis/sepsis through contaminated formula.

Enterobacter cloacae is often associated with infections relating to dialysis. However, Enterobacter cloacae may also cause intra-abdominal infections, and urinary tract infections.

Bacteria from the genus Acinetobacter are ubiquitous in nature and can be found in water, soil, and living organisms. (Ref: http://arch.rivm.nl/enemti/Tle%20genus%20Acin). There are a number of species of Acizetobacter, including A. calcoaceticus and Actiyobacter genospecies 3 and 13TU; however, A. baumanni is responsible for the majority of Acinetobacter infections. A. baumanni, can cause infections of the skin and the respiratory and gastrointestinal tracts.

The various species of Acinetobacter may cause bacteremia, urinary tract infections, and secondary meningitis. As noted above, Acinetobacter may also cause serious infections in burn patients, and nosocomial pneumonia.

Serratia marcesans, particularly in institutions and as a result of dialysis, can infect the genitourinary tract. Additionally, Serratia marcesans also infects surgical sites.

Another member of the Enterobacteriaceae family is a genus of bacteria known as Citrobacter, with C. freundii and C. koseri causing the majority of human Citrobacter infections. Citrobacter bacteria are very similar to Enterobacter and Acinetobacter, epidemiologically and clinically speaking. While Citrobacter may be found naturally in the intestinal tracts of some animals and is part of the normal fecal flora in a minority of healthy humans, the bacteria does tend to infect immunocompromised individuals. Morganella and Providencia organisms and in particular M. morganii and P. rettgeri are primarily urinary tract infections, and are most frequently associated with primarily long term catheterization. Long tern infection by these two organisms can lead to kidney stones.

P. aeruginosa infections are not limited to the respiratory tract, but in fact can also be the cause of bloodstream infections in immunocompromised patients. Citing Harrison's Principles of Internal Medicine, 15th edition, page 964, “[t]he clinical features of P. aeruginosa bacteremia are similar to those of other forms of bacteremia. Common primary sites of infection include the urinary tract, gastrointestinal tract, lungs, skin and soft tissues, and intravascular foci, including indwelling centrol venous catheters. Fever tachypnea, tachycardia, and prostration are common. Disorientation, confusion or obtundation may be evident. Hypertension can progress to refractory shock. Renal failure, adult respiratory distress syndrome, and disseminated intravascular coagulation occur as complications.”

P. aeruginosa may also cause endocarditis injection. This bacteria can also cause, or can be responsible for, meningitis and brain abscesses. B. cepacia is a bacteria “related” to Pseudomonas found in hospital epidemics, which can cause urinary tract infections, meningitis, peritonitis, bacteremia, and endocarditis.

As noted above, Burkholderia pseudomallei, a bacteria similar to Pseudomonas which is found primarily in Southeast Asia, can cause meliodosis; a disease that may resemble bronchitis and pneumonia, but can also have the symptoms of tuberculosis. However, meliodosis can also cause or result in septicemia, arthritis, meningitis, and enlargement of the liver and spleen. Other symptoms may include meningitis, arthritis, diarrhea, and pustular lesions over much of the body. This bacteria may also infect horses, cattle, sheep, goats, pigs, dogs, cats and rodents.

Pseudomonas mallei (sometimes referred to as Malleomyces mallei) can be transmitted to humans by means of eating infected horse meat. Septicemia, lesions in the nasal cavity, infections in the lungs accompanied by the formation of nodules, are all symptoms of this infection that can lead to death.

Perhaps one of the most well known, and in some ways most terrifying bacteria, are those found in the Clostridia genus.

Clostridium tetani causes tetanus, a neurological disorder which can cause muscle paralysis and spasms. Found in many different environments, tetanus is easily preventable by vaccination, yet is still found around the world. There are also several different forms of the diseases caused by tetanus, including neonate tetanus, although the elderly are most at risk for developing the severest form of tetani. Most forms of tetanus are a result of simple wound or puncture injuries, often while the individual is working or playing in soil. As C. tetani is a spore, toxin production does not occur until the spore germinates. Generalized tetanus is the most common form of the disease, although a less common form of the disease, known as local tetanus, affects only those muscles near the wound.

Clostridium botulinum can cause a potentially fatal paralytic disease. While botulism can be spread by contaminated food, it can also be obtained by a wound.

Clostridia are frequently the cause of deep tissue infections. Some of the signs of Clostridia infection are frostbite with gas gangrene, infection of a stump in an amputee, pelvic abscesses, subcutaneous abscesses, intra abdominal sepsis, perianal abscesses, infection of a renal cell carcinoma, infection of an aortic graft, etc. In cases of intra abdominal sepsis, (often related to an intestinal perforation), C. ramosum, C. petfringes, and C. bifermentans are commonly isolated.

Clostridia wound infections may also lead to Clostridial myonecrosis, also known as gas gangrene. The nature of the wound needs to be deep, and necrotic, without communication with the surface, thereby providing anaerobic conditions. Most cases are caused by C. perfringens, and also by C. novyi, C. septicum, and C. histolyticum. (See Harrison's).

The Clostridia genus can also cause bacteremia and sepsis.

Borrelia burgdorferi, Borrelia recurrentis, and Borrelia spirochetes can all cause or contribute to Lyme disease, a multi organ disease with a variety of illnesses, often beginning with and causing a chronic arthritis. In some cases, encephalitis may result. Borrelia spirochetes may also cause relapsing fever.

For cases in which the bacteria are infecting deep tissues, sexual organs, systemic infections, and other internal organs (including the large intestine, skin infections, and nasopharynx infections, where there are other modes of administration for delivering the enzyme(s)), the lytic enzymes may be administered parenterally. More specifically, the lytic enzyme, holin protein, chimeric enzyme, shuffled enzyme, and combinations thereof may be administered: intravenously, intramuscularly, subcutaneously, intrathecally, and subdermally. Intrathecal use would be most beneficial for treatment of bacterial meningitis, which would, for the purposes of this patent, include bacteria found in the spine and in the spinal fluid.

Additionally, bone and joint infections may be caused by anaerobic bacteria including Peptostreptococcus spp., Bacteroides spp., Fusobacterium spp. and Clostridium spp. These bacteria are usually found in the soft tissue near the bone. Prevotella and Porphyromonas spp. are found in the maxilla and mandible. (Harrison's Principles of Internal Medicine, 15^{th} Edition, 2001, p. 1014). These bacteria may be treated parenterally.

The composition may be used for the (parenteral) therapeutic treatment of Pseudomonas, Clostridium, Staphylococcus, Proteus, Burkholderia, Morganella and Providencia, Treponema, and Neisseria infections, among others listed above.

More than one lytic enzyme may be introduced into the infected body at a time.

The parenteral carrier may be comprised of distilled water, a saline solution, albumin, a serum, or any combinations thereof. More specifically, solutions for infusion or injection may be prepared in a conventional manner, e.g. with the addition of preservatives such as p-hydroxybenzoates or stabilizers such as alkali metal salts of ethylene-diamine tetraacetic acid, which may then be transferred into fusion vessels, injection vials or ampules. Alternatively, the compound for injection may be lyophilized either with or without the other ingredients and be solubilized in a buffered solution or distilled water, as appropriate, at the time of use. Non-aqueous vehicles such as fixed oils, liposomes, and ethyl oleate are also useful herein.

An isotonic formulation is preferably used for the parenteral carrier(s). Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation. The pharmaceutical preparations according to the present disclosure are provided sterile and pyrogen free.

The carrier suitably contains minor amounts of additives such as substances that enhance isotonicity and chemical stability. Such materials are nontoxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulin; hydrophilic polymers such as polyvinylpyrrolidone; glycine; amino acids such as glutamic acid, aspartic acid, histidine, or arginine; monosaccharides, disaccharides, and other carbohydrates including cellulose or its derivatives, glucose, mannose, trehalose, or dextrins; chelating agents such as EDTA; sugar alcohols such as mannitol or sorbitol; counter-ions such as sodium; non-ionic surfactants such as polysorbates, poloxamers, or polyethylene glycol (PEG); and/or neutral salts, e.g., NaCl, KCl, MgCl.sub.2, CaCl.sub.2, etc.

Glycerin or glycerol (1,2,3-propanetriol) is commercially available for pharmaceutical use. It may be diluted in sterile water for injection, or sodium chloride injection, or other pharmaceutically acceptable aqueous injection fluid, and used in concentrations of from about 0.1 to 100% (v/v), preferably about 1.0 to about 50% more preferably about 20%.

DMSO is an aprotic solvent with a remarkable ability to enhance penetration of many locally applied drugs. DMSO may be diluted in sterile water for injection, or sodium chloride injection, or other pharmaceutically acceptable aqueous injection fluid, and used in concentrations of from about 0.1 to 100% (v/v).

The carrier vehicle May also include Ringer's solution, a buffered solution, and dextrose solution, particularly when an intravenous solution is prepared.

These solutions may be used for any of the enzymes mentioned herein. Prior to, or at the time the lytic enzyme is put in the carrier system or oral delivery mode, it is preferred that the enzyme be in a stabilizing buffer environment for maintaining a pH range between about 4.0 and about 9.0, more preferably between about 5.5 and about 7.5 and most preferably at about 6.1. This pH range is most suitable for the lysin enzyme for Streptococcus.

The stabilizing buffer should allow for the optimum activity of the lysin enzyme. The buffer may be a reducing reagent, such as dithiothreitol. The stabilizing buffer may also be or include a metal chelating reagent, such as ethylenediaminetetracetic acid disodium salt, or it may also contain a phosphate or citrate-phosphate buffer. The buffers found in the carrier can serve to stabilize the environment for the lytic enzymes.

The effective dosage rates or amounts of the chimeric and/or shuffled lytic enzymes to treat the infection, and the duration of treatment will depend in part on the seriousness of the infection, the duration of exposure of the recipient to the infectious bacteria, the number of square centimeters of skin or tissue which are infected, the depth of the infection, the seriousness of the infection, and a variety of a number of other variables. The composition may be applied anywhere from once to several times a day, and may be applied for a short or long term period. The usage may last for days or weeks. Any dosage form employed should provide for a minimum number of units for a minimum amount of time.

The concentration of the active units of enzyme believed to provide for an effective amount or dosage of enzyme may be in the range of about 100 units/ml to about 500,000 units/ml of composition, preferably in the range of about 1000 units/ml to about 100,000 units/ml, and most preferably from about 10,000 to 100,000 units/ml. The amount of active units per ml and the duration of time of exposure depends on the nature of infection, and the amount of contact the carrier allows the lytic enzyme to have. It is to be remembered that the enzyme works best when in a fluid environment. Hence, effectiveness of the enzyme is in part related to the amount of moisture trapped by the carrier. For the treatment of septicemia, and similar type general infections, there should be a continuous intravenous flow of therapeutic agent into the blood stream. The concentration of lytic enzymes for the treatment of septicemia is dependent upon the seriousness of the infection.

In some situations, it may be necessary to have the concentration as high as 5-10 million active units/ml.

When the lytic enzyme(s) need to be given parenterally, it may be possible to administer the enzymes in a mechanical or electromechanical pump, so that the patient does not have to stay in the hospital. In some instances, as the amount of therapeutic agent containing the active agent runs out, the patient can change the fluid reservoirs or tubules that bold the agent enzyme. The nature, extent, and seriousness of the infection, and the general health and lifestyle of the person, will generally be the determining factors as to whether the infected individual is a good candidate for an outpatient administration of the enzyme(s).

In order to accelerate treatment of the infection, the therapeutic agent may further include at least one complementary agent which can also potentiate the bactericidal activity of the lytic enzyme. The complementary agent can be penicillin, synthetic penicillins bacitracin, methicillin, cephalosporin, polymyxin, cefaclor, Cefadroxil, cefamandole nafate, cefazolin, cefixime, cefmetazole, cefonioid, cefoperazone, ceforanide, cefotanme, cefotaxime, cefotetan, cefoxitin, cefpodoxime proxetil, ceftazidime, ceftizoxime, ceftriaxone, cefriaxone moxalactam, cefuroxime, cephalexin, cephalosporin C, cephalosporin C sodium salt, cephalotlin, cephalothin sodium salt, cephapirin, cephradine, cefuroximeaxetil, dihydratecephalothin, moxalactam, loracarbef, mafate, chelating agents, streptomycin, erythromycin, chloramphenicol, numerous other antibiotics, and any combinations thereof in amounts which are effective to synergistically enhance the therapeutic effect of the lytic enzyme(s). As previously noted, virtually any antibiotic may be used with the various lytic enzymes, which include the shuffled and/or chimeric lytic enzymes, the holin proteins, etc.

Additionally, the therapeutic agent may further comprise the enzyme lysostaphin for the treatment of any Staphylococcus aureus bacteria. In yet another preferred embodiment, the disclosure may include mutanolysin, and lysozyme.

It is also advantageous to use these enzymes prophylactically. Whenever an individual undergoes surgery, it may be advisable to put any and all enzymes for bacteria that cause post-surgical infections in the iv drip. If neurosurgery has been performed, it may be helpful to have an intrathecal administration of the enzyme(s) for the bacteria that cause meningitis.

Similarly, administration of the enzymes may be given by all appropriate delivery and carrier means when there is, or has been, exposure or potential exposure to the pathogenic bacteria. Intravenous, muscular, and subdermal administration of the enzymes in all of their forms and the holin protein may be advantageous.

When a patient is undergoing hemodialysis, it is recommended that the appropriate lytic enzymes and proteins be added to the blood during the dialysis process.

Similarly, for some of the other bacteria, an enteric coated pill may be taken to avoid the gastrointestinal and urinary infections that may result.

Dermatological infections or exposure where there has not been a cut or wound may be dealt with by washing with a cleaning solution that contains the lytic enzyme.

Enzyme Delivery

It is expected that the enzymes will only have to be in the body a short time before they destroy the targeted bacteria. However, it may be necessary to make certain modifications to the bacteria, or to put the bacteria in a protected environment, to aid in their delivery and destruction of the bacteria.

In one preferred embodiment of the disclosure, the enzyme may be pegylated. For example, one or more activated poly(ethylene glycol) (PEG) derivatives, preferably from Shearwater Polymers, Inc., is attached to the enzyme. More specifically, PEG is a neutral, water-soluble, non-toxic polymer. The lack of toxicity from pegylation is reflected in the fact that PEG is one of the few synthetic polymers approved for internal use by the FDA, appearing in food, cosmetics, personal care products and pharmaceuticals. The true nature of PEG, however, is revealed by its behavior when dissolved in water. In an aqueous

By using PEGs, there is reduced immunogenicity and proteolysis. Carbohydrate and peptide receptor clearance mechanisms are “fooled” by PEG's “cloaking” ability. Less frequent dosing is required due to greatly increased body residence time. There is also improved efficacy due to increased concentration and longer dwell time at the site of action.

Additionally, the enzymes can be put in micelles, inverted micelles and liposomes for delivery and distribution to the appropriate sites, and to provide increased protection for the enzyme.

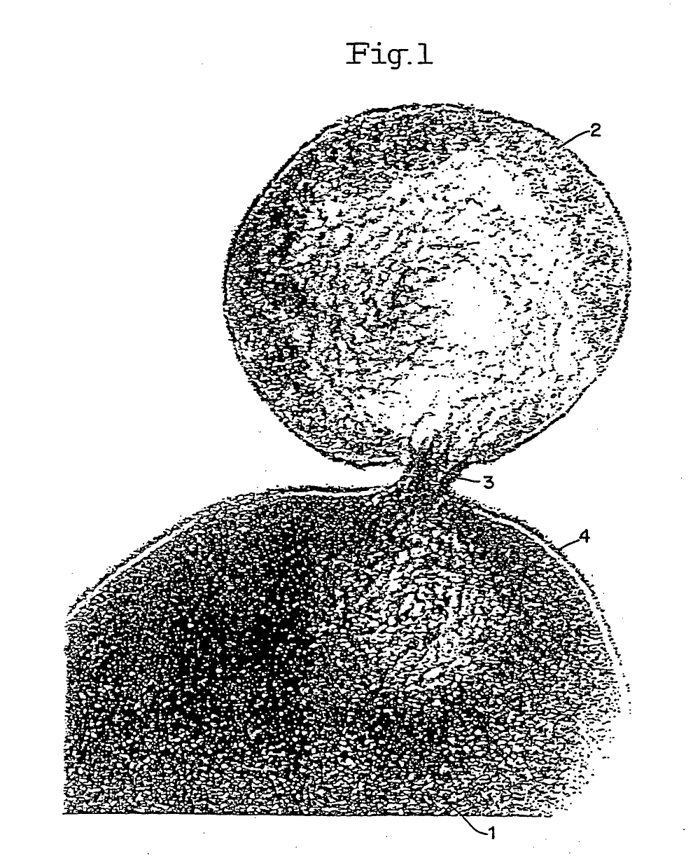

The use of lytic enzymes, including but not limited to holin lytic enzymes, chimeric lytic enzymes, shuffled lytic enzymes, and combinations thereof, rapidly lyse the bacterial cell. The thin section electron micrograph of FIG. 1 shows the results of a group A streptococci 1 treated for 15 seconds with lysin. The micrograph (25,000× magnification) shows the cell contents 2 pouring out through a hole 3 created in the cell wall 4 by the lysin enzyme.

As noted above, the use of the holin lytic enzyme, the chimeric lytic enzyme, and/or the shuffled lytic enzyme, may be accompanied by the use of a “natural” lytic enzyme, which has not been modified by the methods cited in U.S. Pat. No. 6,132,970, or by similar state of the art methods. Similarly, the natural proteins or lytic enzyme may be used without the chimeric or shuffled lytic enzymes. The phage associated lytic enzyme may be prepared as shown in the following example:

### EXAMPLE 1

Harvesting Phage Associated Lytic Enzyme

Group C streptococcal strain 26RP66 (ATCC #21597) or any other group C streptococcal strain is grown in Todd Hewitt medium at 37 degrees C. to an OD of 0.23 at 650 nm in an 18 mm tube. Group C bacteriophage (C1) (ATCC #21597-B1) at a titer of 5,000,000 is added at a ratio of 1 part phage to 4 parts cells. The mixture is allowed to remain at 37 degrees C. for 18 min at which time the infected cells are poured over ice cubes to reduce the temperature of the solution to below 15 degrees C. The infected cells are then harvested in a refrigerated centrifuge and suspended in 1/300th of the original volume in 0.1M phosphate buffer, pH 6.1 containing 5 mm dithiothreitol and 10 ug of DNAase. The cells will lyse releasing phage and the lysin enzyme. After centrifugation at 100,000 g for 5 hrs to remove most of the cell debris and phage, the enzyme solution is aliquoted and tested for its ability to lyse Group A Streptococci.

The number of units/ml in a lot of enzyme is determined to; be the reciprocal of the highest dilution of enzyme required to reduce the OD650 of a suspension of group A streptococci at an OD of 0.3 to 0.15 in 15 minutes. In a typical preparation of enzyme 400,000 to 4,000,000 units are produced in a single 12 liter batch.

Use of the enzyme in an immunodiagnostic assay requires a minimum number of units of lysin enzyme per test depending on the incubation times required. The enzyme is diluted in a stabilizing buffer maintaining the appropriate conditions for stability and maximum enzymatic activity, inhibiting nonspecific reactions, and in some configurations contains specific antibodies to the Group A carbohydrate. The preferred embodiment is to use a lyophilized reagent which can be reconstituted with water. The stabilizing buffer can comprise a reducing reagent, which can be dithiothreitol in a concentration from 0.001M to 1.0M, preferably 0.005M. The stabilizing buffer can comprise one or more immunoglobulin or immunoglobulin fragments in a concentration of 0.001 percent to 10 percent, preferably 0.1 percent. The stabilizing buffer can comprise a citrate-phosphate buffer in a concentration from 0.001M to 1.0M, preferably 0.05M. The stabilizing buffer can have a pH value in the range from 5.0 to 9.0. The stabilizing buffer can comprise a bacteriacidal or bacteriostatic reagent as a preservative. Such preservative can be sodium azide in a concentration from 0.001 percent to 0.1 percent, preferably 0.02 percent.

The preparation of phage stocks for lysin production is the same procedure described above for the infection of group C streptococcus by phage in the preparation of the lysin enzyme. However, instead of pouring the infected cells over ice, incubation at 37 degrees C. is continued for a total of 1 hour to allow lysis and release of the phage and the enzyme in the total volume. In order for the phage to be used for subsequent lysin production the residual enzyme must be inactivated or removed to prevent lysis from without of the group C cells rather than phage infection.

The use of chimeric or shuffled enzymes shows a great improvement as to the properties of the enzyme, as illustrated by the following examples:

### EXAMPLE 2

A number of chimeric lytic enzymes have been produced and studied. Gene E-L, a chimeric lysis constructed from bacteriophages phi X174 and MS2 lysis proteins E and L, respectively, was subjected to internal deletions to create a series of new E-L clones with altered lysis or killing properties. The lytic activities of the parental genes E, L, E-L, and the internal truncated forms of E-L were investigated in this study to characterize the different lysis mechanism based on differences in the architecture of the different membranes spanning domains. Electron microscopy and release of marker enzymes for the cytoplasmic and periplasmic spaces revealed that two different lysis mechanisms can be distinguished depending on penetration of the proteins of either the inner membrane or the inner and outer membranes of the E. coli. FEMS Microbiol. Lett. Jul. 1, 1998, 164(1); 159-67.

Also, an active chimeric cell wall lytic enzyme (TSL) is constructed by fusing the region coding for the terminal half of the lactococcal phage Tuc2009 lysin and the region coding for the C-terminal domain of the major pneumococcal autolysin. The chimeric enzyme exhibited a glycosidase activity capable of hydrolysing choline-containing pneumoccal cell walls.

### EXAMPLE 3

Isolation of the Pal Lytic Enzyme:

Recombinant E. coli DH5 (PMSP11) containing the pal lytic enzyme gene were grown overnight, induced with lactose, pelleted, resuspended in phosphate buffer, and broken by sonication. After centrifugation, the Pal enzyme in the supernatant was purified in a single step using a DEAE-cellulose column with elution by choline. Protein content was analyzed with the Bradford method. Using this method, a single protein band was identified by SDS-PAGE.

### EXAMPLE 4

Killing Assay:

S. pneumoniae of various serotypes and 8 different viridans streptococi were grown overnight and for most assays diluted and re-grown for 6 h to log phase of growth, pelleted and resuspended in 0.9% saline to an OD @ 620 nm of 1.0. In some experiments, stationary phase organisms were used. Killing assays were performed by adding 100, 1,000 or 10,000 U/mL of Pal to an equal volume of the bacterial suspension and incubating for 15 minutes at 37 C. Phosphate buffer alone served as control in place of enzyme. Bacterial counts before and after Pal or control phosphate buffer treatment were assessed by serial 10-fold dilutions at various time points and plated to determine colony forming units.

One unit (U) of Pal was defined as the highest dilution at which Pal decreased the OD of a pneumococcal strain by half in 15 minutes.

### EXAMPLE 5

Production of Chimeric Lytic Enzymes

A number of chimeric lytic enzymes have been produced and studied. Gene E-L, a chimeric lysis constructed from bacteriophages phi X174 and MS2 lysis proteins E and L, respectively, was subjected to internal deletions to create a series of new E-L clones with altered lysis or killing properties. The lytic activities of the parental genes E, L, E-L, and the internal truncated forms of E-L were investigated in this study to characterize the different lysis mechanism, based on differences in the architecture of the different membranes spanning domains. Electron microscopy and release of marker enzymes for the cytoplasmic and periplasmic spaces revealed that two different lysis mechanisms can be distinguished depending on penetrating of the proteins of either the inner membrane or the inner and outer membranes of the E. coli. FEMS Microbiol. Lett. Jul. 1, 1998, 164(1); 159-67.

Also, an active chimeric cell wall lytic enzyme (TSL) is constructed by fusing the region coding for the N-terminal half of the lactococcal phage Tuc2009 lysin and the region coding for the C-terminal domain of the major pneumococcal autolysin. The chimeric enzyme exhibited a glycosidase activity capable of hydrolysing choline-containing pneumoccal cell walls.

### EXAMPLE 6

Isolation of the Pal Lytic Enzyme

Recombinant E. coli DH5 (pMSP11) containing the pal lytic enzyme gene were grown overnight, induced with lactose, pelleted, resuspended in phosphate buffer, and broken by sonication. After centrifugation, the Pal enzyme in the supernatant was purified in a single step using a DEAE-cellulose column and elution with choline. Protein content was analyzed with the Bradford method. Using this method, a single protein band was identified by SDS-PAGE.

### EXAMPLE7

Killing Assay

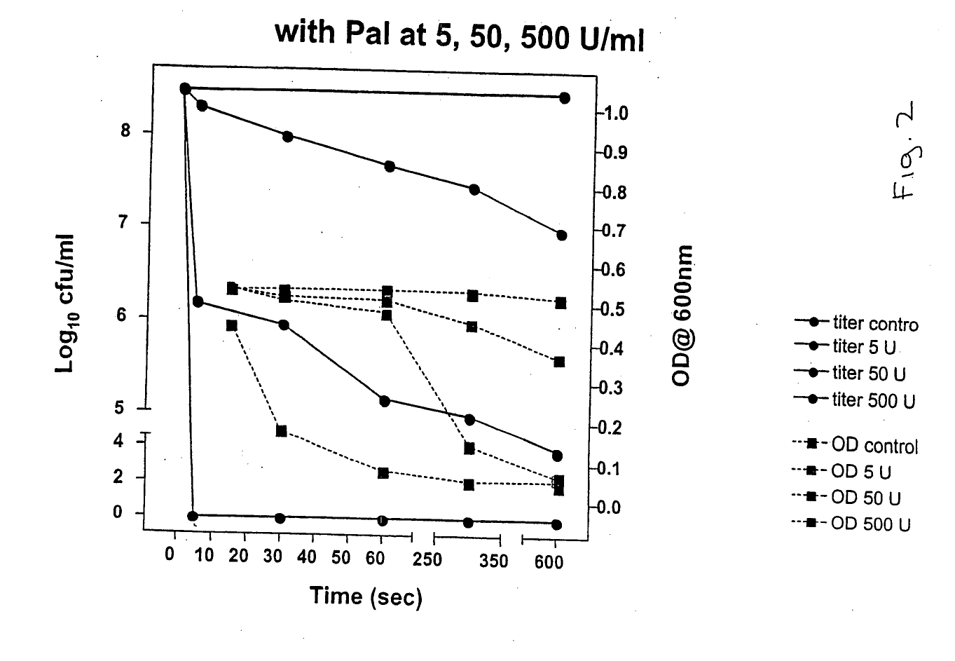

S. pneumoniae of various serotypes and 8 different viridans streptococci were grown overnight and for most assays diluted and re-grown for 6 h to log phase of growth, pelleted and resuspended in 0.9% saline to an OD @ 620 nm of 1.0. In some experiments, stationary phase organisms were used. Killing assays were performed by adding 100, 1,000 or 10,000 U/mL of Pal to an equal volume of the bacterial suspension and incubating for 15 minutes at 37 C. Phosphate buffer served as control in place of enzyme. Bacterial counts before and after Pal or control phosphate buffer treatment were assessed by serial 10-fold dilutions at various time points and plated to determine colony forming units. One unit (U) of Pal was defined as the highest dilution at which Pal decreased the OD of a pneumococcal strain by half in 15 minutes. The results, (see FIG. 2) show that the viability of Pneumococci dropped more than 8 logs in five seconds after adding the Pal enzyme.

### EXAMPLE 8

Susceptibility of Oral Streptococci to Pal Enzyme

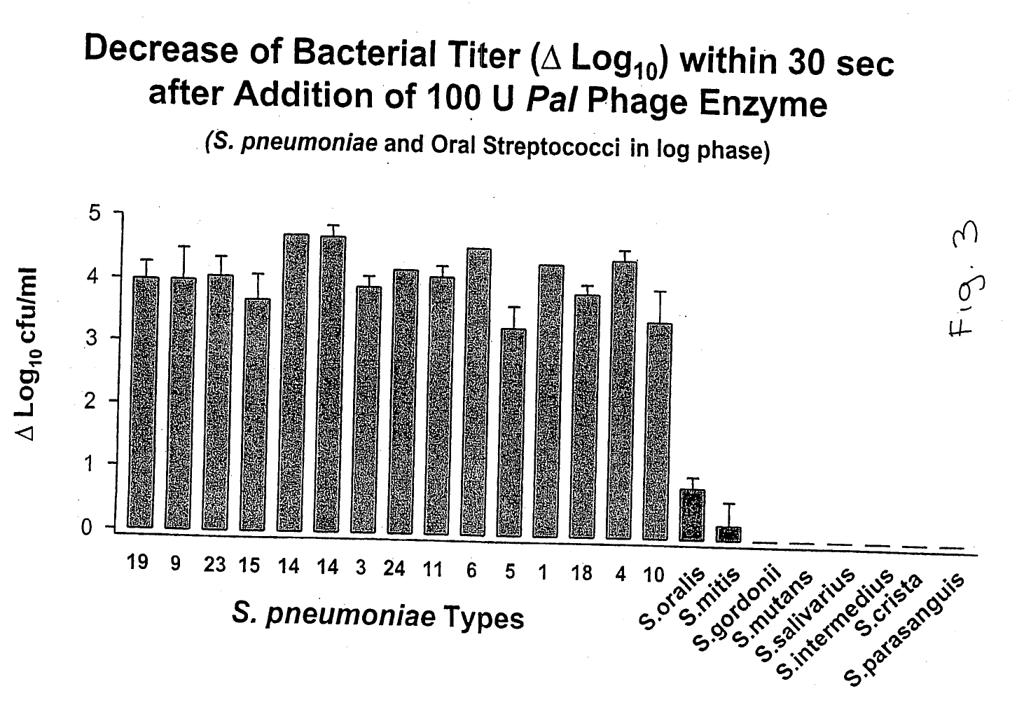

Various serotypes of oral Streptococcl were tested against bacteria-associated lytic enzymes, in particular, the Pal enzyme. A variety of S. pneumoniae type bacteria were also included in the test. Pal enzyme were used at a concentration of 100 U of the purified enzyme. As can be seen in FIG. 3 all S. pneumoniae serotypes are killed (˜4 logs) within the 30 seconds of exposure. Of the oral streptococci tested, only S. oralis and S. mitis show low sensitivity to the Pal enzyme.

### EXAMPLE 9

Susceptibility of Stationary Phase Bacteria to Lytic Enzyme

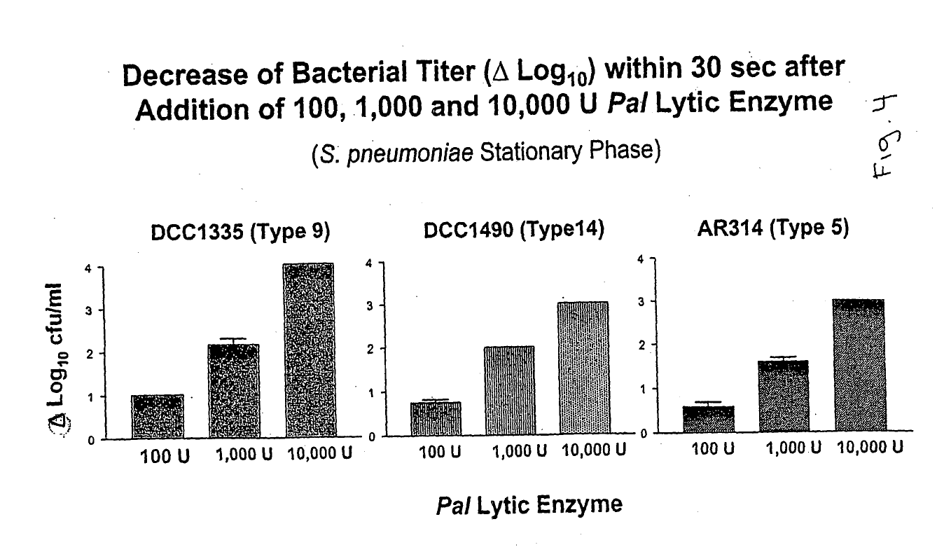

In order to confirm that activities of lytic enzymes are independent of bacterial growth, several serotypes of S. pneumoniae at stationary phase of growth were tested against lytic enzymes. In particular, 3 strains of Pal lytic enzyme were used against 3 serotypes of S. pneumoniae. The results show that all bacterial strains tested against Pal enzyme were killed in 30 seconds (see FIG. 4). An approximately 2-log drop in viability of the bacteria occurred with 1,000 U of enzyme, as opposed to about 34 log drop in the viability with 10,000 units.

### EXAMPLE 10

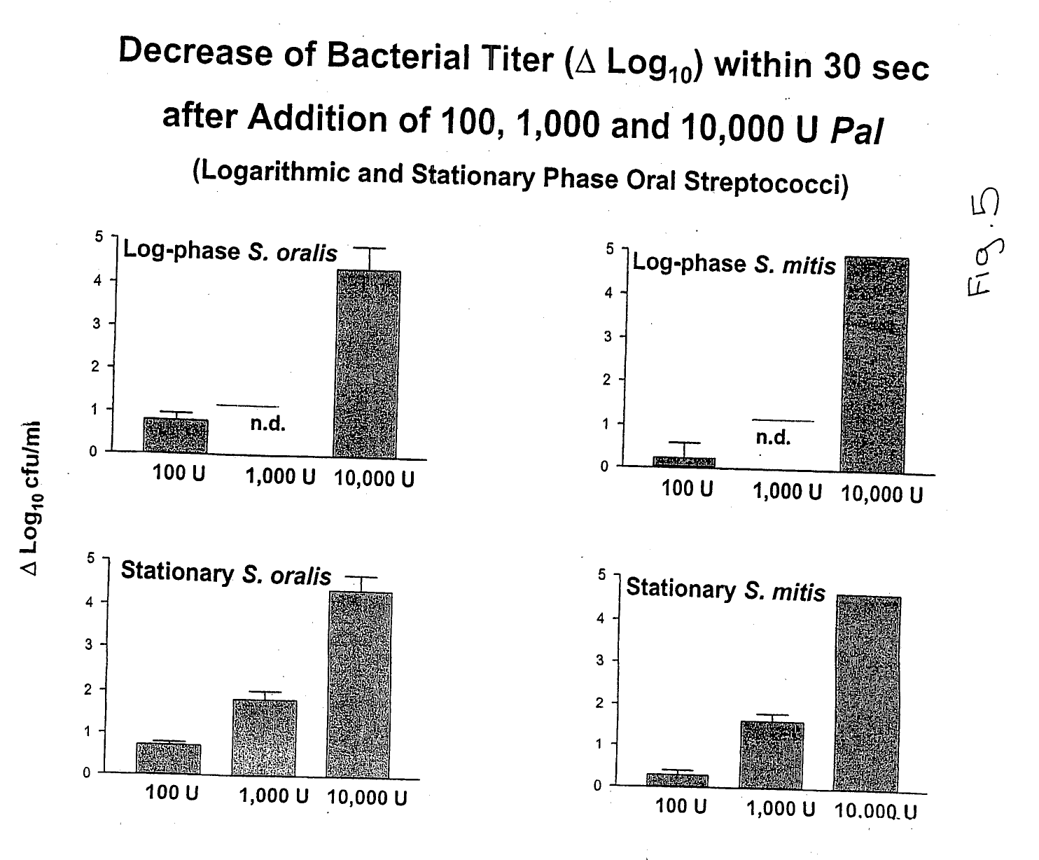

Effect of Pal Lytic Enzyme on Log-Phase and Stationary Phase Oral Streptococci. Streptococci oralis and Streptococcimitis in log or stationary phases of growth were treated with different concentrations of the Pal lytic enzyme. Viability was measured after 30 seconds. Results, as shown in FIG. 5, indicate that both bacterial species were equally sensitive to the Pal enzyme in both log or stationary phases of growth.

In all of the uses for the enzyme, the form of the enzyme may be “natural,” formed by recombinant or “genetically engineered” means, and may be a shuffled, chimeric or otherwise altered enzyme. A holin protein may be used in any of the illnesses discussed, and more than one enzyme may be used in each composition.

Each publication cited herein is incorporated by reference in its entirety. Any part of Harrison's Principles of Internal Medicine, 15^{th} Edition and Robins Pathologic Basis of Infectious Diseases from which much of the background information was obtained which was not properly cited is purely an oversight.

Many modifications and variations of the present disclosure are possible in light of the above teachings. It is, therefore, to be understood within the scope of the appended claims the disclosure may be protected otherwise than as specifically described.

What we claim is:

1) A method of treating respiratory bacterial infection, comprising
administering an effective amount of a therapeutic agent, said therapeutic agent comprising:
a) an effective amount of at least one lytic enzyme genetically coded for by at least one bacteriophage specific for a specific bacteria causing said respiratory bacterial infection, said at least one specific said lytic enzyme having the ability to specifically digest a cell wall of said specific bacteria; and
b) a pharmaceutically acceptable carrier for delivering said at least one lytic enzyme to the mouth, nose, throat or lungs.

2) A composition for treating exposure to a respiratory bacteria, comprising:
a) an effective amount of at least one lytic enzyme genetically coded for by at least one bacteriophage specific for a specific bacteria causing said respiratory bacterial infection, said at least one specific said lytic enzyme having the ability to specifically digest a cell wall of said specific bacteria, and
b) a pharmaceutically acceptable carrier for delivering said at least one lytic enzyme to the mouth, nose, throat or lungs.

3) A composition for treating a Streptococcal Group A respiratory infection, comprising:
a) an effective amount of at least one lytic enzyme genetically coded for by at least one bacteriophage specific for Streptococcus Group A causing said respiratory bacterial infection, said at least one specific said lytic enzyme having the ability to specifically digest a cell wall of said Streptococcus Group A; and
b) a pharmaceutically acceptable carrier for delivering said at least one lytic enzyme to the mouth, nose, throat or lungs,
wherein said pharmaceutically acceptable carrier is selected from the group consisting of a lozenge, troche, candy, chewing gum, tablet, powder, and aerosol.

4) A composition for treating a Streptococcal Group A respiratory infection, comprising:
a) an effective amount of at least one lytic enzyme genetically coded for by at least one bacteriophage specific for Streptococcus Group A causing said respiratory bacterial infection, said at least one specific said lytic enzyme having the ability to specifically digest a cell wall of said Streptococcus Group A; and
b) a pharmaceutically acceptable carrier for delivering said at least one lytic enzyme to the mouth, nose, throat or lungs,
wherein said pharmaceutically acceptable carrier is selected from the group consisting of a liquid, ointment, and spray.

5) A composition for treating a H. influenzae respiratory infection, comprising:
a) an effective amount of at least one lytic enzyme genetically coded for by at least one bacteriophage specific for H. influenzae causing said respiratory bacterial infection, said at least one specific said lytic enzyme having the ability to specifically digest a cell wall of said H. influenzae; and
b) a pharmaceutically acceptable carrier for delivering said at least one lytic enzyme to the mouth, nose, throat or lungs,
wherein said pharmaceutically acceptable carrier is selected from the group consisting of a lozenge, troche, candy, chewing gum, tablet, powder, and aerosol.

6) A composition for treating a H. influenzae respiratory infection, comprising:
a) an effective amount of at least one lytic enzyme genetically coded for by at least one bacteriophage specific for H. influenzae causing said respiratory bacterial infection, said at least one specific said lytic enzyme having the ability to specifically digest a cell wall of said H. influenzae; and
b) a pharmaceutically acceptable carrier for delivering said at least one lytic enzyme to the mouth, nose, throat or lungs,
wherein said pharmaceutically acceptable carrier is selected from the group consisting of a liquid, ointment, and spray.

7) A composition for treating a Streptococcus pneumoniae respiratory infection, comprising:
a) an effective amount of at least one lytic enzyme genetically coded for by at least one bacteriophage specific for Streptococcus pneumoniae causing said respiratory bacterial infection, said at least one specific said lytic enzyme having the ability to specifically digest a cell wall of said Streptococcus pneumoniae; and
b) a pharmaceutically acceptable carrier for delivering said at least one lytic enzyme to the mouth, nose, throat or lungs,
wherein said pharmaceutically acceptable carrier is selected from the group consisting of a lozenge, troche, candy, chewing gum, tablet, powder, and aerosol.

8) A composition for treating a Streptococcus pneumoniae respiratory infection, comprising:
a) an effective amount of at least one lytic enzyme genetically coded for by at least one bacteriophage specific for Streptococcus pneumoniae causing said respiratory bacterial infection, said at least one specific said lytic enzyme having tie ability to specifically digest a cell wall of said Streptococcus pneumoniae; and
b) a pharmaceutically acceptable carrier for delivering said at least one lytic enzyme to the mouth, nose, throat or lungs,
wherein said pharmaceutically acceptable carrier is selected from the group consisting of a liquid, ointment, and spray.

9) A composition for treating a Bacillus anthracis respiratory infection, comprising:
a) an effective amount of at least one lytic enzyme genetically coded for by at least one bacteriophage specific for Bacillus anthracis causing said respiratory bacterial infection, said at least one specific said lytic enzyme having the ability to'specifically digest a cell wall of said Bacillus anthracis; and
b) a pharmaceutically acceptable carrier for delivering said at least one lytic enzyme to the mouth, nose, throat or lungs.
wherein said pharmaceutically acceptable carrier is selected from the group consisting of a lozenge, troche, candy, chewing gum, tablet, powder, and aerosol.

10) A composition for treating a Bacillus anthracis respiratory infection, comprising:
a) an effective amount of at least one lytic enzyme genetically coded for by at least one bacteriophage specific for Bacillus anthracis causing said respiratory bacterial infection, said at least one specific said lytic enzyme having the ability to specifically digest a cell wall of said Bacillus anthracis; and
b) a pharmaceutically acceptable carrier for delivering said at least one lytic enzyme to the mouth, nose, throat or lungs.
wherein said pharmaceutically acceptable carrier is selected from the group consisting of a liquid, ointment, and spray.

11) A composition for treating a respiratory infection, comprising:
a) an effective amount of at least one lytic enzyme genetically coded for by at least one bacteriophage specific for a specific bacteria selected from the group consisting of Mycobacterium bovis, Mycobacterium tuberculosis, Corynebacterium diphtheria, Bordetella pertussis, Streptococcus pyogenes, Staphylococcus aureus, Staphylococcus epidermidis, Klebsiella pneumoniae, Klebsiella rhinoscleromatis, Proteus mirabilis, L. pneumophilia, A. baumannii, A. calcoaceticus, Acinetobacter genospecies 3, Acinetobacter 13Tu, Burkholderia cepacia, Strenotrophomonas maltophilia, Burkholderia pseudomallei, Coxiella burnetti, P. melaningenica, Fusobacterium spp., Prevotella spp., F. nucleatum, Peptostreptococcus spp., Klebsiella ozaenae, Eikenella corrodens, Actinobacillus actinomycetemcomitans, and Cardiobacterium hominis causing said respiratory bacterial infection, wherein said at least one said specific said lytic enzyme is specific for and has the ability to digest a cell wall of one of said bacteria and is coded for by the same said bacteria having the capability of infecting said bacteria being divested and
b) a pharmaceutically acceptable carrier for delivering said at least one lytic enzyme to the mouth, nose, throat or lungs,
wherein said pharmaceutically acceptable carrier is selected from the group consisting of a lozenge, troche, candy, chewing gum, tablet, powder, and aerosol.

12) A composition for treating a respiratory infection, comprising:
a) an effective amount of at least one lytic enzyme genetically coded for by at least one bacteriophage specific for a specific bacteria selected from the group consisting of Mycobacterium bovis, Corynebacterium diphtheria, Bordetella pertussis, Streptococcus pyogenes, Staphylococcus epidermidis, Klebsiella pneumoniae, Klebsiella rhinoscleromatis, Proteus mirabilis, L. pneumophilia, A. baumanni, A. calcoaceticus, Acinetobacter genospecies 3, Acinetobacter 13TU, Burkholderia cepacia, Strenotrophomonas maltophilia, Burkholderia pseudomallei Coxiella burnetti, P. melaningenica, Fusobacterium spp., Prevotella spp., F. nucleatum, Peptostreptococcus spp., Klebsiella ozaenae, Eikenella corrodens, Actinobacillus actinomycetemcomitans, and Cardiobacterium hominis causing said respiratory bacterial infection, wherein said at least one said specific said lytic enzyme is specific for and has the ability to digest a cell wall of one of said bacteria and is coded for by the same said bacteria having the capability of infecting said bacteria being digested, and
b) a pharmaceutically acceptable carrier for delivering said at least one lytic enzyme to the mouth, nose, throat or lungs,
wherein said pharmaceutically acceptable carrier is selected from the group consisting of a liquid, ointment, and spray.

13) A method for treating a respiratory infection, comprising:
administering an effective amount of a therapeutic agent, said therapeutic agent comprising:
a) and effective amount of at least one lytic enzyme genetically coded for by at least one bacteriophage specific for a specific bacteria selected from the group consisting of Mycobacterium bovis, Corynebacterium diphtheria, Bordetella pertussis, Streptococcus pyogenes, Staphylococcus epidermidis, Klebsiella pneumoniae, Klebsiella rhinoscleromatis, Proteus mirabilis, L. pneumophilia, A. baumanni, A. calcoaceticus, Acinetobacter genospecies 3, Acinetobacter 13TU, Burkholderia cepacia, Strenotrophomonas maltophilia, Burkholderia pseudomallei, Coxiella burnetti, P. melaningenica, Fusobacterium spp., Prevotella spp., F. nucleatum, Peptostreptococcus spp., Klebsiella ozaenae, Eikenella corrodens, Actinobacillus actinomycetemcomitans, and Cardiobacterium hominis causing said respiratory bacterial infection, wherein said at least one said specific said lytic enzyme is specific for and has the ability to digest a cell wall of one of said bacteria and is coded for by the same said bacteria having the capability of infecting said bacteria being digested, and
b) a pharmaceutically acceptable carrier for delivering said at least one lytic enzyme to the mouth, nose, throat or lungs.

14) A composition for treating a Staphylococcus aureus respiratory infection, comprising:
a) an effective amount of at least one lytic enzyme genetically coded for by at least one bacteriophage specific for said Staphylococcus aureus causing said respiratory bacterial infection, said at least one specific said lytic enzyme having the ability to specifically digest a cell wall of said Staphylococcus aureus; and
b) a pharmaceutically acceptable carrier for delivering said at least one lytic enzyme to the mouth, nose, throat or lungs.

15) A method of treating Actinobacillus lignieressi infections of tongue tissue in cattle, comprising:
administering an effective amount of a therapeutic agent to a mouth, tongue or throat of said cattle, said therapeutic agent comprising:
a) an effective amount of at least one lytic enzyme genetically coded for by at least one bacteriophage specific for Actinobacillus lignieressi, said at least one specific said lytic enzyme having the ability to specifically digest a cell wall of said Actinobacillus lignieressi; and
b) a pharmaceutically acceptable carrier for delivering said at least one lytic enzyme to the mouth, nose, or throat.

16) A composition for treating exposure to Actinobacillus lignieressi, comprising:
a) an effective amount of at least one lytic enzyme genetically coded for by at least one bacteriophage specific for said Actinobacillus lignieressi causing said respiratory bacterial infection, said at least one specific said lytic enzyme having the ability to specifically digest a cell wall of said Actinobacillus lignieressi; and
b) a pharmaceutically acceptable carrier for delivering said at least one lytic enzyme to the mouth, nose, or throat or throat.

17) A method of treating Streptococcus equi, subsp. equi infections causing strangles in a horse, comprising:
administering an effective amount of a therapeutic agent to a mouth, tongue or throat of said horse, said therapeutic agent comprising:
a) an effective amount of at least one lytic enzyme genetically coded for by at least one bacteriophage specific for Streptococcus equi subsp. equi, said at least one specific said lytic enzyme having the ability to specifically digest a cell wall of said Streptococcus equi, subsp. equi; and
b) a pharmaceutically acceptable carrier for delivering said at least one lytic enzyme to the mouth, nose, or throat.

18) A composition for treating exposure to Streptococcus equi, subsp. equi, comprising:
a) an effective amount of at least one lytic enzyme genetically coded for by at least one bacteriophage specific for said Streptococcus equi subsp.equi causing said respiratory bacterial infection, said at least one specific said lytic enzyme having the ability to specifically digest a cell wall of said Streptococcus equi, subsp. equi; and
b) a pharmaceutically acceptable carrier for delivering said at least one lytic enzyme to the mouth, nose, or throat or throat.

19) A method of treating a bacterial infection causing atrophic rhinitis in pigs, comprising:
administering an effective amount of a therapeutic agent to a mouth, tongue or throat of said pig, said therapeutic agent comprising:
a) an effective amount of at least one lytic enzyme genetically coded for by a specific bacteriophage specific for a bacteria infecting said pig, wherein the bacteria to be treated is selected from the group consisting of Bordetella bronchiseptica, Pasteurella multocida, Haemophilus parasuis, and combinations thereof, wherein said at least one said lytic enzyme is specific for and has the ability to digest a cell wall of one of said bacteria and is coded for by the same said bacteriophage capable of infecting said bacteria being digested; and b) a pharmaceutically acceptable carrier for delivering said at least one lytic enzyme to the mouth, nose, or throat.

20) A composition for treating exposure to a bacteria causing atrophic rhinitis in pigs, comprising:
a) an effective amount of at least one lytic enzyme genetically coded for by a specific bacteriophage specific for a bacteria infecting said pig, wherein the bacteria to be treated is selected from the group consisting of Bordetella bronchiseptica, Pasteurella multocida, Haemophilus parasuis, and combinations thereof, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to the mouth, nose, or throat.

21) A method of treating a bacterial infection causing bacterial rhinitis in dogs, comprising:
administering an effective amount of a therapeutic agent to a mouth, tongue or throat of said dog, said therapeutic agent comprising:
a) an effective amount of at least one lytic enzyme genetically coded for by a specific bacteriophage specific for a bacteria infecting said dog, wherein the bacteria to be treated is selected from the group consisting of Bordetella bronchiseptica, E. coli and Pasteurella multocida, and combinations thereof, wherein said at least one said lytic enzyme is specific for and has the ability to digest a cell wall of one of said bacteria and is coded for by the same said bacteriophage capable of infecting said bacteria being digested; and b) a pharmaceutically acceptable carrier for delivering said at least one lytic enzyme to the mouth, nose, or throat.

22) A composition for treating exposure to a bacteria causing bacterial rhinitis in dogs, comprising:
a) an effective amount of at least one lytic enzyme genetically coded for by a specific bacteriophage specific for a bacteria infecting said dog, wherein the bacteria to be treated is selected from the group consisting of Bordetella bronchiseptica, E. coli, Pasteurella multocida, and combinations thereof, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to the mouth, nose, or throat.

23) A method of treating a bacterial infection causing bacterial supperative rhinitis in felines, comprising:
administering an effective amount of a therapeutic agent to a mouth, tongue or throat of said feline, said therapeutic agent comprising:
a) an effective amount of at least one lytic enzyme genetically coded for by a specific bacteriophage specific for a bacteria infecting said feline, wherein the bacteria to be treated is selected from the group consisting of Pasteurella multocida, Bordetella bronchiseptica, Streptococcus sp., Mycoplasma felis, and combinations thereof, wherein said at least one said lytic enzyme is specific for and has the ability to digest a cell wall of one of said bacteria and is coded for by the same said bacteriophage capable of infecting said bacteria being digested;
and b) a pharmaceutically acceptable carrier for delivering said at least one lytic enzyme to the mouth, nose, or throat.

24) A composition for treating exposure to a bacteria causing bacterial supperative rhinitis in felines, comprising:
a) an effective amount of at least one lytic enzyme genetically coded for by a specific bacteriophage specific for a bacteria infecting said feline, wherein the bacteria to be treated is selected from the group consisting of Pasteurella multocida, Bordetella bronchiseptica, Streptococcus sp., Mycoplasma felis, and combinations thereof, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to the mouth, nose, or throat.

25) A method of treating Fusobacterium necrophorum in feedlot cattle, comprising:
administering an effective amount of a therapeutic agent to a mouth, tongue or throat of said feedlot cattle, said therapeutic agent comprising:
a) an effective amount of at least one lytic enzyme genetically coded for by a specific bacteriophage specific for Fusobacterium necrophorum, wherein said at least one said lytic enzyme has the ability to digest a cell wall of said Fuscobacterium necrophorum; and b) a pharmaceutically acceptable carrier for delivering said at least one lytic enzyme to the mouth, nose, or throat.

26) A composition for treating feedlot cattle exposed to Fusobacterium necrophorum, comprising:
a) an effective amount of at least one lytic enzyme genetically coded for by a specific bacteriophage specific for Fusobacterium necrophorum, wherein said at least one said lytic enzyme has the ability to digest a cell wall of said Fuscobacterium necrophorum; and mouth, nose, or throat.

27) A method of treating canines suffering from infectious tracheobronchitis caused by Bordetella bronchiseptica, comprising:
administering an effective amount of a therapeutic agent to a mouth, tongue or throat of canines, said therapeutic agent comprising:
a) an effective amount of at least one lytic enzyme genetically coded for by at least one bacteriophage specific for Bordetella bronchiseptica causing said bacterial infection, said at least one specific said lytic enzyme having the ability to specifically digest a cell wall of said Bordetella bronchiseptica; and
b) a pharmaceutically acceptable carrier for delivering said at least one lytic enzyme to the mouth, nose, or throat.

28) A composition for treating canines suffering from infectious tracheobronchitis caused by Bordetella bronchiseptica, comprising:
a) an effective amount of at least one lytic enzyme genetically coded for by at least one bacteriophage specific for Bordetella bronchiseptica causing said bacterial infection, said at least one specific said lytic enzyme having the ability to specifically digest a cell wall of said Bordetella bronchiseptica; and
b) a pharmaceutically acceptable carrier for delivering said at least one lytic enzyme to the mouth, nose, or throat.

29) A method of treating supperative bronchopneumonia in domestic animals, comprising:
administering an effective amount of a therapeutic agent, said therapeutic agent comprising:
a) an effective amount of at least one lytic enzyme genetically coded for by a specific bacteriophage specific for a bacteria infecting said animals, wherein the bacteria to be treated is selected from the group consisting of Pasteurella multocida, Bordetella bronchiseptica, Arcanobacterium pyogenes, Streptococcus spp., E. coli, and combinations thereof, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to the mouth, nose, or throat.

30) A method of treating supperative bronchopneumonia in domestic animals, comprising:
administering an effective amount of a therapeutic agent, said therapeutic agent comprising:
a) an effective amount of at least one lytic enzyme genetically coded for by a specific bacteriophage specific for Arcanobacterium pyogenes infecting said animals, wherein said at least one said lytic enzyme is specific for and has the ability to digest a cell wall of said Arcanobacterium pyogenes; and
b) a pharmaceutically acceptable carrier for delivering said at least one lytic enzyme to the mouth, nose, or throat.

31) A method of treating a bacterial infection causing fibrinous bronchopneumonia in domestic animals, comprising:
administering an effective amount of a therapeutic agent to a mouth, tongue or throat of a domestic animal, said therapeutic agent comprising:
a) an effective amount of at least one lytic enzyme genetically coded for by a specific bacteriophage specific for a bacteria infecting said domestic animal, wherein the bacteria to be treated is selected from the group consisting of Pasteurella haemolytica, Haemophilus somnus, Actinobacillus pleuropneumoniae, Mycoplasma mycoides, and combinations thereof, wherein said at least one said lytic enzyme is specific for and has the ability to digest a cell wall of one of said bacteria and is coded for by the same said bacteriophage capable of infecting said bacteria being digested; and b) a pharmaceutically acceptable carrier for delivering said at least one lytic enzyme to the mouth, nose, or throat.

32) A composition for treating exposure to a bacteria causing fibrinous bronchopneumonia in domestic animals, comprising:
a) an effective amount of at least one lytic enzyme genetically coded for by a specific bacteriophage specific for a bacteria infecting said domestic animals, wherein the bacteria to be treated is selected from the group consisting of Pasteurella haemolytica, Haemophilus somnus, Actinobacillus pleuropneumoniae, Mycoplasma mycoides, and combinations thereof, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to the mouth, nose, or throat.

33) A method of treating pigs for pleuropneumoniae caused by Actinobacillus pleuropneumoniae, said method comprising:
administering an effective amount of a therapeutic agent to a mouth, tongue, throat or lung of said pigs, said therapeutic agent comprising:
a) an effective amount of at least one lytic enzyme genetically coded for by at least one bacteriophage specific for Actinobacillus pleuropneumoniae, said at least one specific said lytic enzyme having the ability to specifically digest a cell wall of said Actinobacillus pleuropneumoniae; and
b) a pharmaceutically acceptable carrier for delivering said at least one lytic enzyme to the mouth, nose, throat, or lung.

34) A composition for treating exposure to Actinobacillus pleuropneumoniae, comprising:
a) an effective amount of at least one lytic enzyme genetically coded for by at least one bacteriophage specific for said Actinobacillus pleuropneumoniae causing said respiratory bacterial infection, said at least one specific said lytic enzyme having the ability to specifically digest a cell wall of said Actinobacillus pleuropneumoniae; and
b) a pharmaceutically acceptable carrier for delivering said at least one lytic enzyme to the mouth, nose, or throat or throat.

35) A method of treating granulomatous pneumonia caused by Mycobacterium bovis, said method comprising:
administering an effective amount of a therapeutic agent to a mouth, tongue, throat or lung of animals, said therapeutic agent comprising:
a) an effective amount of at least one lytic enzyme genetically coded for by at least one bacteriophage specific for Mycobacterium bovis, said at least one specific said lytic enzyme to having the ability to specifically digest a cell wall of said Mycobacterium bovis; and
b) a pharmaceutically acceptable carrier for delivering said at least one lytic enzyme to the mouth, nose, throat, or lung.

36) A composition for treating exposure to Mycobacterium bovis, comprising:
a) an effective amount of at least one lytic enzyme genetically coded for by at least one bacteriophage specific for said Mycobacterium bovis, said at least one specific said lytic enzyme having the ability to specifically digest a cell wall of said Mycobacterium bovis; and
b) a pharmaceutically acceptable carrier for delivering said at least one lytic enzyme to the mouth, nose, or throat or throat.

37) A method of treating granulomatous pneumonia in goats caused by Rhodococcus equi, said method comprising:
administering an effective amount of a therapeutic agent to a mouth, tongue, throat or lung of said goats, said therapeutic agent comprising:
a) an effective amount of at least one lytic enzyme genetically coded for by at least one bacteriophage specific for Rhodococcus equi, said at least one specific said lytic enzyme having the ability to specifically digest a cell wall of said Rhodococcus equi; and
b) a pharmaceutically acceptable carrier for-delivering said at least one lytic enzyme to the mouth, nose, throat, or lung.

38) A composition for treating exposure to Rhodococcus equi, comprising:
a) an effective amount of at least one lytic enzyme genetically coded for by at least one bacteriophage specific for said Rhodococcus equi, said at least one specific said lytic enzyme having the ability to specifically digest a cell wall of said Rhodococcus equi; and
b) a pharmaceutically acceptable carrier for delivering said at least one lytic enzyme to the mouth, nose, or throat or lungs.

39) A method of treating a bacterial infection causing pleuropneumoniae in goats, comprising:
administering an effective amount of a therapeutic agent to a mouth, tongue, lungs, or throat of said goats, said therapeutic agent comprising:
a) an effective amount of at least one lytic enzyme genetically coded for by a specific bacteriophage specific for a bacteria infecting said goats, wherein the bacteria to be treated is selected from the group consisting of M. mycoides, spp. capri Mycoplasma strain F38, and combinations thereof, wherein said at least one said lytic enzyme is specific for and has the ability to digest a cell wall of one of said bacteria and is coded for by the same said bacteriophage capable of infecting said bacteria being digested; and
b) a pharmaceutically acceptable-carrier for delivering said at least one lytic enzyme to the mouth, nose, throat, or lungs.

40) A composition for treating goats for exposure to bacteria causing pleuropneumoniae, comprising:
a) an effective amount of at least one lytic enzyme genetically coded for by a specific bacteriophage specific for a bacteria infecting said goats, wherein the bacteria to be treated is selected from the group consisting of, M. mycoides, spp. capri, Mycoplasma strain F38, and combinations thereof, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to the mouth, nose, throat, or lung.

41) A method of treating a bacterial infection in sheep and goats, comprising:
administering an effective amount of a therapeutic agent to a mouth, tongue, throat or lungs of said sheep and goats, said therapeutic agent comprising:
a) an effective amount of at least one lytic enzyme genetically coded for by a specific bacteriophage specific for a bacteria infecting said goats and sheep, wherein the bacteria to be treated is selected from the group consisting of M. ovipneumoniae, M. capriolum, and combinations thereof, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to the mouth, nose, throat, or lungs.

42) A composition for treating goats and sheep for exposure to bacteria causing pneumonia like disease, comprising:
a) an effective amount of at least one lytic enzyme genetically coded for by a specific bacteriophage specific for a bacteria infecting said goats and sheep, wherein the bacteria to be treated is selected from the group consisting of M. ovipneumoniae, M. caprioluyn and combinations thereof, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to the mouth, nose, throat, or lungs.

43) A method of treating porcine pneumoniae caused by Mycoplasma hypopneumoniae, said method comprising
administering an effective amount of a therapeutic agent to a mouth tongue throat or lung of pigs, said therapeutic agent comprising:
a) an effective amount of at least one lytic enzyme genetically coded for by at least one bacteriophage specific for Mycoplasma hypopneumoniae, said at least one specific said lytic enzyme having the ability to specifically digest a cell wall of said Mycoplasma hypopneumoniae; and
b) a pharmaceutically acceptable carrier for delivering said at least one lytic enzyme to the mouth, nose, throat, or lung.

44) A composition for treating exposure to Mycoplasma hypopneumoniae, comprising:
a) an effective amount of at least one lytic enzyme genetically coded for by at least one bacteriophage specific for said Mycoplasma hypopneumoniae, said at least one specific said lytic enzyme having the ability to specifically digest a cell wall of said Mycoplasma hypopneumoniae; and
b) a pharmaceutically acceptable carrier for delivering said at least one lytic enzyme to the mouth, nose, or throat or lungs.

45) A method of treating porcine pneumoniae caused by Streptococcus suis type II, said method comprising
administering an effective amount of a therapeutic agent to a mouth, tongue, throat or lung of pigs, said therapeutic agent comprising:
a) an effective amount of at least one lytic enzyme genetically coded for by at least one bacteriophage specific for Streptococcus suis type II, said at least one specific said lytic enzyme having the ability to specifically digest a cell wall of said Streptococcus suis type II; and
b) a pharmaceutically acceptable carrier for delivering said at least one lytic enzyme to the mouth, nose, throat, or lung.

46) A composition for treating exposure to Streptococcus suis tape II, comprising:
a) an effective amount of at least one lytic enzyme genetically coded for by at least one bacteriophage specific for said Streptococcus suis type II, said at least one specific said lytic enzyme having the ability to specifically digest a cell wall of said Streptococcus suis type II; and
b) a pharmaceutically acceptable carrier for delivering said at least one lytic enzyme to the mouth, nose, or throat or lungs.

47) A method of treating a bacterial infection causing tuberculosis in cats, comprising:
administering an effective amount of a therapeutic agent to a mouth, tongue, throat or lungs of said cats, said therapeutic agent comprising:
a) an effective amount of at least one lytic enzyme genetically coded for by a specific bacteriophage specific for a bacteria infecting said cats, wherein the bacteria to be treated is selected from the group consisting of M. bovis, M. tuberculosis, M. avium, and combinations thereof, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to the mouth, nose, throat, or lungs.

48) A composition for treating cats for exposure to bacteria causing pneumonia, comprising:
a) an effective amount of at least one lytic enzyme genetically coded for by a specific bacteriophage specific for a bacteria infecting cats, wherein the bacteria to be treated is selected from the group consisting of M. bovis, M. tuberculosis, M. avium, and combinations thereof, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to the mouth, nose, throat, or lungs.

49) A method of treating respiratory problems in fowl caused by Mycoplasma gallisepticum, said method comprising
administering an effective amount of a therapeutic agent to a mouth, tongue, throat or lung of said fowl, said therapeutic agent comprising:
a) an effective amount of at least one lytic enzyme genetically coded for by at least one bacteriophage specific for Mycoplasma gallisepticum, said at least one specific said lytic enzyme having the ability to specifically digest a cell wall of said Mycoplasma gallisepticum; and
b) a pharmaceutically acceptable carrier for delivering said at least one lytic enzyme to the mouth, nose, throat, or lung.

50) A composition for treating exposure to Mycoplasma gallisepticum, comprising:
a) an effective amount of at least one lytic enzyme genetically coded for by at least one bacteriophage specific for said Mycoplasma gallisepticum, said at least one specific said lytic enzyme having the ability to specifically digest a cell wall of said Mycoplasma gallisepticum; and
b) a pharmaceutically acceptable carrier for delivering said at least one lytic enzyme to the mouth, nose, or throat or lungs.

51) A method of treating airsacculitis in turkeys caused by Mycoplasma meleagridis, said method comprising
administering an effective amount of a therapeutic agent to a mouth, tongue, throat or lung of said turkeys, said therapeutic agent comprising:
a) an effective amount of at least one lytic enzyme genetically coded for by at least one bacteriophage specific for Mycoplasma meleagridis, said at least one specific said lytic enzyme having the ability to specifically digest a cell wall of said Mycoplasma meleagridis; and
b) a pharmaceutically acceptable carrier for delivering said at least one lytic enzyme to the mouth, nose, throat, or lung.

52) A composition for treating exposure to Mycoplasma meleagridis, comprising:
a) an effective amount of at least one lytic enzyme genetically coded for by at least one bacteriophage specific for said Mycoplasma meleagridis, said at least one specific said lytic enzyme having the ability to specifically digest a cell wall of said Mycoplasma meleagridis; and
b) a pharmaceutically acceptable carrier for delivering said at least one lytic enzyme to the mouth, nose, or throat or lungs.

53) A method of treating infectious synovitis in turkeys and chickens caused by Mycoplasmasynoviae, said method comprising:
administering an effective amount of a therapeutic agent to a mouth, tongue, throat or lung of said turkeys and chickens, said therapeutic agent comprising:
a) an effective amount of at least one lytic enzyme genetically coded for by at least one bacteriophage specific for Mycoplasma synoviae, said at least one specific said lytic enzyme having the ability to specifically digest a cell wall of said Mycoplasma synoviae; and
b) a pharmaceutically acceptable carrier for delivering said at least one lytic enzyme to the mouth, nose, throat, or lung.

54) A composition for treating exposure to Mycoplasma synoviae, comprising:
a) an effective amount of at least one lytic enzyme genetically coded for by at least one bacteriophage specifics for said Mycoplasma synoviae, said at least one specific said lytic enzyme having the ability to specifically digest a cell wall of said Mycoplasma synoviae; and
b) a pharmaceutically acceptable carrier for delivering said at least one lytic enzyme to the mouth, nose, or throat or lungs.

55) A method of treating a respiratory disease in chickens caused by Hemophilus gallinarum, said method comprising
administering an effective amount of a therapeutic agent to a mouth, tongue, throat or lung of said chickens, said therapeutic agent comprising:
a) an effective amount of at least one lytic enzyme genetically coded for by at least one bacteriophage specific for Hemophilus gallinarum, said at least one specific said lytic enzyme having the ability to specifically digest a cell wall of said Hemophilus gallinarum; and
b) a pharmaceutically acceptable carrier for delivering said at least one lytic enzyme to the mouth, nose, throat, or lung.

56) A composition for treating exposure to Hemophilus gallinarum, comprising:
a) an effective amount of at least one lytic enzyme genetically coded for by at least one bacteriophage specific for said Hemophilus gallinarum, said at least one specific said lytic enzyme having the ability to specifically digest a cell wall of said Hemophilus gallinarum; and
b) a pharmaceutically acceptable carrier for delivering said at least one lytic enzyme to the mouth, nose, or throat or lungs.

57) A method of treating pneumoniae in horses caused by a bacterial infection, said method comprising:
administering an effective amount of a therapeutic agent to a mouth, tongue, throat or lungs of said horses, said therapeutic agent comprising:
a) an effective amount of at least one lytic enzyme genetically coded for by a specific bacteriophage specific for a bacteria infecting said horses, wherein the bacteria to be treated is selected from the group consisting of Streptococcus spp., E. coli, Klebsiella pneumoniae, Rhodococcus equi, and combinations thereof, wherein said at least one said lytic enzyme is specific for and has the ability to digest a cell wall of one of said bacteria and is coded for by the same said bacteriophage capable of infecting said bacteria being digested; and b) a pharmaceutically acceptable carrier for delivering said at least one lytic enzyme to the mouth, nose, throat, or lungs.

58) A composition for treating horses for exposure to bacteria causing pneumonia, comprising:
a) an effective amount of at least one lytic enzyme genetically coded for by a specific bacteriophage specific for a bacteria infecting horses, wherein the bacteria to be treated is selected from the group consisting of Streptococcus spp., E. coli, Klebsiella pneumoniae, Rhodococcus equi, and combinations thereof, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to the mouth, nose, throat, or lungs.

59) A method of treating pneumonic asteurellosis in cattle caused by a bacterial infection, said method comprising:
administering an effective amount of a therapeutic agent to a mouth, tongue, throat or lungs of said cattle, said therapeutic agent comprising:
a) an effective amount of at least one lytic enzyme genetically coded for by a specific bacteriophage specific for a bacteria infecting said cattle, wherein the bacteria to be treated is selected from the group consisting of P. haemolytica, P. multocida, and combinations thereof, wherein said at least one said lytic enzyme is specific for and has the ability to digest a cell wall of one of said bacteria and is coded for by the same said bacteriophage capable of infecting said bacteria-being digested; and b) a pharmaceutically acceptable carrier for delivering said at least one lytic enzyme to the mouth, nose, throat, or lungs.

60) A composition for treating cattle for exposure to bacteria causing pneumonic Pasteurellosis, comprising:
a) an effective amount of at least one lytic enzyme genetically coded for by a specific bacteriophage specific for a bacteria infecting cattle, wherein the bacteria to be treated is selected from the group consisting of P. haemolytica, P. multocida, and combinations thereof, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to the mouth, nose, throat, or lungs.

61) Animal feed to treat or prevent bacterial disease, said animal feed comprising:
a) an effective amount of at least one lytic enzyme genetically coded for by a specific bacteriophage specific for a bacteria infecting an animal, wherein said at least one said lytic enzyme is specific for and has the ability to digest a cell wall of said bacteria and is coded for by the same said bacteriophage capable of infecting said bacteria being digested; and
b) a pharmaceutically acceptable animal feed carrier for delivering said at least one lytic enzyme.

62) Animal feed to treat or prevent bacterial disease, wherein said animal feed is produced by the steps of:
a) obtaining an effective amount of at least one lytic enzyme genetically coded for by a specific bacteriophage specific for a bacteria infecting an animal, wherein said at least one said lytic enzyme is specific for and has the ability to digest a cell wall of said bacteria and is coded for by the same said bacteriophage capable of infecting said bacteria being digested;
b) adding said at least one lytic enzyme to a pharmaceutically acceptable carrier for delivering said at least one lytic enzyme; and
c) incorporating said pharmaceutically acceptable carrier containing said at least one lytic enzyme to said animal feed.

63) A method of treating or preventing bacterial infections of animals, said method comprising:
A) adding an effective amount of a therapeutic agent to animal feed, said therapeutic agent comprising:
i) an effective amount of at least one lytic enzyme genetically coded for by a specific bacteriophage specific for a bacteria infecting an animal, wherein said at least one said lytic enzyme is specific for and has the ability to digest a cell wall of said bacteria and is coded for by the same said bacteriophage capable of infecting said bacteria being digested; and
ii) an animal feed carrier for delivering said enzyme to the animal;
B) feeding said animal feed to said animals.

64) Vegetable matter to treat or prevent a bacterial disease, said vegetable matter comprising:
a) an effective amount of at least one lytic enzyme genetically coded for by a specific bacteriophage specific for a bacteria infecting an animal, wherein said at least one said lytic enzyme is specific for and has the ability to digest a cell wall of said bacteria and is coded for by the same said bacteriophage capable of infecting said bacteria being digested; and
b) a pharmaceutically acceptable vegetable matter carrier for delivering said at least one lytic enzyme.

65) Vegetable matter to treat or prevent a bacterial disease, wherein said vegetable matter is produced by the steps of:
a) obtaining an effective amount of at least one lytic enzyme genetically coded for by a specific bacteriophage specific for a bacteria infecting an animal, wherein said at least one said lytic enzyme is specific for and has the ability to digest a cell wall of said bacteria and is coded for by the same said bacteriophage capable of infecting said bacteria being digested;
b) adding said at least one lytic enzyme to a pharmaceutically acceptable carrier for delivering said at least one lytic enzyme; and
c) incorporating said pharmaceutically acceptable carrier containing said at least one lytic enzyme to said vegetable matter.

66) A method of treating or preventing bacterial infections of animals, said method comprising:
adding an effective amount of a therapeutic agent to animal feed, said therapeutic agent comprising:
a) an effective amount of at least one lytic enzyme genetically coded for by a specific bacteriophage specific for a bacteria infecting an animal, wherein said at least one said lytic enzyme is specific for and has the ability to digest a cell wall of said bacteria and is coded for by the same said bacteriophage capable of infecting said bacteria being digested; and
b) a vegetable matter carrier for delivering said enzyme to the animal.

67) A potable liquid to treat or prevent bacterial disease, said animal feed comprising:
a) an effective amount of at least one lytic enzyme genetically coded for by a specific bacteriophage specific for a bacteria infecting an animal, wherein said at least one said lytic enzyme is specific for and has the ability to digest a cell wall of said bacteria and is coded for by the same said bacteriophage capable of infecting said bacteria being digested; and
b) a pharmaceutically acceptable potable liquid carrier for delivering said at least one lytic enzyme.

68) A potable liquid to treat or prevent bacterial disease, wherein said animal feed is produced by the steps of:
a) obtaining an effective amount of at least one lytic enzyme genetically coded for by a specific bacteriophage specific for a bacteria infecting an animal, wherein said at least one said lytic enzyme is specific for and has the ability to digest a cell wall of said bacteria and is coded for by the same said bacteriophage capable of infecting said bacteria being digested;
b) adding said at least one lytic enzyme to a pharmaceutically acceptable carrier for delivering said at least one lytic enzyme; and
c) incorporating said pharmaceutically acceptable carrier containing said at least one lytic enzyme to said potable liquid.

69) A method of treating or preventing bacterial infections of animals, said method comprising:
adding an effective amount of a therapeutic agent to a potable liquid, said therapeutic agent comprising:
a) an effective amount of at least one lytic enzyme genetically coded for by a specific bacteriophage specific for a bacteria infecting an animal, wherein said at least one said lytic enzyme is specific for and has the ability to digest a cell wall of said bacteria and is coded for by the same said bacteriophage capable of infecting said bacteria being digested; and
b) a potable liquid carrier for delivering said enzyme to the animal.

70) A method of treating sialoadenitis comprising:
administering an effective amount of a therapeutic agent to the mouth, tongue and throat, said therapeutic agent comprising:
a) an effective amount of at least one lytic enzyme genetically coded for by at least one bacteriophage specific for a specific bacteria infecting salivary glands, wherein the bacteria to be treated is selected from the group consisting of Staphylococcus aureus, Streptococcus viridans, and combinations thereof, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to said salivary glands.

71) The composition for treating Streptococcus viridans causing a bacterial infection of salivary glands, said composition comprising:
a) an effective amount of at least one lytic enzyme genetically coded for by a specific bacteriophage specific for said Streptococcus viridans infecting said salivary glands wherein said at least one lytic enzyme is specific for and has the ability to digest a cell wall of said Streptococcus viridans, and is coded for by the same said bacteriophage cable of infecting said Streptococcus viridans being digested; and
b) a pharmaceutically acceptable carrier for delivering said at least one lytic enzyme to the salivary glands, mouth, nose or throat.
A composition for treating exposure to a bacteria causing bacterial esophagus, comprising:
a) an effective amount of at least one lytic enzyme genetically coded for by a specific bacteriophage specific for a bacteria infecting an esophagus, wherein the bacteria to be treated is selected from the group consisting of Lactobacillus, B-hemolytic Streptococci, Mycobacterium tuberculosis, and combinations thereof, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to the esophagus.

73) A method of treating exposure to a bacteria causing bacterial esophagus, comprising:
administering an effective amount of a therapeutic agent to salivary glands mouth, or tongue, said therapeutic agent comprising:
a) an effective amount of at least one lytic enzyme genetically coded for by a specific bacteriophage specific for a bacteria infecting an esophagus, wherein the bacteria to be treated is selected from the group consisting of Lactobacillus, B-hemolytic Streptococci, Mycobacterium tuberculosis, and combinations thereof, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to the esophagus.
A composition for treating ulcers caused by H. pylori, said composition comprising:
a) an effective amount of at least one lytic enzyme genetically coded for by a specific bacteriophage specific for said H. pylori infecting a stomach, wherein said at least one said lytic enzyme is specific for and has the ability to digest a cell wall of said H. pylori, said enzyme being coded for by the same said bacteriophage capable of infecting said bacteria being digested; and
b) a pharmaceutically acceptable carrier for delivering said at least one lytic enzyme to the stomach.

74) A method of treating ulcers caused by H. pylori, said method comprising:
administering an effective amount of a therapeutic agent to salivary glands, mouth, or tongue, said therapeutic agent comprising:
a) an effective amount of at least one lytic enzyme genetically coded for by a specific bacteriophage specific for said H. pylori infecting a stomach, wherein said at least one said lytic enzyme is specific for and has the ability to digest a cell wall of one of said H. pylori and is coded for by the same said bacteriophage capable of infecting said H. pylori being digested; and
b) a pharmaceutically acceptable carrier for delivering said at least one lytic enzyme to the stomach.
A composition for treating exposure to a bacteria causing chronic diarrhea, comprising:
a) an effective amount of at least one lytic enzyme genetically coded for by a specific bacteriophage specific for a bacteria infecting an intestinal tract, wherein the bacteria to be treated is selected from the group consisting of G. lambi, Yersinia enterocolitica, C. difficile, Klebsiella pneumoniae, Enterobacter cloacae, and combinations thereof, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to the intestinal tract.

75) A method of treating exposure to a bacteria causing chronic diarrhea, comprising:
administering an effective amount of a therapeutic agent to an intestinal tract, said therapeutic agent comprising:
a) an effective amount of at least one lytic enzyme genetically coded for by a specific bacteriophage specific for a bacteria infecting an intestinal tract, wherein the bacteria to be treated is selected from the group consisting of G. lambi, Yersinia enterocolitica, C. difficile, Klebsiella pneumoniae, Enterobacter cloacae, and combinations thereof, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to the intestinal tract.

76) A method of treating exposure to a bacteria causing secretory diarrhea, comprising:
administering an effective amount of a therapeutic agent to an intestinal tract, said therapeutic agent comprising:
a) an effective amount of at least one lytic enzyme genetically coded for by a specific bacteriophage specific for a bacteria infecting an intestinal tract, wherein the bacteria to be treated is selected from the group consisting of Vibrio cholera, Bacillus cereus, Clostridium perfringens, and combinations thereof, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to the intestinal tract.
A composition for treating exposure to a bacteria causing secretory diarrhea, comprising:
a) an effective amount of at least one lytic enzyme genetically coded for by a specific bacteriophage specific for a bacteria infecting an intestinal tract, wherein the bacteria to be treated is selected from the group consisting of Vibrio cholerae, Bacillus cereus, Clostridia perfringens, and combinations thereof, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to the intestinal tract.
A method of treating exposure to E. coli causing secretory diarrhea, comprising:
administering an effective amount of a therapeutic agent to an intestinal tract, said therapeutic agent comprising:
a) an effective amount of at least one lytic enzyme genetically coded for by a specific bacteriophage specific for E. coli infecting an intestinal tract, wherein said at least one said lytic enzyme is specific for and has the ability to digest a cell wall of one of said E. coli and is coded for by the same said bacteriophage capable of infecting said bacteria being digested; and
b) a pharmaceutically acceptable carrier for delivering said at least one lytic enzyme to the intestinal tract.
A composition for treating exposure to E. coli causing secretory diarrhea, comprising:
a) an effective amount of at least one lytic enzyme genetically coded for by a specific bacteriophage specific for said E. coli infecting an intestinal tract, wherein said at least one said lytic enzyme is specific for and has the ability to digest a cell wall of one of said E. coli and is coded for by the same said bacteriophage capable of infecting said E. coli being digested; and
b) a pharmaceutically acceptable carrier for delivering said at least one lytic enzyme to the intestinal tract.
A method of treating exposure to Campylobacter causing secretory diarrhea, comprising:
administering an effective amount of a therapeutic agent to an intestinal tract, said therapeutic agent comprising:
a) an effective amount of at least one lytic enzyme genetically coded for by a specific bacteriophage specific for Campylobacter infecting an intestinal tract, wherein said at least one said lytic enzyme is specific for and has the ability to digest a cell wall of one of said Campylobacter and is coded for by the same said bacteriophage capable of infecting said bacteria being digested; and
b) a pharmaceutically acceptable carrier for delivering said at least one lytic enzyme to the intestinal tract.
A composition for treating exposure to Campylobacter causing secretory diarrhea, comprising:
a) an effective amount of at least one lytic enzyme genetically coded for by a specific bacteriophage specific for said Campylobacter infecting an intestinal tract, wherein said at least one said lytic enzyme is specific for and has the ability to digest a cell wall of one of said Campylobacter and is coded for by the same said bacteriophage capable of infecting said Campylobacter being digested; and
b) a pharmaceutically acceptable carrier for delivering said at least one lytic enzyme to the intestinal tract.
A method of treating exposure to Salmonella causing secretory diarrhea, comprising:
administering an effective amount of a therapeutic agent to an intestinal tract, said therapeutic agent comprising:
a) an effective amount of at least one lytic enzyme genetically coded for by a specific bacteriophage specific for Salmonella infecting an intestinal tract, wherein said at least one said lytic enzyme is specific for and has the ability to digest a cell wall of one of said Salmonella and is coded for by the same said bacteriophage capable of infecting said bacteria being digested; and
b) a pharmaceutically acceptable carrier for delivering said at least one lytic enzyme to the intestinal tract.
A composition for treating exposure to Salmonella causing secretory diarrhea, comprising:
a) an effective amount of at least one lytic enzyme genetically coded for by a specific bacteriophage specific for said Salmonella infecting an intestinal tract, wherein said at least one said lytic enzyme is specific for and has the ability to digest a cell wall of one of said Salmonella and is coded for by the same said bacteriophage capable of infecting said Salmonella being digested; and
b) a pharmaceutically acceptable carrier for delivering said at least one lytic enzyme to the intestinal tract.
A method of treating exposure to Shigella causing secretory diarrhea, comprising:
administering an effective amount of a therapeutic agent to an intestinal tract, said therapeutic agent comprising:
a) an effective amount of at least one lytic enzyme genetically coded for by a specific bacteriophage specific for Shigella infecting an intestinal tract, wherein said at least one said lytic enzyme is specific for and has the ability to digest a cell wall of one of said Shigella and is coded for by the same said bacteriophage capable of infecting said bacteria being digested; and
b) a pharmaceutically acceptable carrier for delivering said at least one lytic enzyme to the intestinal tract.
A composition for treating exposure to Shigella causing secretory diarrhea, comprising:
a) an effective amount of at least one lytic enzyme genetically coded for by a specific bacteriophage specific for said Shigella infecting an intestinal tract, wherein said at least one said lytic enzyme is specific for and has the ability to digest a cell wall of one of said Shigella and is coded for by the same said bacteriophage capable of infecting said Shigella being digested; and
b) a pharmaceutically acceptable carrier for delivering said at least one lytic enzyme to the intestinal tract.

77) A method of treating exposure to a bacteria causing secretory diarrhea, comprising:
administering an effective amount of a therapeutic agent to an intestinal tract, said therapeutic agent comprising:
a) an effective amount of at least one lytic enzyme genetically coded for by a specific bacteriophage specific for a bacteria infecting an intestinal tract, wherein the bacteria to be treated is selected from the group consisting of Shigella dysenteriae, Shigella flexneri, Shigella boydii, Shigella sonnei, and combinations thereof, wherein said at least one said lytic enzyme is specific for and has the ability to direst a cell wall of one of said bacteria and is coded for by the same said bacteriophage capable of infecting said bacteria being digested; and
b) a pharmaceutically acceptable carrier for delivering said at least one lytic enzyme to the intestinal tract.
A composition for treating exposure to a bacteria causing secretory diarrhea, comprising:
a) an effective amount of at least one lytic enzyme genetically coded for by a specific bacteriophage specific for a bacteria infecting an intestinal tract, wherein the bacteria to be treated is selected from the group consisting of Shigella dysenteriae, Shigella flexneri, Shigella boydii, Shigella sonnei, and combinations thereof, wherein said at least one said lytic enzyme is specific for and has die ability to digest a cell wall of one of said bacteria and is coded for by the same said bacteriophage capable of infecting said bacteria being digested; and
b) a pharmaceutically acceptable carrier for delivering said at least one lytic enzyme to the intestinal tract.

78) A method of treating exposure to a bacteria causing diarrhea, comprising:
administering an effective amount of a therapeutic agent to an intestinal tract, said therapeutic agent comprising:
a) an effective amount of at least one lytic enzyme genetically coded for by a specific bacteriophage specific for a bacteria infecting an intestinal tract, wherein the bacteria to be treated is selected from the group consisting of V. cholerae, Y. pseudotuberculosis, and combinations thereof, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to the intestinal tract.
A composition for treating exposure to a bacteria causing secretory diarrhea, comprising:
a) an effective amount of at least one lytic enzyme genetically coded for by a specific bacteriophage specific for a bacteria infecting an intestinal tract, wherein the bacteria to be treated is selected from the group consisting of V. chlolerae, Y. pseudotubeiculogis, and combinations thereof, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to the intestinal tract.

79) A method of treating exposure to a bacteria causing diarrhea, comprising:
administering an effective amount of a therapeutic agent to an intestinal tract, said therapeutic agent comprising:
a) an effective amount of at least one lytic enzyme genetically coded for by a specific bacteriophage specific for a bacteria infecting an intestinal tract, wherein the bacteria to be treated is selected from the group consisting of Salmonella enteritidis, Salmonella typhimurium, Salmonella paratyphi, and combinations thereof, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to the intestinal tract.
A composition for treating exposure to a bacteria causing secretory diarrhea, comprising:
a) an effective amount of at least one lytic enzyme genetically coded for by a specific bacteriophage specific for a bacteria infecting an intestinal tract, wherein the bacteria to be treated is selected from the group consisting of Salmonella enteritidis, Salmonella typhimurium, Salmonella paratyphi, and combinations thereof, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to the intestinal tract.

80) A method of treating exposure to Campylobacter jejuni causing diarrhea, comprising:
administering an effective amount of a therapeutic agent to an intestinal tract, said therapeutic agent comprising:
a) an effective amount of at least one lytic enzyme genetically coded for by a specific bacteriophage specific for said Campylobacter jejuni infecting an intestinal tract, wherein said at least one said lytic enzyme is specific for and has the ability to digest a cell wall of one of said Campylobacter jejuni and is coded for by the same said bacteriophage capable of infecting said bacteria being digested; and
b) a pharmaceutically acceptable carrier for delivering said at least one lytic enzyme to the intestinal tract.
A composition for treating exposure to Campylobacter jejuni, comprising:
a) an effective amount of at least one lytic enzyme genetically coded for by a specific bacteriophage specific for said Campylobacter jejuni infecting an intestinal tract, wherein said at least one said lytic enzyme is specific for and has the ability to digest a cell wall of one of said Campylobacter jejuni and is coded for by the same said bacteriophage capable of infecting said Campylobacter jejuni being digested; and
b) a pharmaceutically acceptable carrier for delivering said at least one lytic enzyme to the intestinal tract.

81) A method of treating exposure to Bacteroides causing diarrhea, comprising:
administering an effective amount of a therapeutic agent to an intestinal tract, said therapeutic agent comprising:
a) an effective amount of at least one lytic enzyme genetically coded for by a specific bacteriophage specific for said Bacteroides infecting an intestinal tract, wherein said at least one said lytic enzyme is specific for and has the ability to digest a cell wall of one of said Bacteroides and is coded for by the same said bacteriophage capable of infecting said bacteria being digested; and
b) a pharmaceutically acceptable carrier for delivering said at least one lytic enzyme to the intestinal tract.
A composition for treating exposure to Bacteroides, comprising:
a) an effective amount of at least one lytic enzyme genetically coded for by a specific bacteriopage specific for said Bacteroides infecting an intestinal tract, wherein said at least one said lytic enzyme is specific for and has the ability to digest a cell wall of one of said Bacteroides and is coded for by the same said bacteriophage capable of infecting said Bacteroides being digested; and
b) a pharmaceutically acceptable carrier for delivering said at least one lytic enzyme to the intestinal tract.

82) A method of treating exposure to Edwardsiella causing gastroenteritis, comprising:
administering an effective amount of a therapeutic agent to an intestinal tract, said therapeutic agent comprising:
a) an effective amount of at least one lytic enzyme genetically coded for by a specific bacteriophage specific for said Edwardsiella infecting an intestinal tract, wherein said at least one said lytic enzyme is specific for and has the ability to digest a cell wall of one of said Edwardsiella and is coded for by the same said bacteriophage capable of infecting said bacteria being digested; and
b) a pharmaceutically acceptable carrier for delivering said at least one lytic enzyme to the intestinal tract.
A composition for treating exposure to Edwardsiella, comprising:
a) an effective amount of at least one lytic enzyme genetically coded for by a specific bacteriophage specific for said Edwardsiella infecting an intestinal tract, wherein said at least one said lytic enzyme is specific for and has the ability to digest a cell wall of one of said Edwardsiella and is coded for by the same said bacteriophage capable of infecting said Edwardsiella being digested; and
b) a pharmaceutically acceptable carrier for delivering said at least one lytic enzyme to the intestinal tract.

83) A method of treating exposure to E. tarda causing gastroenteritis, comprising:
administering an effective amount of a therapeutic agent to an intestinal tract, said therapeutic agent comprising:
a) an effective amount of at least one lytic enzyme genetically coded for by a specific bacteriophage specific for said E. tarda infecting an intestinal tract, wherein said at least one said lytic enzyme is specific for and has the ability to digest a cell wall of one of said E. tarda and is coded for by the same said bacteriophage capable of infecting said bacteria being, digested; and
b) a pharmaceutically acceptable carrier for delivering said at least one lytic enzyme to the intestinal tract.
A composition for treating exposure to E. tarda, comprising:
a) an effective amount of at least one lytic enzyme genetically coded for by a specific bacteriophage specific for said E. tarda infecting an intestinal tract, wherein said at least one said lytic enzyme is specific for and has the ability to digest a cell wall of one of said E. tarda and is coded for by the same said bacteriophage capable of infecting said E. tarda being digested; and
b) a pharmaceutically acceptable carrier for delivering said at least one lytic enzyme to the intestinal tract.

84) A method of treating exposure to E. tarda causing gastroenteritis, comprising:
administering an effective amount of a therapeutic agent to an intestinal tract, said therapeutic agent comprising:
a) an effective amount of at least one lytic enzyme genetically coded for by a specific bacteriophage specific for said E. tarda infecting an intestinal tract, wherein said at least one said lytic enzyme is specific for and has the ability to digest a cell wall of one of said E. tarda and is coded for by the same said bacteriophage capable of infecting said bacteria being digested; and
b) a pharmaceutically acceptable carrier for delivering said at least one lytic enzyme to the intestinal tract.
A composition for treating exposure to Tropheryma whippelii, comprising:
a) an effective amount of at least one lytic enzyme genetically coded for by a specific bacteriophage specific for said Tropheryma whippelii infecting an intestinal tract, wherein said at least one said lytic enzyme is specific for and has the ability to digest a cell wall of one of said Tropheryma whippelii and is coded for by the same said bacteriophage capable of infecting said being digested; and
b) a pharmaceutically acceptable carrier for delivering said at least one lytic enzyme to the intestinal tract.

85) A method of treating exposure to Mycobacterium paratuberculosis causing gastroenteritis, comprising:
administering an effective amount of a therapeutic agent to an intestinal tract, said therapeutic agent comprising:
a) an effective amount of at least one lytic enzyme genetically coded for by a specific bacteriophage specific for said Mycobacterium paratuberculosis infecting an intestinal tract, wherein said at least one said lytic enzyme is specific for and has the ability to digest a cell wall of one of said Mycobacterium paratuberculosis and is coded for by the same said bacteriophage capable of infecting said bacteria being digested; and
b) a pharmaceutically acceptable carrier for delivering said at least one lytic enzyme to the intestinal tract.
A composition for treating exposure to Helicobacter comprising:
a) an effective amount of at least one lytic enzyme genetically coded for by a specific bacteriophage specific for said Helicobacter infecting an intestinal tract, wherein said at least one said lytic enzyme is specific for and has the ability to digest a cell wall of one of said Helicobacter and is coded for by the same said bacteriophage capable of infecting said being digested; and
b) a pharmaceutically acceptable carrier for delivering said at: least one lytic enzyme to the intestinal tract.

86) A method of treating exposure to Helicobacter causing gastroenteritis, comprising:
administering an effective amount of a therapeutic agent to an intestinal tract, said therapeutic agent comprising:
a) an effective amount of at least one lytic enzyme genetically coded for by a specific bacteriophage specific for said Helicobacter, infecting an intestinal tract, wherein said at least one said lytic enzyme is specific for and has the ability to digest a cell wall of one of said Helicobacter and is coded for by the same said bacteriophage capable of infecting said bacteria being digested; and
b) a pharmaceutically acceptable carrier for delivering said at least one lytic enzyme to the intestinal tract.

107. A canned food product, said canned food product comprising:
a) a food product;
b) a can in which said food product is packaged; and
c) an effective amount of at least one lytic enzyme genetically coded for by a specific bacteriophage specific for Clostridium bolulinum, wherein said at least one said lytic enzyme is specific for and has the ability to digest a cell wall of one of said Clostridium bolulinum and is coded for by the same said bacteriophage capable of infecting said Clostridium bolulinum being digested, wherein said at least one lytic enzyme is enclosed in said can in which said food product is packaged.
A composition for treating exposure to Salmonella typhisuis comprising:
a) an effective amount of at least one lytic enzyme genetically coded for by a specific bacteriophage specific for said Samonella typhisuis infecting salivary glands, wherein said at least one said lytic enzyme is specific for and has die ability to digest a cell wall of one of said Salmonella typhisuis and is coded for by the same said bacteriophage capable of infecting said being digested; and
b) a pharmaceutically acceptable carrier for delivering said at least one lytic enzyme to the salivary glands.

109) A method of treating Salmonella typhisuis causing inflammation of the salivary glands in pigs, said method comprising comprising:
administering an effective amount of a therapeutic agent to an intestinal tract, said therapeutic agent comprising:
a) an effective amount of at least one lytic enzyme genetically coded for by a specific bacteriophage specific for said Salmonella typhisuis infecting salivary glands, wherein said at least one said lytic enzyme is specific for and has the ability to digest a cell wall of said Salmonella typhisuis and is coded for by the same said bacteriophage capable of infecting said bacteria being digested; and
b) a pharmaceutically acceptable carrier for delivering said at least one lytic enzyme to the salivary glands.
A composition for treating exposure to Streptococcus bovis aid Lactobacillus spp. causing the production of lactic acid, which damages the ruminal mucosa in cattle, said method comprising:
a) an effective amount of at least one lytic enzyme genetically coded for by a specific bacteriophage specific for said Streptococcus bovis and said Lactobacillus ssp. infecting said ruminal mucosa, wherein said at least one said lytic enzyme is specific for and has the ability to digest a cell wall of one of said bacteria selected from the group consisting of Streptococcus bovis and Lactobacillus spp. and is coded for by the same said bacteriophage capable of infecting said bacteria being digested; and
b) a pharmaceutically acceptable carrier for delivering said at least one lytic enzyme to the ruminal musoca.
A method of treating Streptococcus bovis and Lactobacillus spp. resulting in the production of lactic
acid damaging the ruminal mucosa in cattle, said method comprising:
administering an effective amount of a therapeutic agent to the ruminal mucosa, said therapeutic agent comprising:
a) an effective amount of at least one lytic enzyme genetically coded for by a specific bacteriophage specific for said Streptococcus bovis and Lactobacillus spp. causing damage to said ruminal mucosa, wherein said at least one said lytic enzyme is specific for and has the ability to digest a cell wall of one of said bacteria selected from the group consisting of Streptococcus bovis and Lactobacillus spp. and is coded for by the same said bacteriophage capable of infecting said bacteria being digested; and
b) a pharmaceutically acceptable carrier for delivering said at least one lytic enzyme to the stomach of said cattle.
A composition for treating liver damage caused by Actinomyces pyogenes and Fusobacterium necrophorum, said composition comprising:
a) an effective amount of at least one lytic enzyme genetically coded for by a specific bacteriophage specific for said Actinomyces pyogenes and Fusobacterium necrophorum infecting said liver, wherein said at least one said lytic enzyme is specific for and has the ability to digest a cell wall of one of said bacteria selected from the group consisting of Actinomtyces pyogenes and Fusobacteriuin necrophorum and is coded for by the same said bacteriophage capable of infecting said bacteria being digested; and
b) a pharmaceutically acceptable carrier for delivering said at least one lytic enzyme to the liver.
A method of treating Actinomyces pyogenes and Fusobacterium necrophorum infections of the liver.
administering an effective amount of a therapeutic agent to an infected liver, said therapeutic agent comprising:
a) an effective amount of at least one lytic enzyme genetically coded for by a specific bacteriophage specific for said Actinomyces pyogenes and Fusobacteriuin necrophorum infecting said liver, wherein said at least one said lytic enzyme is specific for and has the ability to digest a cell wall of one of said bacteria selected from the group consisting of Actinomyces pyogenes and Fusobacterium necrophorum and is coded for by the same said bacteriophage capable of infecting said bacteria being digested; and
b) a pharmaceutically acceptable carrier for delivering said at least one lytic enzyme to the liver.

87) A composition for treating C. perfringens causing acute gastric dilation in horses and monkeys, comprising:
a) an effective amount of at least one lytic enzyme genetically coded for by a specific bacteriophage specific for said C. perfringens infecting the stomachs of horses and monkeys, wherein said at least one said lytic enzyme is specific for and has the ability to digest a cell wall of said C. peyfringens and is coded for by the same said bacteriophage capable of infecting said C. perfringens being digested; and
b) a pharmaceutically acceptable carrier for delivering said at least one lytic enzyme to the site of the acute gastric dilation.

88) A method of treating acute gastric dilation in horses and monkeys caused by C. pefringens,said method comprising:
administering an effective amount of a therapeutic agent to an intestinal tract, said therapeutic agent comprising:
a) an effective amount of at least one lytic enzyme genetically coded for by a specific bacteriophage specific for said C. perfringens, wherein said at least one said lytic enzyme is specific for and has the ability to digest a cell wall of said C. perfringens, for by the same said bacteriophage capable of infecting said bacteria being digested; and
b) a pharmaceutically acceptable carrier for delivering said at least one lytic enzyme to the intestinal tract.

89) A composition for treating C. perfringens type C causing infections of the bacteral tract of calves, foals, and piglets during the first few days of life, said composition comprising:
a) an effective amount of at least one lytic enzyme genetically coded for by a specific bacteriophage specific for said C. perfringens type C infecting the stomachs of horses and monkeys, wherein said at least one said lytic enzyme is specific for and has the ability to digest a cell wall of said C. perfringens type C and is coded for by the same said bacteriophage capable of infecting said C. perfringens type C being digested; and
b) a pharmaceutically acceptable carrier for delivering said at least one lytic enzyme to the site of the acute gastric dilation.

117) A method of treating intestinal disorders in calves, foals, and piglets in the first few days of life caused by C. perfringens type C, said method comprising:
administering an effective amount of a therapeutic agent to an intestinal tract, said therapeutic agent comprising:
a) an effective amount of at least one lytic enzyme genetically coded for by a specific bacteriophage specific for said C. perfringens type C, wherein said at least one said lytic enzyme is specific for and has the ability to digest a cell wall of said C. perfringens type C, for by the same said bacteriophage capable of infecting said bacteria being digested; and
b) a pharmaceutically acceptable carrier for delivering said at least one lytic enzyme to the intestinal tract.

118) A composition for treating C. perfringens, type D affecting the intestinal tract of fattening sheep, goats, and calves, comprising:
a) an effective amount of at least one lytic enzyme genetically coded for by a specific bacteriophage specific for said C. perfringens type D infecting the intestinal tract of fattening sheep, goats, and calves, wherein said at least one said lytic enzyme is specific for and has the ability to digest a cell wall of said C. perfringens type D and is coded for by the same said bacteriophage capable of infecting said C. perfringens type D being digested; and
b) a pharmaceutically acceptable carrier for delivering said at least one lytic enzyme to the site of the acute gastric dilation.

119) A method of treating intestinal infections in fattening sheeps, goats, and calves caused by C. perfringens type D, said method comprising:
administering an effective amount of a therapeutic agent to an intestinal tract, said therapeutic agent comprising:
a) an effective amount of at least one lytic enzyme genetically coded for by a specific bacteriophage specific for said C. perfringens type D, wherein said at least one said lytic enzyme is specific for and has the ability to digest a cell wall of said C. perfringens type D, for by the same said bacteriophage capable of infecting said bacteria being digested; and
b) a pharmaceutically acceptable carrier for delivering said at least one lytic enzyme to the intestinal tract.

120) A composition for treating a C. piliformis infection of mammalian species, said composition comprising:
a) an effective amount of at least one lytic enzyme genetically coded for by a specific bacteriophage specific for said C. piliformis infecting intestinal tracts of mammalian species, wherein said at least one said lytic enzyme is specific for and has the ability to digest a cell wall of said C. piliformis and is coded for by the same said bacteriophage capable of infecting said C. piliformis being digested; and
b) a pharmaceutically acceptable carrier for delivering said at least one lytic enzyme to the site of the bacterial infection.

121) A method for treating a C. piliformis infection of mammalian species, said method comprising:
administering an effective amount of a therapeutic agent to the site of the infection, said therapeutic agent comprising:
a) an effective amount of at least one lytic enzyme genetically coded for by a specific bacteriophage specific for said, wherein said at least one said lytic enzyme is specific for and has the ability to digest a cell wall of said C. piliformis, for by the same said bacteriophage capable of infecting said bacteria being digested; and
b) a pharmaceutically acceptable carrier for delivering said at least one lytic enzyme to the site of the infection.

122) A method of treating acute phlegomonous gastritis in dogs, said method comprising: administering an effective amount of a therapeutic agent to a gastric wall of said dogs, said therapeutic agent comprising:
a) an effective amount of at least one lytic enzyme genetically coded for by a specific bacteriophage specific for a bacteria infecting said cattle, wherein the bacteria to be treated is selected from the group consisting of Streptococci, staphylococci, E. coli, Proteus vulgaris, Clostridia perfringens, and combinations thereof, wherein said at least one said lytic enzyme is specific for and has the ability to digest a cell wall of one of said bacteria and is coded for by the same said bacteriophage capable of infecting said bacteria being digested; and b) a pharmaceutically acceptable carrier for delivering said at least one lytic enzyme to the gastric wall.

123) A method of treating hemorrhagic abomasitis in sheep and cattle caused by Clostridia septicum, said method comprising:
administering an effective amount of a therapeutic agent to a mouth, tongue, throat or lungs of said cattle, said therapeutic agent comprising:
a) an effective amount of at least one lytic enzyme genetically coded for by a specific bacteriophage specific for a bacteria infecting said cattle, wherein the bacteria to be treated is Clostridium septicum, said at least one said lytic enzyme is specific for and has the ability to digest a cell wall of said bacteria; and b) a pharmaceutically acceptable carrier for delivering said at least one lytic enzyme to the gastric wall.

124) A composition for treating a Clostridia septicum infection of sheep and cattle, said composition comprising:
a) an effective amount of at least one lytic enzyme genetically coded for by a specific bacteriophage specific for said Clostridia septicum infecting said sheep and said cattle, wherein said at least one said lytic enzyme is specific for and has the ability to digest a cell wall of said Clostridium septicum and is coded for by the same said bacteriophage capable of infecting said Clostridium septicum being digested; and
b) a pharmaceutically acceptable carrier for delivering said at least one lytic enzyme to the site of the bacterial infection.

125) A food product for effectively treating or preventing treating a Clostridium septicum infection of sheep and cattle, said composition comprising:
a) an effective amount of at least one lytic enzyme genetically coded for by a specific bacteriophage specific for said Clostridium septicum infecting intestinal tracts of said sheep and cattle, wherein said at least one said lytic enzyme is specific for and has the ability to digest a cell wall of said Clostridium septicum and is coded for by the same said bacteriophage capable of infecting said Clostridium septicum being digested; and
b) a food carrier for delivering said at least one lytic enzyme.

126) A method for treating a Clostridium septicum infection, said method comprising:
administering an effective amount of a therapeutic agent to the site of the infection, said therapeutic agent comprising:
a) an effective amount of at least one lytic enzyme genetically coded for by a specific bacteriophage specific for said, wherein said at least one said lytic enzyme is specific for and has the ability to digest a cell wall of said Clostridium septicum, for by the same said bacteriophage capable of infecting said bacteria being digested; and
b) a pharmaceutically acceptable carrier for delivering said at least one lytic enzyme to the site of the infection.

127) A method of treating intestinal tuberculosis in calves, cattle, pigs and dogs, said method comprising:
administering an effective amount of a therapeutic agent to the site of the infection in an infected animal, said therapeutic agent comprising:
a) an effective amount of at least one lytic enzyme genetically coded for by a specific bacteriophage specific for a bacteria infecting said cattle, wherein the bacteria to be treated is selected from the group consisting of Mycobacterium tuberculosis, Mycobacterium bovis, and combinations thereof, wherein said at least one said lytic enzyme is specific for and has the to ability to digest a cell wall of one of said bacteria and is coded for by the same said bacteriophage capable of infecting said bacteria being digested; and
b) a pharmaceutically acceptable carrier for delivering said at least one lytic enzyme to the intestinal tract.
